SEQUENCE LISTING (54 sequences)

SEQ ID NO: 1 - DNA, 2077 bp, Homo sapiens
agcgcagcgtgcgggtggcctggatcccgcgcagtggcccggcgatgtcgctcgtgctgctaagcctggccgcgctgtgcaggagcgccgtaccccgagagccgaccgttcaatgtggctctgaaactgggccatctccagagtggatgctacaacatgatctaatccccggagacttgagggacctccgagtagaacctgttacaactagtgttgcaacaggggactattcaattttgatgaatgtaagctgggtactccgggcagatgccagcatccgcttgttgaaggccaccaagatttgtgtgacgggcaaaagcaacttccagtcctacagctgtgtgaggtgcaattacacagaggccttccagactcagaccagaccctctggtggtaaatggacattttcctacatcggcttccctgtagagctgaacacagtctatttcattggggcccataatattcctaatgcaaatatgaatgaagatggcccttccatgtctgtgaatttcacctcaccaggctgcctagaccacataatgaaatataaaaaaaagtgtgtcaaggccggaagcctgtgggatccgaacatcactgcttgtaagaagaatgaggagacagtagaagtgaacttcacaaccactcccctgggaaacagatacatggctcttatccaacacagcactatcatcgggttttctcaggtgtttgagccacaccagaagaaacaaacgcgagcttcagtggtgattccagtgactggggatagtgaaggtgctacggtgcagctgactccatattttcctacttgtggcagcgactgcatccgacataaaggaacagttgtgctctgcccacaaacaggcgtccctttccctctggataacaacaaaagcaagccgggaggctggctgcctctcctcctgctgtctctgctggtggccacatgggtgctggtggcagggatctatctaatgtggaggcacgaaaggatcaagaagacttccttttctaccaccacactactgccccccattaaggttcttgtggtttacccatctgaaatatgtttccatcacacaatttgttacttcactgaatttcttcaaaaccattgcagaagtgaggtcatccttgaaaagtggcagaaaaagaaaatagcagagatgggtccagtgcagtggcttgccactcaaaagaaggcagcagacaaagtcgtcttccttctttccaatgacgtcaacagtgtgtgcgatggtacctgtggcaagagcgagggcagtcccagtgagaactctcaagacctcttcccccttgcctttaaccttttctgcagtgatctaagaagccagattcatctgcacaaatacgtggtggtctactttagagagattgatacaaaagacgattacaatgctctcagtgtctgccccaagtaccacctcatgaaggatgccactgctttctgtgcagaacttctccatgtcaagcagcaggtgtcagcaggaaaaagatcacaagcctgccacgatggctgctgctccttgtagcccacccatgagaagcaagagaccttaaaggcttcctatcccaccaattacagggaaaaaacgtgtgatgatcctgaagcttactatgcagcctacaaacagccttagtaattaaaacattttataccaataaaattttcaaatattgctaactaatgtagcattaactaacgattggaaactacatttacaacttcaaagctgttttatacatagaaatcaattacagttttaattgaaaactataaccattttgataatgcaacaataaagcatcttcagccaaacatctagtcttccatagaccatgcattgcagtgtacccagaactgtttagctaatattctatgtttaattaatgaatactaactctaagaacccctcactgattcactcaatagcatcttaagtgaaaaaccttctattacatgcaaaaaatcattgtttttaagataacaaaagtagggaataaacaagctgaacccacttttaaaaaaaaaaaaaaaaaaaaaaaaaaaaaaaaa

SEQ ID NO: 2 - DNA, 3105 bp, Homo sapiens
agcgcagcgtgcgggtggcctggatcccgcgcagtggcccggcgatgtcgctcgtgctgctaagcctggccgcgctgtgcaggagcgccgtaccccgagagccgaccgttcaatgtggctctgaaactgggccatctccagagtggatgctacaacatgatctaatccccggagacttgagggacctccgagtagaacctgttacaactagtgttgcaacaggggactattcaattttgatgaatgtaagctgggtactccgggcagatgccagcatccgcttgttgaaggccaccaagatttgtgtgacgggcaaaagcaacttccagtcctacagctgtgtgaggtgcaattacacagaggccttccagactcagaccagaccctctggtggtaaatggacattttcctacatcggcttccctgtagagctgaacacagtctatttcattggggcccataatattcctaatgcaaatatgaatgaagatggcccttccatgtctgtgaatttcacctcaccaggctgcctagaccacataatgaaatataaaaaaaagtgtgtcaaggccggaagcctgtgggatccgaacatcactgcttgtaagaagaatgaggagacagtagaagtgaacttcacaaccactcccctgggaaacagatacatggctcttatccaacacagcactatcatcgggttttctcaggtgtttgagccacaccagaagaaacaaacgcgagcttcagtggtgattccagtgactggggatagtgaaggtgctacggtgcaggtaaagttcagtgagctgctctggggagggaagggacatagaagactgttccatcattcattgcttttaaggatgagttctctcttgtcaaatgcacttctgccagcagacaccagttaagtggcgttcatgggggctctttcgctgcagcctccaccgtgctgaggtcaggaggccgacgtggcagttgtggtcccttttgcttgtattaatggctgctgaccttccaaagcactttttattttcattttctgtcacagacactcagggatagcagtaccattttacttccgcaagcctttaactgcaagatgaagctgcaaagggtttgaaatgggaaggtttgagttccaggcagcgtatgaactctggagaggggctgccagtcctctctgggccgcagcggacccagctggaacacaggaagttggagcagtaggtgctccttcacctctcagtatgtctctttcaactctagtttttgaggtggggacacaggaggtccagtgggacacagccactccccaaagagtaaggagcttccatgcttcattccctggcataaaaagtgctcaaacacaccagagggggcaggcaccagccagggtatgatggctactacccttttctggagaaccatagacttcccttactacagggacttgcatgtcctaaagcactggctgaaggaagccaagaggatcactgctgctccttttttctagaggaaatgtttgtctacgtggtaagatatgacctagcccttttaggtaagcgaactggtatgttagtaacgtgtacaaagtttaggttcagaccccgggagtcttgggcacgtgggtctcgggtcactggttttgactttagggctttgttacagatgtgtgaccaaggggaaaatgtgcatgacaacactagaggtatgggcgaagccagaaagaagggaagttttggctgaagtaggagtcttggtgagattttgctctgatgcatggtgtgaactttctgagcctcttgtttttcctcagctgactccatattttcctacttgtggcagcgactgcatccgacataaaggaacagttgtgctctgcccacaaacaggcgtccctttccctctggataacaacaaaagcaagccgggaggctggctgcctctcctcctgctgtctctgctggtggccacatgggtgctggtggcagggatctatctaatgtggaggcacgaaaggatcaagaagacttccttttctaccaccacactactgccccccattaaggttcttgtggtttacccatctgaaatatgtttccatcacacaatttgttacttcactgaatttcttcaaaaccattgcagaagtgaggtcatccttgaaaagtggcagaaaaagaaaatagcagagatgggtccagtgcagtggcttgccactcaaaagaaggcagcagacaaagtcgtcttccttctttccaatgacgtcaacagtgtgtgcgatggtacctgtggcaagagcgagggcagtcccagtgagaactctcaagacctcttcccccttgcctttaaccttttctgcagtgatctaagaagccagattcatctgcacaaatacgtggtggtctactttagagagattgatacaaaagacgattacaatgctctcagtgtctgccccaagtaccacctcatgaaggatgccactgctttctgtgcagaacttctccatgtcaagcagcaggtgtcagcaggaaaaagatcacaagcctgccacgatggctgctgctccttgtagcccacccatgagaagcaagagaccttaaaggcttcctatcccaccaattacagggaaaaaacgtgtgatgatcctgaagcttactatgcagcctacaaacagccttagtaattaaaacattttataccaataaaattttcaaatattgctaactaatgtagcattaactaacgattggaaactacatttacaacttcaaagctgttttatacatagaaatcaattacagttttaattgaaaactataaccattttgataatgcaacaataaagcatcttcagccaaacatctagtcttccatagaccatgcattgcagtgtacccagaactgtttagctaatattctatgtttaattaatgaatactaactctaagaacccctcactgattcactcaatagcatcttaagtgaaaaaccttctattacatgcaaaaaatcattgtttttaagataacaaaagtagggaataaacaagctgaacccacttttaaaaaaaaaaaaaaaaaaaaaaaaaaaaaaaaa

SEQ ID NO: 3 - DNA, 2856 bp, Homo sapiens
  misc_feature (1325)..(1325): a or g or c or t/u
cggcgatgtcgctcgtgctgataagcctggccgcgctgtgcaggagcgccgtaccccgagagccgaccgttcaatgtggctctgaaactgggccatctccagagtggatgctacaacatgatctaatccccggagacttgagggacctccgagtagaacctgttacaactagtgttgcaacaggggactattcaattttgatgaatgtaagctgggtactccgggcagatgccagcatccgcttgttgaaggccaccaagatttgtgtgacgggcaaaagcaacttccagtcctacagctgtgtgaggtgcaattacacagaggccttccagactcagaccagaccctctggtggtaaatggacattttcctatatcggcttccctgtagagctgaacacagtctatttcattggggcccataatattcctaatgcaaatatgaatgaagatggcccttccatgtctgtgaatttcacctcaccaggctgcctagaccacataatgaaatataaaaaaaagtgtgtcaaggccggaagcctgtgggatccgaacatcactgcttgtaagaagaatgaggagacagtagaagtgaacttcacaaccactcccctgggaaacagatacatggctcttatccaacacagcactatcatcgggttttctcaggtgtttgagccacaccagaagaaacaaacgcgagcttcagtggtgattccagtgactggggatagtgaaggtgctacggtgcaggtaaagttcagtgagctgctctggggagggaagggacatagaagactgttccatcattcattgcttttaaggatgagttctctcttgtcaaatgcacttctgccagcagacaccagttaagtggcgttcatgggggttctttcgctgcagcctccaccgtgctgaggtcaggaggccgacgtggcagttgtggtcccttttgcttgtattaatggctgctgaccttccaaagcactttttattttcattttctgtcacagacactcagggatagcagtaccattttacttccgcaagcctttaactgcaagatgaagctgcaaagggtttgaaatgggaaggtttgagttccaggcagcgtatgaactctggagaggggctgccagtcctctctgggccgcagcggacccagctggaacacaggaagttggagcagtaggtgctccttcacctctcagtatgtctctttcaactctagtttttgaagtggggacacaggaagtccagtggggacacagccactccccaaagaataaggaacttccatgcttcattccctggcataaaaagtgntcaaacacaccagagggggcaggcaccagccagggtatgatgggtactacccttttctggagaaccatagacttcccttactacagggacttgcatgtcctaaagcactggctgaaggaagccaagaggatcactgctgctccttttttgtagaggaaatgtttgtgtacgtggtaagatatgacctagcccttttaggtaagcgaactggtatgttagtaacgtgtacaaagtttaggttcagaccccgggagtcttgggcatgtgggtctcgggtcactggttttgactttagggctttgttacagatgtgtgaccaaggggaaaatgtgcatgacaacactagaggtaggggcgaagccagaaagaagggaagttttggctgaagtaggagtcttggtgagattttgctgtgatgcatggtgtgaactttctgagcctcttgtttttcctcagctgactccatattttcctacttgtggcagcgactgcatccgacataaaggaacagttgtgctctgcccacaaacaggcgtccctttccctctggataacaacaaaagcaagccgggaggctggctgcctctcctcctgctgtctctgctggtggccacatgggtgctggtggcagggatctatctaatgtggaggcacgaaaggatcaagaagacttccttttctaccaccacactactgccccccattaaggttcttgtggtttacccatctgaaatatgtttccatcacacaatttgttacttcactgaatttcttcaaaaccattgcagaagtgaggtcatccttgaaaagtggcagaaaaagaaaatagcagagatgggtccagtgcagtggcttgccactcaaaagaaggcagcagacaaagtcgtcttccttctttccaatgacgtcaacagtgtgtgcgatggtacctgtggcaagagcgagggcagtcccagtgagaactctcaagacctcttcccccttgcctttaaccttttctgcagtgatctaagaagccagattcatctgcacaaatacgtggtggtctactttagagagattgatacaaaagacgattacaatgctctcagtgtctgccccaagtaccacttcatgaaggatgccactgctttctgtgcagaacttctccatgtcaagcagcaggtgtcagcaggaaaaagatcacaagcctgccacgatggctgctgctccttgtagcccacccatgagaagcaagagaccttaaaggcttcctatcccaccaattacagggaaaaaacgtgtgatgatcctgaagcttactatgcagcctacaaacagccttagtaattaaaacattttataccaataaaattttcaaatattactaactaatgtagcattaactaacgattggaaactacatttacaacttcaaagctgttttatacatagaaatcaattacagctttaattgaaaactgtaaccattttgataatgcaacaataaagcatcttccaaaaaaaaaaaaaaaaaaaaaaaaaaaaaaaa

SEQ ID NO: 4 - DNA, 2162 bp, Homo sapiens
agcgggccgcggccacccgctcctcccgctccggtcccgactgtcgggctctcggccgagtcgccccggacaatcacaaagagtgtgtaggccagccccggtcacagagtgcaccgtatcctgtcacttctggatgtgagggagaagtgagtcatctcattcccctccgtggatcagaggacttggactagatagaagcatgtggtgtctcctacgaggcctgggccggcctggagccctggcacggggagccctggggcagcagcaatccctgggtgcccgggccctggccagcgcaggctctgagagccgggacgagtacagctatgtggtggtgggcgcgggctcggcgggctgcgtgctggctgggaggctcacggaggaccccgccgagcgcgtgctgctgctggaggccgggcccaaggacgtgcgcgcggggagcaagcggctctcgtggaagatccacatgcccgcggccctggtggccaacctgtgcgacgacaggtacaactggtgctaccacacagaggtgcagcggggcctggacggccgcgtgctgtactggccacgcggccgcgtctggggtggctcctcatccctcaatgccatggtctacgtccgtgggcacgccgaggactacgagcgctggcagcgccagggcgcccgcggctgggactacgcgcactgcctgccctacttccgcaaggcgcagggccacgagctgggcgccagccggtaccggggcgccgatggcccgctgcgggtgtcccggggcaagaccaaccacccgctgcactgcgcattcctggaggccacgcagcaggccggctacccgctcaccgaggacatgaatggcttccagcaggagggcttcggctggatggacatgaccatccatgaaggcaaacggtggagcgcagcctgtgcctacctgcacccagcactgagccgcaccaacctcaaggccgaggccgagacgcttgtgagcagggtgctatttgagggcacccgtgcagtgggcgtggagtatgtcaagaatggccagagccacagggcttatgccagcaaggaggtgattctgagtggaggtgccatcaactctccacagctgctcatgctctctggcatcgggaatgctgatgacctcaagaaactgggcatccctgtggtgtgccacctacctggggttggccagaacctgcaagaccacctggagatctacattcagcaggcatgcacccgccctatcaccctccattcagcacagaagcccctgcggaaggtctgcattggtctggagtggctctggaaattcacaggggagggagccactgcccatctggaaacaggtgggttcatccgcagccagcctggggtcccccacccggacatccagttccatttcctgccatcccaagtgattgaccacgggcgggtccccacccagcaggaggcttaccaggtacatgtggggcccatgcggggcacgagtgtgggctggctcaaactgagaagtgccaatccccaagaccaccctgtgatccagcccaactacttgtcaacagaaactgatattgaggatttccgtctgtgtgtgaagctcaccagagaaatttttgcacaggaagccctggctccgttccgagggaaagagctccagccaggaagccacattcagtcagataaagagatagatgcctttgtgcgggcaaaagccgacagcgcctaccacccctcgtgcacctgtaagatgggccagccctccgatcccactgccgtggtggatccgcagacaagggtcctcggggtggaaaacctcagggtcgtcgatgcctccatcatgcctagcatggtcagcggcaacctgaacgcccccacaatcatgatcgcagagaaggcagctgacattatcaaggggcagcctgcactctgggacaaagatgtccctgtctacaagcccaggacgctggccacccagcgctaagacagttgctgctggaggatgaccagggaagccccctgataagccaagagggccagcacagcccttgctcccaggctcctgcctgaaactatctagcacactaggacccaggtggtaccctactcagtggctgagaattggataaagtcttgggaaatgagacaaaaaaaaaaaaaaa

SEQ ID NO: 5 - DNA, 1182 bp, Homo sapiens
gtcctgagcagccaacacaccagcccagacagctgcaagtcaccatggacgctgaaggcctggcgctgctgctgccgcccgtcaccctggcagccctggtggacagctggctccgagaggactgcccagggctcaactacgcagccttggtcagcggggcaggcccctcgcaggcggcgctgtgggccaaatcccctggggtactggcagggcagcctttcttcgatgccatatttacccaactcaactgccaagtctcctggttcctccccgagggatcgaagctggtgccggtggccagagtggccgaggtccggggccctgcccactgcctgctgctgggggaacgggtggccctcaacacgctggcccgctgcagtggcattgccagtgctgccgccgctgcagtggaggccgccaggggggccggctggactgggcacgtggcaggcacgaggaagaccacgccaggcttccggctggtggagaagtatgggctcctggtgggcggggccgcctcgcaccgctacgacctgggagggctggtgatgttgaaggataaccatgtggtgccccccggtggcgtggagaaggcggtgcgggcggccagacaggcggctgacttcgctctgaaggtggaagtggaatgcagcagcctgcaggaggtcgtccaggcagctgaggctggcgccgaccttgtcctgctggacaacttcaagccagaggagctgcaccccacggccaccgcgctgaaggcccagttcccgagtgtggctgtggaagccagtgggggcatcaccctggacaacctcccccagttctgcgggccgcacatagacgtcatctccatggggatgctgacccaggcggtcccagcccttgatttctccctcaagctgtttgccaaagaggtggctccagtgcccaaaatccactagtcctaaaccggaagaggatgacaccggccatgggttaacgtggctcctcaggaccctctgggtcacacatctttagggtcagtgaacaatggggcacatttggcactagcttgagcccaactctggctctgccacctgctgctcctgtgacctgtcagggctgacttcacctctgctcatctcagtttcctaatctgtaaaatgggtctaataaaggatcaaccaaaaaaaaaaaaaaaaaaaa

SEQ ID NO: 6 - DNA, 1270 bp, Homo sapiens
cgaatgcaggcgacttgcgagctgggagcgatttaaaacgctttggattcccccggcctgggtggggagagcgagctgggtgccccctagattccccgcccccgcacctcatgagccgaccctcggctccatggagcccggcaattatgccaccttggatggagccaaggatatcgaaggcttgctgggagcgggaggggggcggaatctggtcgcccactcccctctgaccagccacccagcggcgcctacgctgatgcctgctgtcaactatgcccccttggatctgccaggctcggcggagccgccaaagcaatgccacccatgccctggggtgccccaggggacgtccccagctcccgtgccttatggttactttggaggcgggtactactcctgccgagtgtcccggagctcgctgaaaccctgtgcccaggcagccaccctggccgcgtaccccgcggagactcccacggccggggaagagtaccccagtcgccccactgagtttgccttctatccgggatatccgggaacctaccacgctatggccagttacctggacgtgtctgtggtgcagactctgggtgctcctggagaaccgcgacatgactccctgttgcctgtggacagttaccagtcttgggctctcgctggtggctggaacagccagatgtgttgccagggagaacagaacccaccaggtcccttttggaaggcagcatttgcagactccagcgggcagcaccctcctgacgcctgcgcctttcgtcgcggccgcaagaaacgcattccgtacagcaaggggcagttgcgggagctggagcgggagtatgcggctaacaagttcatcaccaaggacaagaggcgcaagatctcggcagccaccagcctctcggagcgccagattaccatctggtttcagaaccgccgggtcaaagagaagaaggttctcgccaaggtgaagaacagcgctaccccttaagagatctccttgcctgggtgggaggagcgaaagtgggggtgtcctggggagaccagaaacctgccaagcccaggctggggccaaggactctgctgagaggcccctagagacaacacccttcccaggccactggctgctggactgttcctcaggagcggcctgggtacccagtatgtgcagggagacggaaccccatgtgacaggcccactccaccagggttcccaaagaacctggcccagtcataatcattcatcctcacagtggcaataatcacgataaccagt

SEQ ID NO: 7 - DNA, 1356 bp, Homo sapiens
ggattcccccggcctgggtggggagagcgagctgggtgccccctagattccccgcccccgcacctcatgagccgaccctcggctccatggagcccggcaattatgccaccttggatggagccaaggatatcgaaggcttgctgggagcgggaggggggcggaatctggtcgcccactcccctctgaccagccacccagcggcgcctacgctgatgcctgctgtcaactatgcccccttggatctgccaggctcggcggagccgccaaagcaatgccacccatgccctggggtgccccaggggacgtccccagctcccgtgccttatggttactttggaggcgggtactactcctgccgagtgtcccggagctcgctgaaaccctgtgcccaggcagccaccctggccgcgtaccccgcggagactcccacggccggggaagagtaccccagccgccccactgagtttgccttctatccgggatatccgggaacctaccagcctatggccagttacctggacgtgtctgtggtgcagactctgggtgctcctggagaaccgcgacatgactccctgttgcctgtggacagttaccagtcttgggctctcgctggtggctggaacagccagatgtgttgccagggagaacagaacccaccaggtcccttttggaaggcagcatttgcagactccagcgggcagcaccctcctgacgcctgcgcctttcgtcgcggccgcaagaaacgcattccgtacagcaaggggcagttgcgggagctggagcgggagtatgcggctaacaagttcatcaccaaggacaagaggcgcaagatctcggcagccaccagcctctcggagcgccagattaccatctggtttcagaaccgccgggtcaaagagaagaaggttctcgccaaggtgaagaacagcgctaccccttaagagatctccttgcctgggtgggaggagcgaaagtgggggtgtcctggggagaccaggaacctgccaagcccaggctggggccaaggactctgctgagaggcccctagagacaacacccttcccaggccactggctgctggactgttcctcaggagcggcctgggtacccagtatgtgcagggagacggaaccccatgtgacagcccactccaccagggttcccaaagaacctggcccagtcataatcattcatcctgacagtggcaataatcacgataaccagtactagctgccatgatcgttagcctcatattttctatctagagctctgtagagcactttagaaaccgctttcatgaattgagctaattatgaataaatttggaaaaaaaaaaaaaaaaaaaaaaaaaaaaaa

SEQ ID NO: 8 - DNA, 60 bp, Homo sapiens
gctctcactggcaaatgacagctctgtgcaaggagcactcccaagtataaaaattattac

SEQ ID NO: 9 - DNA, 60 bp, Homo sapiens
tgcctaatttcactctcagagtgaggcaggtaactggggctccactgggtcactctgaga

SEQ ID NO: 10 - DNA, 60 bp, Homo sapiens
gatcgttagcctcatattttctatctagagctctgtagagcactttagaaaccgctttca

SEQ ID NO: 11 - DNA, 29 bp, Homo sapiens
tccaatcgttagttaatgctacattagtt

SEQ ID NO: 12 - DNA, 25 bp, Homo sapiens
caattcatgaaagcggtttctaaag

SEQ ID NO: 13 - DNA, 22 bp, Artificial Sequence
  Forward primer
tgaagtgtttttgcctggatca

SEQ ID NO: 14 - DNA, 27 bp, Artificial Sequence
  Reverse primer
caccactttgttatgaagaccttacaa

SEQ ID NO: 15 - DNA, 23 bp, Artificial Sequence
  Probe for detecting A1240933 expression
agtaagaatgtcttaagaagagg

SEQ ID NO: 16 - DNA, 24 bp, Artificial Sequence
  Forward primer used in PCR analysis of HoxB13
ttcatcctgacagtggcaataatc

SEQ ID NO: 17 - DNA, 30 bp, Artificial Sequence
  Reverse primer used in PCR analysis of HoxB13
ctagatagaaaatatgaggctaacgatcat

SEQ ID NO: 18 - DNA, 19 bp, Artificial Sequence
  Fluorogenic MGB probe used in PCR analysis of HoxB13
cgataaccagtactagctg

SEQ ID NO: 19 - DNA, 28 bp, Artificial Sequence
  Forward primer used in PCR analysis of IL17BR
gcattaactaacgattggaaactacatt

SEQ ID NO: 20 - DNA, 26 bp, Artificial Sequence
  Reverse primer used in PCR analysis of IL17BR
ggaagatgctttattgttgcattatc

SEQ ID NO: 21 - DNA, 20 bp, Artificial Sequence
  Fluorogenic MGB probe used in PCR analysis of IL17BR
acaacttcaaagctgtttta

SEQ ID NO: 22 - DNA, 24 bp, Artificial Sequence
  Forward CHDH primer
aaagtcttgggaaatgagacaagt

SEQ ID NO: 23 - DNA, 21 bp, Artificial Sequence
  Reverse primer 83R
agctgtcatttgccagtgaga

SEQ ID NO: 24 - DNA, 21 bp, Artificial Sequence
  Reverse primer 81R
ctgtcatttgccagtgagagc

SEQ ID NO: 25 - DNA, 611 bp, Homo sapiens
ggaaaaacatttcatcttaaaactttcctaagacaagggaaaacaaaaaaccatgctctacaacttcaaatttttcttacaaagaaaaatttaatattcgatgagaggttgaaccaggcttaaagcagacatactaggaaatggtgcagcctgtaagaatgccagtttgtaagtactgagcctgatgtgatgccacaagacccaacagagagagacacagagtccaggataatgttgacagtggtgtagccctttaggagaaatggcgctccctgcggctggtattaggttaccattggcaccgaaggaaccaggaggataagaatatccataatttcagagctgccctggcacagtacctgccctgtcggaggctctcactggcaaatgacagctctgtgcaaggagcactcccaagtataaaaattattacacagttttattctgaagaacattttgcattttaataaaaaaggatttatgtcaggaaagagtcatttacaaaccttgaagtgtttttgcctggatcagagtaagaatgtcttaagaagaggtttgtaaggtcttcataaccaaagtggtgtttgttatttacaaaaaaaaaaaaaaaa

SEQ ID NO: 26 - DNA, 236 bp, Homo sapiens
  misc_feature (42)..(42): a or g or c or t/u
gataatgttgacagtggtgtagccctttaggagaaatggcgngtcctgcggctggtattaggttaccattgagcacagaaggaaccaggaggataagaatatccataatttcagagctgccctggcacagtacctgcctgtcgaggctctcactggcaaatgacagctctgtgcaaggagcacatcccaagtataaaaattattacacagttttattctgaagaacattttgcatt

SEQ ID NO: 27 - DNA, 400 bp, Homo sapiens
tttttcttacaaagaaaaatttaatattcgatgagaggttgaaccaggcttaaagcagacatactaggaaatggtgcagcctgtaagaatgccagtttgtaagtactgactttggaaaagatcatcgcctctatcagacacttagggtcctggtctggcaattttggcctgatgtgatgccacaagacccaacagagagagacacagagtccaggataatgttgacagtggtgtagccctttaggagaaatggcgctccctgcggctggtattaggttaccattggcaccgaaggaaccaggaggataagaatatccataatttcagagctgccctggcacagtacctgccccgtcggaggctctcactggcaaatgacagctctgtgcaaggagcactc

SEQ ID NO: 28 - DNA, 434 bp, Homo sapiens
tttttcttacaaagaaaaatttaatattcgatgagaggttgaaccaggcttaaagcagacatactaggaaatggtgcagcctgtaagaatgccagtttgtaagtactgactttggaaaagatcatcgcctctatcagacacttagggtcctggtctggcaattttggcctgatgtgatgccacaagacccaacagagagagacacagagtccaggataatgttgacagtggtgtagccctttaggagaaatggcgctccctgcggctggtattaggttaccattggcaccgaagagaccaggaggataagaatatccataatttcagagctgccctggcacagtacctgccccgtcggaggctctcactggcaaatgacagctctgtgcaaggagcactcccaagtataaaaattattacacagttttattctg

SEQ ID NO: 29 - DNA, 412 bp, Homo sapiens
cttacaaagaaaaatttaatattcgatgagaggttgaaccaggcttaaagcagacatactaggaaatggtgcagcctgtaagaatgccagtttgtaagtactgactttggaaaagatcatcgcctctatcagacacttagggtcctggtctggcaattttggcctgatgtgatgccacaagacccaacagagagagacacagagtccaggataatgttgacagtggtgtagccctttaggagaaatggcgctccctgcggctggtattaggttaccattggcaccgaagagaccaggaggataagaatatccataatttcagagctgccctggcacagtacctgccccgtcggaggctctcactggcaaatgacagctctgtgcaaggagcactcccaagtataaaaattat

SEQ ID NO: 30 - DNA, 603 bp, Homo sapiens
  misc_feature (33)..(33): a or g or c or t/u
tttttcttacaaagaaaaatttaatattcgatngagaggttgaaccaggcttaaagcagacatactaggaaatggtgcagcctgtaagaatgccagtttgtaagtactgactttggaaaagatcatcgcctctatcagacacttagggtcctggtctggcaattttggcctgatgtgatgccacaagacccaacagagagagacacagagtccaggataatgttgacagtggtgtagccctttaggagaaatggcgctccctgcggctggtattaggttaccattggcaccgaaggaaccaggaggataagaatatccataatttcagagctgccctggcacagtacctgccccgtcggaggctctcactggcaaatgacagctctgtgcaaggagcactcccaagtataaaaattattacacagttttattctgaagaacattttgcattttaataaaaaaggatttatgtcaggaaagagtcatttacaaaccttgaagtgtttttgcctggatcagagtaagaatgtcttaagaagaggtttgtaaggtcttcataacanagtggtgtttgttatttacaaaaaaaaaaaaaaaaaaaat

SEQ ID NO: 31 - DNA, 410 bp, Homo sapiens
caagacccaacagagagagacacagagtccaggataatgttgacagtggtgtagccctttaggagaaatggcgctccctgcggctggtattaggttaccattggcaccgaaggaaccaggaggataagaatatccataatttcagagctgccctggcacagtacctgccccgtcggaggctctcactggcaaatgacagctctgtgcaaggagcactcccaagtataaaaattattacacagttttattctgaagaacattttgcattttaataaaaaaggatttatgtcaggaaagagtcatttacaaaccttgaagtgtttttgcctggatcagagtaagaatgtcttaagaagaggtttgtaaggtcttcataacaaagtggtgtttgttatttacaaaaaaaaaaa

SEQ ID NO: 32 - DNA, 487 bp, Homo sapiens
tgtaagtactgactttggaaaagatcatcgccttctatcagaccacttggggtcctggtctggccaattttggcctgatgtgatgccacaagacccaacagagagagacacagagtcccaggataatgttgacagtggtgtagccctttaggagaaatggcgctcctgcggctggtattaggttaccattggcaccgaaggaaccaggaggataagaatatccataatttcagagctgccctggcacagtacctgccccgtcggaggctctcactggcaaatgacagctctgtgcaaggagcactcccaagtataaaaattattacacagttttattctgaagaacattttgcattttaataaaaaaggatttatgtcaggaaagagtcatttacaaaccttgaagtgtttttgcctggatcagagtaagaatgtcttaagaagaggtttgtaaggtcttcataacaaagtggtgtttgttatttac

SEQ ID NO: 33 - DNA, 473 bp, Homo sapiens
aagatcatcgcctctatcagacacttagggtcctggtctggcaattttggcctgatgtgatgccacaagactcaacagagagagacacagagtccaggataatgttgacagtggtgtagccctttaggagaaatggcgctccctgcggctggtattaggttaccattggcaccgaaggaaccaggaggataagaatatccataatttcagagctgccctggcacagtacctgccctgtcggaggctctcactggcaaatgacagctctgtgcaaggagcactcccaagtataaaaattattacacagttttattctgaagaacattttgcattttaataaaaaaggatttatgtcaggaaagagtcatttacaaaccttgaagtgtttttgcctggatcagagtaagaatgtcttaagaagaggtttgtaaggtcttcataacaaagtggtgtttgttatttacaaaaaaaaa

SEQ ID NO: 34 - DNA, 491 bp, Homo sapiens
agtttgtaagtactgactttggaaaagatcatcgcctctatcagacacttagggtcctggtctggcaattttggcctgatgtgatgccacaagacccaacagagagagacacagagtccaggataatgttgacagtggtgtagccctttaggagaaatggcgctccctgcggctggtattaggttaccattggcaccgaaggaaccaggaggataagaatatccataatttcagagctgccctggcacagtacctgccccgtcggaggctctcactggcaaatgacagctctgtgcaaggagcactcccaagtataaaaattattacacagttttattctgaagaacattttgcattttaataaaaaaggatttatgtcaggaaagagtcatttacaaaccttgaagtgtttttgcctggatcagagtaagaatgtcttaagaagaggtttgtaaggtcttcataacaaagtggtgtttgttatttacaaa

SEQ ID NO: 35 - DNA, 483 bp, Homo sapiens
taagtactgactttggaaaagatcatcgcctctatcagacacttagggtcctggtctggcaattttggcctgatgtgatgccacaagacccaacagagagagacacagagtccaggataatgttgacagtggtgtagccctttaggagaaatggcgctcctgcggctggtattaggttaccattggcaccgaaggaaccaggaggataagaatatccataatttcagagctgccctggcacagtacctgccccttcggaggctctcactggcaaatgacagctctgtgcaaggagcactcccaagtataaaaattattacacagttttattctgaagaacattttgcattttaataaaaaaggatttatgtcaggaaagagtcatttacaaaccttgaagtgtttttgcctggatcagagtaagaatgtcttaagaagaggtttgtaaggtcttcataacaaagtggtgtttgttatttacaa

SEQ ID NO: 36 - DNA, 502 bp, Homo sapiens
ctgtaagaatgccagtttgtaagtactgactttggaaaagatcatcgcctctatcagacacttagggtcctggtctggcaattttggcctgatgtgatgccacaagacccaacagagagagacacagagtccaggataatgttgacagtggtgtagccctttaggagaaatggcgctccctgcggctggtattaggttaccattggcaccgaaggaaccaggaggataagaatatccataatttcagagctgccctggcacagtacctgccccgtcggaggctctcactggcaaatgacagctctgtgcaaggagcactcccaagtataaaaattattacacagttttattctgaagaacattttgcattttaataaaaaaggatttatgtcaggaaagagtcatttacaaaccttgaagtgtttttgcctggatcagagtaagaatgtcttaagaagaggtttgtaaggtcttcataacaaagtggtgtttgttatttaca

SEQ ID NO: 37 - DNA, 548 bp, Homo sapiens
ggttgaaccaggcttaaagcagacatactaggaaatggtgcagcctgtaagaatgccagtttgtaagtactgactttggaaaagatcatcgcctctatcagacacttagggtcctggtctggcaattttggcctgatgtgatgccacaagacccaacagagagagacacagagtccaggataatgttgacagtggtgtagccctttaggagaaatggcgctccctgcggctggtattaggttaccattggcaccgaaggaaccaggaggataagaatatccataatttcagagctgccctggcacagtacctgccctgtcggaggctctcactggcaaatgacagctctgtgcaaggagcactcccaagtataaaaattattacacagttttattctgaagaacattttgcattttaataaaaaaggatttatgtcaggaaagagtcatttacaaaccttgaagtgtttttgcctggatcagagtaagaatgtcttaagaagaggtttgtaaggtcttcataacaaagtggtgtttgttatttacaaa

SEQ ID NO: 38 - DNA, 454 bp, Homo sapiens
aaaagatcatcgcctctatcagacacttagggtcctggtctggcaattttggcctgatgtgatgccacaagacccaacagagagagacacagagtccaggataatgttgacagtggtgtagccctttaggagaaatggcgctccctgcggctggtattaggttaccattggcaccgaaggaaccaggaggataagaatatccataatttcagagctgccctggcacagtacctgccctgtcggaggctctcactggcaaatgacagctctgtgcaaggagcactcccaagtataaaaattattacacagttttattctgaagaacattttgcattttaataaaaaaggatttatgtcaggaaagagtcatttacaaaccttgaagtgtttttgcctggatcagagtaagaatgtcttaagaagaggtttgtaaggtcttcataacaaagtggtg

SEQ ID NO: 39 - DNA, 573 bp, Homo sapiens
tacaaagaaaaatttaatattcgatgagaggttgaaccaggcttaaagcagacatactaggaaatggtgcagcctgtaagaatgccagtttgtaagtactgactttggaaaagatcatcgcctctatcagacacttagggtcctggtctggcaattttggcctgatgtgatgccacaagacccaacagagagagacacagagtccaggataatgttgacagtggtgtagccctttaggagaaatggcgctccctgcggctggtattaggttaccattggcaccgaaggaaccaggaggataagaatatccataatttcagagctgccctggcacagtacctgccccgtcggaggctctcactggcaaatgacagctctgtgcaaggagcactcccaagtataaaaattattacacagttttattctgaagaacattttgcattttaataaaaaaggatttatgtcaggaaagagtcatttacaaaccttgaagtgtttttgcctggatcagagtaagaatgtcttaagaagaggtttgtaaggtcttcataacaaagtggtgtttgttattta

SEQ ID NO: 40 - DNA, 455 bp, Homo sapiens
  misc_feature (405)..(405): a or g or c or t/u
acaaagaaaaatttaatattcgatgagaggttgaaccaggcttaaagcagacatactaggaaatggtgcagcctgtaagaatgccagtttgtaagtactgactttggaaaagatcatcgcctctatcagacacttagggtcctggtctggcaattttggcctgatgtgatgccacaagacccaacagagagagacacagagtccaggataatgttgacagtggtgtagccctttaggagaaatggcgctccctgcggctggtattaggttaccattggcaccgaagaaccaggaggataagaatatccataatttcagagcttgccctggcacagtacctgccccgtcggaggctctcactgggcaaatggacagctctgtgcaaggagcactcccaagtataanaattattacacagttttattctgaagaacattttgcattttaataaaaaa

SEQ ID NO: 41 - DNA, 451 bp, Homo sapiens
tctatcagacacttagggtcctggtctggcaattttggcctgatgtgatgccacaagacccaacagagagagacacagagtccaggataatgttgacagtggtgtagccctttaggagaaatggcgctccctgcggctggtattaggttaccattggcaccgaaggaaccaggaggataagaatatccataatttcagagctgccctggcacagtacctgccccgtcggaggctctcactggcaaatgacagctctgtgcaaggagcactcccaagtataaaaattattacacagttttattctgaagaacattttgcattttaataaaaaaggatttatgtcaggaaagagtcatttacaaaccttgaagtgtttttgcctggatcagagtaagaatgtcttaagaagaggtttgtaaggtcttcataacaaagtggtgtttgttattta

SEQ ID NO: 42 - DNA, 526 bp, Homo sapiens
cagacatactaggaaatggtgcagcctgtaagaatgccagtttgtaagtactgactttggaaaagatcatcgcctctatcagacacttagggtcctggtctggcaattttggcctgatgtgatgccacaagacccaacagagagagacacagagtccaggataatgttgacagtggtgtagccctttaggagaaatggcgctccctgcggctggtattaggttaccattggcaccgaaggaaccaggaggataagaatatccataatttcagagctgccctggcacagtacctgccccgtcggaggctctcactggcaaatgacagctctgtgcaaggagcactcccaagtataaaaattattacacagttttattctgaagaacattttgcattttaataaaaaaaggatttatgtcaggaaagagtcatttacaaaccttgaagtgtttttgcctggatcagagtaagaatgtcttaagaagaggtttgtaaggtcttcataacaaaatggtgttagttattta

SEQ ID NO: 43 - DNA, 442 bp, Homo sapiens
gagtccaggataatgttgacagtggtgtagccctttaggagaaatggcgctccctgcggctggtattaggttaccattggcaccgaaggaaccaggaggataagaatatccataatttcagagctgccctggcacagtacctgccccgtcggaggctctcactggcaaatgacagctctgtgcaaggagcactcccaagtataaaaattattacacagttttattctgaagaacattttgcattttaataaaaaaggatttatgtcaggaaagagtcatttacaaaccttgaagtgtttttgcctggatcagagtaagaatgtcttaagaagaggtttgtaaggtcttcataacaaagtggtgtttgttatttacaaaaaaaaaaaaaaaaaaaattaacaggttgtctgtatactattaaaaattttggaccaaaaaaaaa

SEQ ID NO: 44 - DNA, 231 bp, Homo sapiens
gtcggaggctctcactggcaaatgacagctctgtgcaaggagcactcccaagtataaaaattattacacagttttattctgaagaacattttgcattttaataaaaaaggatttatgtcaggaaagagtcatttacaaaccttgaagtgtttttgcctggatcagagtaagaatgtcttaagaagaggtttgtaaggtcttcataacaaagtggtgtttgttatttacaaa

SEQ ID NO: 45 - DNA, 282 bp, Homo sapiens
  misc_feature (52)..(52): a or g or c or t/u
caggaggataagaatatccataattcagagctgccctggcacagtacctgcnccgtcggaggctctcactggcaaatgacagctctgtgcaaggagcactcccaagtataaaaattattacacagttttattctgaagaacattttgcattttaataaaaaaggatttatgtcaggaaagagtcatttacaaaccttgaagtgtttttgcctggatcagagtaagaatgtcttaagaagaggtttgtaaggtcttcataacaaagtggtgtttgttatttac

SEQ ID NO: 46 - DNA, 333 bp, Homo sapiens
agaaatggcgctccctgcggctggtattaggttaccatttgcaccgaaagaaccaggaggtaagaatatccatatttcagagctgccctggcacagtacctgcccgtcggaggctctcactggcaaatgacagctctgtgcaaggagcactcccaagtataaaaattattacacagttttattctgaagaacattttgcattttaataaaaaaggatttatgtcaggaaagagtcatttacaaaccttgaagtgtttttgcctggatcagagtaagaatgtcttaagaagaggtttgtaaggtcttcataacaaagtggtgtttgttatttac

SEQ ID NO: 47 - DNA, 376 bp, Homo sapiens
  misc_feature (31)..(31): a or g or c or t/u
ggctggtattaggttacccattggccaccgnaggaaccagggggataagaatttccataatttcagagctgccctggcacagtacctgccccgtcggaggctctcactggcaaatgacagctctgtgcaaggagcactcccaagtataaaaattattacacagttttattctgaagaacaattttgcnattttaataaaaaaggatttatgtcaggaaagagtcgatttacaaaccttgaagtgtttttgcctggatcagagtaagaatgtcttaagaagaggtttgtaaggtcttcataacaaagtggtgtttgttatttacaaaaaaaaaaaaaaaaaaaattaacaggttgtctgtatactattaaaaatttt

SEQ ID NO: 48 - DNA, 376 bp, Homo sapiens
gagtccaggataatgttgacagtggtgtagccctttaggagaaatggcgctccctgcggctggtattaggttaccattggcaccgaaggaaccaggaggataagaatatccataatttcagagctgccctggcacagtacctgccccgtcggaggctctcactggcaaatgacagctctgtgcaaggagcactcccaagtataaaaattattacacagttttattctgaagaacattttgcattttaataaaaaaggatttatgtcaggaaagagtcatttacaaaccttgaagtgtttttgcctggatcagagtaagaatgtcttaagaagaggtttgtaaggtcttcataaccaagtggtgtttgttatttaca

SEQ ID NO: 49 - DNA, 401 bp, Homo sapiens
aaatggcgctccctgcggctggtattaggttaccattggcaccgaaggaaccaggaggataagaatatccataatttcagagctgccctggcacagtacctgccccgtcggaggctctcactggcaaatgacagctctgtgcaaggagcactcccaagtataaaaattattacacagttttattctgaagaacattttgcattttaataaaaaaggatttatgtcaggaaagagtcatttacaaaccttgaagtgtttttgcctggatcagagtaagaatgtcttaagaagaggtttgtaaggtcttcataacaaagtggtgtttgttatttacaaaaaaaaaaaaaaaaaaaattaacaggttgtctgtatactattaaaaattttggaccaaaaaaaaa

SEQ ID NO: 50 - DNA, 443 bp, Homo sapiens
ccaggataatgttgacagtggtgtagccctttaggagaaatggcgctccctgcggctggtattaggttaccattggcaccgaaggaaccaggaggataagaatatccataatttcagagctgccctggcacagtacctgccccgtcggaggctctcactggcaaatgacagctctgtgcaaggagcactcccaagtataaaaattattacacagttttattctgaagaacattttgcattttaataaaaaaggatttatgtcaggaaaaagtcatttacaaaccttgaagtgtttttgcctggatcagagtaagaatgtcttaaaaagaggtttgtaaggtcttcataacaaagtggtgtttgttatttacaaaaaaaaaaaaaaaaaaaattaacaggttgtctgtatactattaaaaattttggcccaaaaaaaaaaaaaa

SEQ ID NO: 51 - DNA, 440 bp, Homo sapiens
ggataatgttgacagtggtgtagccctttaggagaaatggcgctccctgcggctggtattaggttaccattggcaccgaaggaaccaggaggataagaatatccataatttcagagctgccctggcacagtacctgccccgtcggaggctctcactggcaaatgacagctctgtgcaaggagcactcccaagtataaaaattattacacagttttattctgaagaacattttgcattttaataaaaaaggatttatgtcaggaaagagtcatttacaaaccttgaagtgtttttgcctggatcagagtaagaatgtcttaagaagaggtttgtaaggtcttcataacaaagtggtgtttgttatttacaaaaaaaaaaaaaaaaaaaattaacaggttgtctgtatactattaaaaattttggaccaaaaaaaaaaaaaa

SEQ ID NO: 52 - DNA, 292 bp, Homo sapiens
ctggcaaatgacagctctgtgcaaggagcactcccaagtataaaaattattacacagttttattctgaagaaccttttgcattttaataaaaaaggatttatgtcaggaaagagtcatttccaaaccttgaagtgtttttgcctggatcagagtaagaatgtcttaagaagaggtttgtaaggtcttcataacaaagtggtgtttgttatttccaaaaaaaaaaaaaaaaaaaattaacaggttgtctgtatactattaaaaattttggaccaaaaaaaaaaaaaaaaaaaa

SEQ ID NO: 53 - DNA, 729 bp, Artificial Sequence
  Consensus sequence
ggaaaaacatttcatcttaaaactttcctaagacaagggaaaacaaaaaaccatgctctacaacttcaaatttttcttacaaagaaaaatttaatattcgatgagaggttgaaccaggcttaaagcagacatactaggaaatggtgcagcctgtaagaatgccagtttgtaagtactgactttggaaaagatcatcgcctctatcagacacttagggtcctggtctggcaattttggcctgatgtgatgccacaagacccaacagagagagacacagagtccaggataatgttgacagtggtgtagccctttaggagaaatggcgctccctgcggctggtattaggttaccattggcaccgaaggaaccaggaggataagaatatccataatttcagagctgccctggcacagtacctgccccgtcggaggctctcactggcaaatgacagctctgtgcaaggagcactcccaagtataaaaattattacacagttttattctgaagaacattttgcattttaataaaaaaggatttatgtcaggaaagagtcatttacaaaccttgaagtgtttttgcctggatcagagtaagaatgtcttaagaagaggtttgtaaggtcttcataacaaagtggtgtttgttatttacaaaaaaaaaaaaaaaaaaaattaacaggttgtctgtatactattaaaaattttggaccaaaaaaaaaaaaaaaaaaaa

SEQ ID NO: 54 - DNA, 729 bp, Artificial Sequence; identical to SEQ ID NO: 53
  Assembled contig containing new 3′ end of CHDH

(19) United States
(12) Patent Application Publication
(10) Pub. No.: US 2006/0154267 A1
(43) Pub. Date: Jul. 13, 2006

(54) Diagnosis and treatment of breast cancer

(75) Inventors: Xiao-Jun Ma, San Diego, CA (US); Mark G. Erlander, Redwood City, CA (US); Dennis C. Sgroi, Winchester, MA (US); Edward Enright, San Diego, CA (US)

Correspondence Address: TOWNSEND AND TOWNSEND AND CREW, LLP, TWO EMBARCADERO CENTER, 8TH FLOOR, SAN FRANCISCO, CA, 94111, US

(73) Assignee: Arcturus Bioscience, Inc., Mountain View, CA (US)

(21) Appl. No.: 11/089,097

(22) Filed: Mar. 24, 2005

Related U.S. Application Data
(63) Continuation-in-part of application No. 10/773,761, filed on Feb. 6, 2004.
(63) Continuation-in-part of application No. 10/727,100, filed on Dec. 2, 2003.
(63) Continuation-in-part of application No. PCT/US04/30789, filed on Sep. 17, 2004.
(60) Provisional application No. 60/504,087, filed on Sep. 19, 2003.
(60) Provisional application No. 60/547,199, filed on Feb. 23, 2004.

(51) Int. Cl. G06Q 10/00; C12Q 1/68; G06Q 50/00

(52) U.S. Cl. 435/6; 705/2

(57) ABSTRACT

Methods and compositions are provided for the identification of expression signatures in ER+ breast cancer cases, where the signatures correlate with responsiveness, or lack thereof, to treatment with tamoxifen or another antiestrogen agent against breast cancer The signature profiles are identified based upon sampling of reference breast tissue samples from independent cases of breast cancer and provide a reliable set of molecular criteria for predicting the efficacy of treating a subject with breast cancer with tamoxifen or another antiestrogen agent against breast cancer. Additional methods and compositions are provided for predicting responsiveness to tamoxifen or another antiestrogen agent against breast cancer in cases of breast cancer by use of multiple biomarkers. Two biomarkers display increased expression correlated with tamoxifen response while two other biomarkers display decreased expression correlated with tamoxifen response.

### RELATED APPLICATIONS

This application is a continuation-in-part of PCT/US04/30789, filed Sep. 17, 2004, which claims benefit of priority from U.S. Provisional Patent Application 60/504,087 and 60/547,199, filed Sep. 19, 2003 and Feb. 23, 2004, respectively. This application is also a continuation-in-part of U.S. patent application Ser. Nos. 10/727,100 and 10/773,761, filed Dec. 2, 2003, and Feb. 6, 2004, respectively. All five applications are hereby incorporated by reference in their entireties as if fully set forth.

### FIELD OF THE INVENTION

The invention relates to the identification and use of gene expression profiles, or patterns, with clinical relevance to the treatment of breast cancer using tamoxifen (nolvadex) and other “antiestrogen” agents against breast cancer, including other “selective estrogen receptor modulators” (“SERM”s), “selective estrogen receptor downregulators” (“SERD”s), and aromatase inhibitors (“AI”s). In particular, the invention provides the identities of gene sequences the expression of which are correlated with patient survival and breast cancer recurrence in women treated with tamoxifen or other “antiestrogen” agents against breast cancer. The gene expression profiles, whether embodied in nucleic acid expression, protein expression, or other expression formats, may be used to select subjects afflicted with breast cancer who will likely respond positively to treatment with tamoxifen or another “antiestrogen” agent against breast cancer as well as those who will likely be non-responsive and thus candidates for other treatments. The invention also provides the identities of sets of sequences from multiple genes with expression patterns that are strongly predictive of responsiveness to tamoxifen and other “antiestrogen” agents against breast cancer.

### BACKGROUND OF THE INVENTION

Breast cancer is by far the most common cancer among women. Each year, more than 180,000 and 1 million women in the U.S. and worldwide, respectively, are diagnosed with breast cancer. Breast cancer is the leading cause of death for women between ages 50-55, and is the most common non-preventable malignancy in women in the Western Hemisphere. An estimated 2,167,000 women in the United States are currently living with the disease (National Cancer Institute, Surveillance Epidemiology and End Results (NCI SEER) program, Cancer Statistics Review (CSR), www-seer.ims.nci.nih.gov/Publications/CSR1973 (1998)). Based on cancer rates from 1995 through 1997, a report from the National Cancer Institute (NCI) estimates that about 1 in 8 women in the United States (approximately 12.8 percent) will develop breast cancer during her lifetime (NCI's Surveillance, Epidemiology, and End Results Program (SEER) publication SEER Cancer Statistics Review 1973-1997). Breast cancer is the second most common form of cancer, after skin cancer, among women in the United States. An estimated 250,100 new cases of breast cancer are expected to be diagnosed in the United States in 2001. Of these, 192,200 new cases of more advanced (invasive) breast cancer are expected to occur among women (an increase of 5% over last year), 46,400 new cases of early stage (in situ) breast cancer are expected to occur among women (up 9% from last year), and about 1,500 new cases of breast cancer are expected to be diagnosed in men (Cancer Facts & Figures 2001 American Cancer Society). An estimated 40,600 deaths (40,300 women, 400 men) from breast cancer are expected in 2001. Breast cancer ranks second only to lung cancer among causes of cancer deaths in women. Nearly 86% of women who are diagnosed with breast cancer are likely to still be alive five years later, though 24% of them will die of breast cancer after 10 years, and nearly half (47%) will die of breast cancer after 20 years.

Every woman is at risk for breast cancer. Over 70 percent of breast cancers occur in women who have no identifiable risk factors other than age (U.S. General Accounting Office. Breast Cancer, 1971-1991: Prevention, Treatment and Research. GAO/PEMD-92-12; 1991). Only 5 to 10% of breast cancers are linked to a family history of breast cancer (Henderson I C, Breast Cancer. In: Murphy G P, Lawrence W L, Lenhard R E (eds). Clinical Oncology. Atlanta, Ga.: American Cancer Society; 1995:198-219).

Each breast has 15 to 20 sections called lobes. Within each lobe are many smaller lobules. Lobules end in dozens of tiny bulbs that can produce milk. The lobes, lobules, and bulbs are all linked by thin tubes called ducts. These ducts lead to the nipple in the center of a dark area of skin called the areola. Fat surrounds the lobules and ducts. There are no muscles in the breast, but muscles lie under each breast and cover the ribs. Each breast also contains blood vessels and lymph vessels. The lymph vessels carry colorless fluid called lymph, and lead to the lymph nodes. Clusters of lymph nodes are found near the breast in the axilla (under the arm), above the collarbone, and in the chest.

Breast tumors can be either benign or malignant. Benign tumors are not cancerous, they do not spread to other parts of the body, and are not a threat to life. They can usually be removed, and in most cases, do not come back. Malignant tumors are cancerous, and can invade and damage nearby tissues and organs. Malignant tumor cells may metastasize, entering the bloodstream or lymphatic system. When breast cancer cells metastasize outside the breast, they are often found in the lymph nodes under the arm (axillary lymph nodes). If the cancer has reached these nodes, it means that cancer cells may have spread to other lymph nodes or other organs, such as bones, liver, or lungs.

Major and intensive research has been focused on early detection, treatment and prevention. This has included an emphasis on determining the presence of precancerous or cancerous ductal epithelial cells. These cells are analyzed, for example, for cell morphology, for protein markers, for nucleic acid markers, for chromosomal abnormalities, for biochemical markers, and for other characteristic changes that would signal the presence of cancerous or precancerous cells. This has led to various molecular alterations that have been reported in breast cancer, few of which have been well characterized in human clinical breast specimens. Molecular alterations include presence/absence of estrogen and progesterone steroid receptors, HER-2 expression/amplification (Mark H F, et al. HER-2/neu gene amplification in stages I-IV breast cancer detected by fluorescent in situ hybridization. Genet Med; 1(3):98-103 1999), Ki-67 (an antigen that is present in all stages of the cell cycle except GO and used as a marker for tumor cell proliferation, and prognostic markers (including oncogenes, tumor suppressor genes, and angiogenesis markers) like p53, p27, Cathepsin D, pS2, multi-drug resistance (MDR) gene, and CD31.

Tamoxifen is the antiestrogen agent most frequently prescribed in women with both early stage and metastatic hormone receptor-positive breast cancer (for reviews, see Clarke, R. et al. “Antiestrogen resistance in breast cancer and the role of estrogen receptor signaling.” Oncogene 22, 7316-39 (2003) and Jordan, C. “Historical perspective on hormonal therapy of advanced breast Cancer.” Clin. Ther. 24 Suppl A, A3-16 (2002)). In the adjuvant setting, tamoxifen therapy results in a 40-50% reduction in the annual risk of recurrence, leading to a 5.6% improvement in 10 year survival in lymph node negative patients, and a corresponding 10.9% improvement in node-positive patients (Group, E.B.C.T.C. Tamoxifen for early breast cancer. Cochrane Database Syst Rev, CD000486 (2001)). Tamoxifen is thought to act primarily as a competitive inhibitor of estrogen binding to estrogen receptor (ER). The absolute levels of ER expression, as well as that of the progesterone receptor (PR, an indicator of a functional ER pathway), are currently the best predictors of tamoxifen response in the clinical setting (Group, (2001) and Bardou, V. J. et al. “Progesterone receptor status significantly improves outcome prediction over estrogen receptor status alone for adjuvant endocrine therapy in two large breast cancer databases.” J Clin Oncol 21, 1973-9 (2003)).

However, 25% of ER+/PR+ tumors, 66% of ER+/PR− cases and 55% of ER−/PR+ cases fail to respond, or develop early resistance to tamoxifen, through mechanisms that remain largely unclear (see Clarke et al.; Nicholson, R. I. et al. “The biology of antihormone failure in breast cancer.” Breast Cancer Res Treat 80 Suppl 1, S29-34; discussion S35 (2003) and Osborne, C. K. et al. “Growth factor receptor cross-talk with estrogen receptor as a mechanism for tamoxifen resistance in breast cancer.” Breast 12, 362-7 (2003)). Currently, no reliable means exist to allow the identification of these non-responders. In these patients, the use of alternative hormonal therapies, such as the aromatase inhibitors letrozole and anastrozole (Ellis, M. J. et al. “Letrozole is more effective neoadjuvant endocrine therapy than tamoxifen for ErbB-1- and/or ErbB-2-positive, estrogen receptorpositive primary breast cancer: evidence from a phase III randomized trial.” J Clin Oncol 19, 3808-16 (2001); Buzdar, A. U. “Anastrozole: a new addition to the armamentarium against advanced breast cancer.” Am J Clin Oncol 21, 161-6 (1998); and Goss, P. E. et al. “A randomized trial of letrozole in postmenopausal women after five years of tamoxifen therapy for early-stage breast cancer.” N Engl J Med 349, 1793-802 (2003)); chemotherapeutic agents, or inhibitors of other signaling pathways, such as trastuzmab and gefitinib might offer the possibility of improving clinical outcome. Therefore, the ability to accurately predict tamoxifen treatment outcome should significantly advance the management of early stage breast cancer by identifying patients who are unlikely to benefit from TAM so that additional or alternative therapies may be sought.

Citation of documents herein is not intended as an admission that any is pertinent prior art. All statements as to the date or representation as to the contents of documents is based on the information available to the applicant and does not constitute any admission as to the correctness of the dates or contents of the documents.

### SUMMARY OF THE INVENTION

The present invention relates to the identification and use of gene expression patterns (or profiles or “signatures”) and the expression levels of individual gene sequences which are clinically relevant to breast cancer. In particular, the identities of genes that are correlated with patient survival and breast cancer recurrence (e.g. metastasis of the breast cancer) are provided. The gene expression profiles, whether embodied in nucleic acid expression, protein expression, or other expression formats, may be used to predict survival of subjects afflicted with breast cancer and the likelihood of breast cancer recurrence, including cancer metastasis.

The invention thus provides for the identification and use of gene expression patterns (or profiles or “signatures”) and the expression levels of individual gene sequences which correlate with (and thus are able to discriminate between) patients with good or poor survival outcomes. In one embodiment, the invention provides patterns that are able to distinguish patients with estrogen receptor (α isoform) positive (ER+) breast tumors into those with that are responsive, or likely to be responsive, to treatment with tamoxifen (TAM) or another “antiestrogen” agent against breast cancer (such as a “selective estrogen receptor modulator” (“SERM”), “selective estrogen receptor downregulator” (“SERD”), or aromatase inhibitor (“AI”)) and those that are non-responsive, or likely to be non-responsive, to such treatment. In an alternative embodiment, the invention may be applied to patients with breast tumors that do not display detectable levels of ER expression (so called “ER−” subjects) but where the patient will nonetheless benefit from application of the invention due to the presence of some low level of ER expression. Responsiveness may be viewed in terms of better survival outcomes over time. These patterns are thus able to distinguish patients with ER+ breast tumors into at least two subtypes.

In a first aspect, the present invention provides a non-subjective means for the identification of patients with breast cancer (ER+ or ER−) as likely to have a good or poor survival outcome following treatment with TAM or another “antiestrogen” agent against breast cancer by assaying for the expression patterns disclosed herein. Thus where subjective interpretation may have been previously used to determine the prognosis and/or treatment of breast cancer patients, the present invention provides objective gene expression patterns, which may used alone or in combination with subjective criteria to provide a more accurate assessment of ER+ or ER− breast cancer patient outcomes or expected outcomes, including survival and the recurrence of cancer, following treatment with TAM or another “antiestrogen” agent against breast cancer. The expression patterns of the invention thus provide a means to determine ER+ or ER− breast cancer prognosis as well as predict treatment outcome. Furthermore, the expression patterns can also be used as a means to assay small, node negative tumors that are not readily assayed by other means.

The gene expression patterns comprise one or more than one gene capable of discriminating between breast cancer outcomes with significant accuracy. The gene sequence(s) are identified as correlated with ER+ breast cancer outcomes such that the levels of their expression are relevant to a determination of the preferred treatment protocols for a patient, whether ER+ or ER−. Thus in one embodiment, the invention provides a method to determine the outcome of a subject afflicted with breast cancer by assaying a cell containing sample from said subject for expression of one or more than one gene disclosed herein as correlated with breast cancer outcomes following treatment with TAM or another “antiestrogen” agent against breast cancer.

The ability to correlate gene expression with breast cancer outcome and responsiveness to TAM is particularly advantageous in light of the possibility that up to 40% of ER+ subjects that undergo TAM treatment are non-responders. Therefore, the ability to identify, with confidence, these non-responders at an early time point permits the consideration and/or application of alternative therapies (such as a different “antiestrogen” agent against breast cancer or other anti-breast cancer treatments) to the non-responders. Stated differently, the ability to identify TAM non-responder subjects permits medical personnel to consider and/or utilize alternative therapies for the treatment of the subjects before time is spent on ineffective TAM therapy. Time spent on an ineffective therapy often permits further cancer growth, and the likelihood of success with alternative therapies diminishes over time given such growth. Therefore, the invention also provides methods to improve the survival outcome of non-responders by use of the methods disclosed herein to identify non-responders for treatment with alternative therapies.

Gene expression patterns of the invention are identified as described below. Generally, a large sampling of the gene expression profile of a sample is obtained through quantifying the expression levels of mRNA corresponding to many genes. This profile is then analyzed to identify genes, the expression of which are positively, or negatively, correlated, with ER+ breast cancer outcome upon treatment with TAM or another “antiestrogen” agent against breast cancer. An expression profile of a subset of human genes may then be identified by the methods of the present invention as correlated with a particular outcome. The use of multiple samples increases the confidence which a gene may be believed to be correlated with a particular survival outcome. Without sufficient confidence, it remains unpredictable whether expression of a particular gene is actually correlated with an outcome and also unpredictable whether expression of a particular gene may be successfully used to identify the outcome for a breast cancer patient. While the invention may be practiced based on the identities of the gene sequences disclosed herein or the actual sequences used independent of identification, the invention may also be practiced with any other sequences the expression of which is correlated with the expression of sequences disclosed herein. Such additional sequences may be identified by any means known in the art, including the methods disclosed herein.

A profile of genes that are highly correlated with one outcome relative to another may be used to assay an sample from a subject afflicted with breast cancer to predict the likely responsiveness (or lack thereof) to TAM or another “antiestrogen” agent against breast cancer in the subject from whom the sample was obtained. Such an assay may be used as part of a method to determine the therapeutic treatment for said subject based upon the breast cancer outcome identified.

As discussed below, the correlated genes may be used singly with significant accuracy or in combination to increase the ability to accurately correlating a molecular expression phenotype with a breast cancer outcome. This correlation is a way to molecularly provide for the determination of survival outcomes as disclosed herein. Additional uses of the correlated gene(s) are in the classification of cells and tissues; determination of diagnosis and/or prognosis; and determination and/or alteration of therapy.

The ability to discriminate is conferred by the identification of expression of the individual genes as relevant and not by the form of the assay used to determine the actual level of expression. An assay may utilize any identifying feature of an identified individual gene as disclosed herein as long as the assay reflects, quantitatively or qualitatively, expression of the gene in the “transcriptome” (the transcribed fraction of genes in a genome) or the “proteome” (the translated fraction of expressed genes in a genome). Additional assays include those based on the detection of polypeptide fragments of the relevant member or members of the proteome. Identifying features include, but are not limited to, unique nucleic acid sequences used to encode (DNA), or express (RNA), said gene or epitopes specific to, or activities of, a protein encoded by said gene. All that is required are the gene sequence(s) necessary to discriminate between breast cancer outcomes and an appropriate cell containing sample for use in an expression assay.

In another embodiment, the invention provides for the identification of the gene expression patterns by analyzing global, or near global, gene expression from single cells or homogenous cell populations which have been dissected away from, or otherwise isolated or purified from, contaminating cells beyond that possible by a simple biopsy. Because the expression of numerous genes fluctuate between cells from different patients as well as between cells from the same patient sample, multiple data from expression of individual genes and gene expression patterns are used as reference data to generate models which in turn permit the identification of individual gene(s), the expression of which are most highly correlated with particular breast cancer outcomes.

In additional embodiments, the invention provides physical and methodological means for detecting the expression of gene(s) identified by the models generated by individual expression patterns. These means may be directed to assaying one or more aspects of the DNA template(s) underlying the expression of the gene(s), of the RNA used as an intermediate to express the gene(s), or of the proteinaceous product expressed by the gene(s).

In further embodiments, the gene(s) identified by a model as capable of discriminating between breast cancer outcomes may be used to identify the cellular state of an unknown sample of cell(s) from the breast. Preferably, the sample is isolated via non-invasive means. The expression of said gene(s) in said unknown sample may be determined and compared to the expression of said gene(s) in reference data of gene expression patterns correlated with breast cancer outcomes. Optionally, the comparison to reference samples may be by comparison to the model(s) constructed based on the reference samples.

One advantage provided by the present invention is that contaminating, non-breast cells (such as infiltrating lymphocytes or other immune system cells) are not present to possibly affect the genes identified or the subsequent analysis of gene expression to identify the survival outcomes of patients with breast cancer. Such contamination is present where a biopsy is used to generate gene expression profiles. However, and as noted herein, the invention includes the identity of genes that may be used with significant accuracy even in the presence of contaminating cells.

In a second aspect, the invention provides a non-subjective means based on the expression of multiple genes, or combinations thereof, for the identification of patients with breast cancer as likely to have a good or poor survival outcome following treatment with TAM or another “antiestrogen” agent against breast cancer. These genes are members of the expression patterns disclosed herein which have been found to be strongly predictive of clinical outcome following TAM treatment of ER+ breast cancer.

The present invention thus provides gene sequences identified as differentially expressed in ER+ breast cancer in correlation to TAM responsiveness. The sequences of two genes display increased expression in ER+ breast cells that respond to TAM treatment (and thus lack of increased expression in nonresponsive cases). The sequences of two other genes display decreased expression in ER+ breast cells that respond to TAM treatment (and thus lack of decreased expression in nonresponsive cases).

The first set of sequences found to be more highly expressed in TAM responsive, ER+ breast cells are those of interleukin 17 receptor B (IL17RB), which has been mapped to human chromosome 3 at 3p21.1. IL17RB is also referred to as interleukin 17B receptor (IL17BR) and sequences corresponding to it, and thus may be used in the practice of the instant invention, are identified by UniGene Cluster Hs.5470.

The second set of sequences found to be more highly expressed in TAM responsive, ER+breast cells are those of a newly identified transcribed region of choline dehydrogenase (CHDH), which has been mapped to human chromosome 3 at 3p21.1. This is near the location mapped for the calcium channel, voltage-dependent, L type, alpha 1D subunit (CACNA1D) at 3p14.3. The invention is based in part on the unexpected discovery of an error in public databases that identified the sequence of AI240933 as corresponding to a transcribed part of CACNA1D (in Hs.399966). As detailed below, the transcribed regions of CHDH and CACNA1D are convergently oriented such that transcription proceeds from the regulatory regions of each toward the regulatory region of the other. Stated differently, they are convergently transcribed from complementary strands in the same region of chromosome 3.

Therefore, the invention includes the identification of AI240933 being in the wrong orientation with respect to CACNA1D transcription but in the correct orientation as CHDH transcription and as located at the 3′ end of CHDH transcription. Without being bound by theory, and offered to improve understanding of the invention, it is believed that the sequence of AI240933 is a part of the 3′ end of the CHDH transcript. It is possibly part of the 3′ untranslated region (UTR) of CHDH. The invention may be practiced with sequences corresponding to CHDH as well as those identified by Hs.126688.

The first set of sequences found to be expressed at lower levels in TAM responsive, ER+breast cells are those of homeobox B113 (HOXB13), which has been mapped to human chromosome 17 at 17q21.2. Sequences corresponding to HOXB13, and thus may be used in the practice of the instant invention, are identified by UniGene Cluster Hs.66731.

The second set of sequences found to be expressed at lower levels in TAM responsive, ER+ breast cells are those of quinolinate phosphoribosyltransferase (QPRT, also known as nicotinate-nucleotide pyrophosphorylase, carboxylating), which has been mapped to human chromosome 16 at 16p12.1. Sequences corresponding to QPRT may be used in the practice of the instant invention, are identified by UniGene Cluster Hs.335116.

While the invention may be practiced based on the identities of these gene sequences or the actual sequences used independent of the assigned identity, the invention may also be practiced with any other sequence the expression of which is correlated with the expression of these disclosed sequences. Such additional sequences may be identified by any means known in the art, including the methods disclosed herein.

The identified sequences may thus be used in methods of determining the responsiveness, or non-responsiveness, of a subject's ER+ or ER− breast cancer to TAM treatment, or treatment with another “antiestrogen” agent against breast cancer, via analysis of breast cells in a tissue or cell containing sample from a subject. As non-limiting examples, the lack of increased expression of IL17BR and/or CHDH sequences and/or the lack of decreased expression of HOXB13 and/or QPRT sequences may be used as an indicator of nonresponsive cases. The present invention provides a non-empirical means for determining responsiveness to TAM or another SERM in ER+ or ER− patients. This provides advantages over the use of a “wait and see” approach following treatment with TAM or other “antiestrogen” agent against breast cancer. The expression levels of these sequences may also be used as a means to assay small, node negative tumors that are not readily assessed by conventional means.

The expression levels of the identified sequences may be used alone or in combination with other sequences capable of determining responsiveness to treatment with TAM or another “antiestrogen” agent against breast cancer. Preferably, the sequences of the invention are used alone or in combination with each other, such as in the format of a ratio of expression levels that can have improved predictive power over analysis based on expression of sequences corresponding to individual genes. The invention provides for ratios of the expression level of a sequence that is underexpressed to the expression level of a sequence that is overexpressed as a indicator of responsiveness or non-responsiveness. For example, ratios of either HOXB13 or QPRT relative to IL17BR or CHDH may be used. Of course the ratios of either IL17BR or CHDH relative to either HOXB13 or QPRT may also be used.

The present invention provides means for correlating a molecular expression phenotype with a physiological response in a subject with ER+ or ER− breast cancer. This correlation provides a way to molecularly diagnose and/or determine treatment for a breast cancer afflicted subject. Additional uses of the sequences are in the classification of cells and tissues; and determination of diagnosis and/or prognosis. Use of the sequences to identify cells of a sample as responsive, or not, to treatment with TAM or other “antiestrogen” agent against breast cancer may be used to determine the choice, or alteration, of therapy used to treat such cells in the subject, as well as the subject itself, from which the sample originated.

Such methods of the invention may be used to assist the determination of providing tamoxifen or another “antiestrogen” agent against breast cancer as a chemopreventive or chemoprotective agent to a subject at high risk for development of breast cancer. These methods of the invention are an advance over the studies of Fabian et al. (J Natl Cancer Inst. 92(15):1217-27, 2000), which proposed a combination of cytomorphology and the Gail risk model to identify high risk patients. The methods may be used in combination with assessments of relative risk of breast cancer such as that discussed by Tan-Chiu et al. (J Natl Cancer Inst. 95(4):302-307, 2003). Non-limiting examples include assaying of minimally invasive sampling, such as random (periareolar) fine needle aspirates or ductal lavage samples (such as that described by Fabian et al. and optionally in combination with or as an addition to a mammogram positive for benign or malignant breast cancer), of breast cells for the expression levels of gene sequences as disclosed herein to assist in the determination of administering therapy with an “antiestrogen” agent against breast cancer, such as that which may occur in cases of high risk subjects (like those described by Tan-Chiu et al.). The assays would thus lead to the identification of subjects for who the application of an “antiestrogen” agent against breast cancer would likely be beneficial as a chemopreventive or chemoprotective agent. It is contemplated that such application as enabled by the instant invention could lead to beneficial effects such as those seen with the administration of tamoxifen (see for example, Wickerham D. L., Breast Cancer Res. and Treatment 75 Suppl 1:S7-12, Discussion S33-5, 2000). Other applications of the invention include assaying of advanced breast cancer, including metastatic cancer, to determine the responsiveness, or non-responsiveness, thereof to treatment with an “antiestrogen” agent against breast cancer.

An assay of the invention may utilize a means related to the expression level of the sequences disclosed herein as long as the assay reflects, quantitatively or qualitatively, expression of the sequence. Preferably, however, a quantitative assay means is preferred. The ability to determine responsiveness to TAM or other “antiestrogen” agent against breast cancer and thus outcome of treatment therewith is provided by the recognition of the relevancy of the level of expression of the identified sequences and not by the form of the assay used to determine the actual level of expression. Identifying features of the sequences include, but are not limited to, unique nucleic acid sequences used to encode (DNA), or express (RNA), the disclosed sequences or epitopes specific to, or activities of, proteins encoded by the sequences. Alternative means include detection of nucleic acid amplification as indicative of increased expression levels and nucleic acid inactivation, deletion, or methylation, as indicative of decreased expression levels. Stated differently, the invention may be practiced by assaying one or more aspect of the DNA template(s) underlying the expression of the disclosed sequence(s), of the RNA used as an intermediate to express the sequence(s), or of the proteinaceous product expressed by the sequence(s), as well as proteolytic fragments of such products. As such, the detection of the presence of, amount of, stability of, or degradation (including rate) of, such DNA, RNA and proteinaceous molecules may be used in the practice of the invention.

The practice of the present invention is unaffected by the presence of minor mismatches between the disclosed sequences and those expressed by cells of a subject's sample. A non-limiting example of the existence of such mismatches are seen in cases of sequence polymorphisms between individuals of a species, such as individual human patients within Homo sapiens. Knowledge that expression of the disclosed sequences (and sequences that vary due to minor mismatches) is correlated with the presence of non-normal or abnormal breast cells and breast cancer is sufficient for the practice of the invention with an appropriate cell containing sample via an assay for expression.

In one embodiment, the invention provides for the identification of the expression levels of the disclosed sequences by analysis of their expression in a sample containing ER+ or ER− breast cells. In one preferred embodiment, the sample contains single cells or homogenous cell populations which have been dissected away from, or otherwise isolated or purified from, contaminating cells beyond that possible by a simple biopsy. Alternatively, undissected cells within a “section” of tissue may be used. Multiple means for such analysis are available, including detection of expression within an assay for global, or near global, gene expression in a sample (e.g. as part of a gene expression profiling analysis such as on a microarray) or by specific detection, such as quantitative PCR (Q-PCR), or real time, quantitative PCR.

Preferably, the sample is isolated via non-invasive or minimally invasive means. The expression of the disclosed sequence(s) in the sample may be determined and compared to the expression of said sequence(s) in reference data of non-normal or cancerous breast cells. Alternatively, the expression level may be compared to expression levels in normal or non-cancerous cells, preferably from the same sample or subject. In embodiments of the invention utilizing Q-PCR, the expression level may be compared to expression levels of reference genes in the same sample or a ratio of expression levels may be used.

When individual breast cells are isolated in the practice of the invention, one benefit is that contaminating, non-breast cells (such as infiltrating lymphocytes or other immune system cells) are not present to possibly affect detection of expression of the disclosed sequence(s). Such contamination is present where a biopsy is used to generate gene expression profiles. However, analysis of differential gene expression and correlation to ER+ breast cancer outcomes with both isolated and non-isolated samples, as described herein, increases the confidence level of the disclosed sequences as capable of having significant predictive power with either type of sample.

While the present invention is described mainly in the context of human breast cancer, it may be practiced in the context of breast cancer of any animal known to be potentially afflicted by breast cancer. Preferred animals for the application of the present invention are mammals, particularly those important to agricultural applications (such as, but not limited to, cattle, sheep, horses, and other “farm animals”), animal models of breast cancer, and animals for human companionship (such as, but not limited to, dogs and cats).

The above aspects and embodiments of the invention may be applied equally with respect to use of more than one “antiestrogen” agent against breast cancer. In the case of a combination of agents, any combination of more than one SERM, SERD, or AI may be used in place of TAM or another “antiestrogen” agent against breast cancer. Aromatase is an enzyme that provides a major source of estrogen in body tissues including the breast, liver, muscle and fat. Without being bound by theory, and solely provided to assist in a better understanding of the invention, AIs are understood to function in a manner comparable to TAM and other “antiestrogen” agents against breast cancer, which are thought to act as antagonists of estrogen receptor in breast tissues and thus as against breast cancer. AIs may be either nonsteroidal or steroidal agents. Examples of the former, which inhibit aromatase via the heme prosthetic group) include, but are not limited to, anastrozole (arimidex), letrozole (femara), and vorozole (rivisor), which have been used or contemplated as treatments for metastatic breast cancer. Examples of steroidal AIs, which inactivate aromatase, include, but are not limited to, exemestane (aromasin), androstenedione, and formestane (lentaron).

Other forms of therapy to reduce estrogen levels include surgical or chemical ovarian ablation. The former is physical removal of the ovaries while the latter is the use of agents to block ovarian production of estrogen. One non-limiting example of the latter are agonists of gonadotropin releasing hormone (GnRH), such as goserelin (zoladex). Of course the instant invention may also be practiced with these therapies in place of treatment with one or more “antiestrogen” agent against breast cancer.

The invention disclosed herein is based in part on the performance of a genome-wide microarray analysis of hormone receptor-positive invasive breast tumors from 60 patients treated with adjuvant tamoxifen alone, leading to the identification of a two-gene expression ratio that is highly predictive of clinical outcome. This expression ratio, which is readily adapted to PCR-based analysis of standard paraffin-embedded clinical specimens, was validated in an independent set of 20 patients as described below.

### BRIEF DESCRIPTION OF THE DRAWINGS

FIG. 1 shows receiver operating characteristic (ROC) analyses of IL17BR, HOXB13, and CACNA1D expression levels as predictors of breast cancer outcomes in whole tissue sections (top 3 graphs) and laser microdissected cells (bottom 3 graphs). AUC refers to area under the curve.

FIG. 2 contains six parts relating to the validation of a ratio of HOXB13 expression to IL17BR expression as an indicator of responsiveness, or lack thereof, to TAM. Parts a and b show the results of gene expression analysis of HOXB13 and IL17BR sequences by Q-PCR in both Responder and Non-responder samples. Plots of the Responder and Non-responder training and validation data sets are shown in Parts c and d, where “0” indicates Responder datapoints in both and “1” indicates Non-responder datapoints in both. Parts e and f show plots of the Responder and Non-responder training and validation data sets as a function of survival, where the upper line in each Part represents the Responders and the lower line represents the Non-responders.

FIG. 3 shows a schematic representation of the known 3′ region of the CHDH gene sequence in combination with additional CHDH 3′ untranslated sequences identified by the instant invention.

FIG. 4 shows the results of a PCR amplification reaction wherein an amplicon consistent with that expected from the schematic of FIG. 1 is produced. The PCR primers used were as follows: forward CHDH primer: 5′-AAAGTCTTGGGAAATGAGACAAGT-3′; reverse primers 83R: 5′-AGCTGTCATTTGCCAGTGAGA-3′ and 81R: 5′-CTGTCATTTGCCAGTGAGAGC-3′.

FIG. 5 shows the alignment of 28 sequences to identify a contig comprising the CHDH 3′ end region. The alignment includes the sequence of AI240933, which includes the 3′ end of the assembled consensus sequence.

FIG. 6 shows the sequence of an assembled contig containing the new 3′ end of CHDH.

FIG. 7 shows a representation of a region of human chromosome 3 wherein the location of CACNA1D is identified via “Hs.399966” and the location of CHDH is identified via “Hs.126688”.

FIG. 8, Part A contains six parts relating to the validation of a ratio of QPRT expression to CHDH expression as an indicator of responsiveness, or lack thereof, to TAM. The three portions identified by “QPRT:CHDH AI240933” reflect the ratio using a probe for expression of the GenBank AI240933 sequence. The three portions identified by “QPRT:CHDH AJ272267” reflect the ratio using a probe for expression of the GenBank AJ272267 sequence, identified as that of a partial mRNA for CHDH. Part B contains analogous use of a ratio of HOXB13 expression to IL17BR expression as an indicator of TAM responsiveness. Plots of the Responder (“R”) and Non-responder (“NR”) data sets are shown. P values are two-sample t-test.

### MODES OF PRACTICING THE INVENTION

Definitions of terms as used herein:

A gene expression “pattern” or “profile” or “signature” refers to the relative expression of genes correlated with responsiveness to treatment of ER+breast cancer with TAM or another “antiestrogen” agent against breast cancer. Responsiveness or lack thereof may be expressed as survival outcomes which are correlated with an expression “pattern” or “profile” or “signature” that is able to distinguish between, and predict, said outcomes.

A “selective estrogen receptor modulator” or SERM is an “antiestrogen” agent that in some tissues act like estrogens (agonist) but block estrogen action in other tissues (antagonist). A “selective estrogen receptor downregulators” (or “SERD”s) or “pure” antiestrogens includes agents which block estrogen activity in all tissues. See Howell et al. (Best Bractice & Res. Clin. Endocrinol. Metab. 18(1):47-66, 2004). Preferred SERMs of the invention are those that are antagonists of estrogen in breast tissues and cells, including those of breast cancer. Non-limiting examples of such include TAM, raloxifene, GW5638, and ICI 182,780. The possible mechanisms of action by various SERMs have been reviewed (see for example Jordan et al., 2003, Breast Cancer Res. 5:281-283; Hall et al., 2001, J. Biol. Chem. 276(40):36869-36872; Dutertre et al. 2000, J. Pharmacol. Exp. Therap. 295(2):431-437; and Wijayaratne et al., 1999, Endocrinology 140(12):5828-5840). Other non-limiting examples of SERMs in the context of the invention include triphenylethylenes, such as tamoxifen, GW5638, TAT-59, clomiphene, toremifene, droloxifene, and idoxifene; benzothiophenes, such as arzoxiphene (LY353381 or LY353381-HCl); benzopyrans, such as EM-800; naphthalenes, such as CP-336,156; and ERA-923.

Non-limiting examples of SERD or “pure” antiestrogens include agents such as ICI 182,780 (fulvestrant or faslodex) or the oral analogue SR16243 and ZK 191703 as well as aromatase inhibitors and chemical ovarian ablation agents as described herein.

Other agents encompassed by SERM as used herein include progesterone receptor inhibitors and related drugs, such as progestomimetics like medroxyprogesterone acetate, megace, and RU-486; and peptide based inhibitors of ER action, such as LH-RH analogs (leuprolide, zoladex, [D-Trp6]LH-RH), somatostatin analogs, and LXXLL motif mimics of ER as well as tibolone and resveratrol. As noted above, preferred SERMs of the invention are those that are antagonist of estrogen in breast tissues and cells, including those of breast cancer. Non-limiting examples of preferred SERMs include the actual or contemplated metabolites (in vivo) of any SERM, such as, but not limited to, 4-hydroxytamoxifen (metabolite of tamoxifen), EM652 (or SCH 57068 where EM-800 is a prodrug of EM-652), and GW7604 (metabolite of GW5638). See Willson et al. (1997, Endocrinology 138(9):3901-3911) and Dauvois et al. (1992, Proc. Nat'l. Acad. Sci., USA 89:4037-4041) for discussions of some specific SERMs.

Other preferred SERMs are those that produce the same relevant gene expression profile as tamoxifen or 4-hydroxytamoxifen. One example of means to identify such SERMs is provided by Levenson et al. (2002, Cancer Res. 62:4419-4426).

A “gene” is a polynucleotide that encodes a discrete product, whether RNA or proteinaceous in nature. It is appreciated that more than one polynucleotide may be capable of encoding a discrete product. The term includes alleles and polymorphisms of a gene that encodes the same product, or a functionally associated (including gain, loss, or modulation of function) analog thereof, based upon chromosomal location and ability to recombine during normal mitosis.

A “sequence” or “gene sequence” as used herein is a nucleic acid molecule or polynucleotide composed of a discrete order of nucleotide bases. The term includes the ordering of bases that encodes a discrete product (i.e. “coding region”), whether RNA or proteinaceous in nature, as well as the ordered bases that precede or follow a “coding region”. Non-limiting examples of the latter include 5′ and 3′ untranslated regions of a gene. It is appreciated that more than one polynucleotide may be capable of encoding a discrete product. It is also appreciated that alleles and polymorphisms of the disclosed sequences may exist and may be used in the practice of the invention to identify the expression level(s) of the disclosed sequences or the allele or polymorphism. Identification of an allele or polymorphism depends in part upon chromosomal location and ability to recombine during mitosis.

The terms “correlate” or “correlation” or equivalents thereof refer to an association between expression of one or more genes and a physiological response of a breast cancer cell and/or a breast cancer patient in comparison to the lack of the response. A gene may be expressed at higher or lower levels and still be correlated with responsiveness, non-responsiveness or breast cancer survival or outcome. The invention provides, for example, for the correlation between increases in expression of IL17BR and/or CHDH sequences and responsiveness of ER+breast cells to TAM or another “antiestrogen” agent against breast cancer. Thus increases are indicative of responsiveness. Conversely, the lack of increases, including unchanged expression levels, are indicators of non-responsiveness. Similarly, the invention provides, for example, for the correlation between decreases in expression of HOXB13 and/or QPRT sequences and responsiveness of ER+ breast cells to TAM or another SERM. Thus decreases are indicative of responsiveness while the lack of decreases, including unchanged expression levels, are indicators of non-responsiveness. Increases and decreases may be readily expressed in the form of a ratio between expression in a non-normal cell and a normal cell such that a ratio of one (1) indicates no difference while ratios of two (2) and one-half indicate twice as much, and half as much, expression in the non-normal cell versus the normal cell, respectively. Expression levels can be readily determined by quantitative methods as described below.

For example, increases in gene expression can be indicated by ratios of or about 1.1, of or about 1.2, of or about 1.3, of or about 1.4, of or about 1.5, of or about 1.6, of or about 1.7, of or about 1.8, of or about 1.9, of or about 2, of or about 2.5, of or about 3, of or about 3.5, of or about 4, of or about 4.5, of or about 5, of or about 5.5, of or about 6, of or about 6.5, of or about 7, of or about 7.5, of or about 8, of or about 8.5, of or about 9, of or about 9.5, of or about 10, of or about 15, of or about 20, of or about 30, of or about 40, of or about 50, of or about 60, of or about 70, of or about 80, of or about 90, of or about 100, of or about 150, of or about 200, of or about 300, of or about 400, of or about 500, of or about 600, of or about 700, of or about 800, of or about 900, or of or about 1000. A ratio of 2 is a 100% (or a two-fold) increase in expression. Decreases in gene expression can be indicated by ratios of or about 0.9, of or about 0.8, of or about 0.7, of or about 0.6, of or about 0.5, of or about 0.4, of or about 0.3, of or about 0.2, of or about 0.1, of or about 0.05, of or about 0.01, of or about 0.005, of or about 0.001, of or about 0.0005, of or about 0.0001, of or about 0.00005, of or about 0.00001, of or about 0.000005, or of or about 0.000001.

For a given phenotype, a ratio of the expression of a gene sequence expressed at increased levels in correlation with the phenotype to the expression of a gene sequence expressed at decreased levels in correlation with the phenotype may also be used as an indicator of the phenotype. As a non-limiting example, the phenotype of non-responsiveness to tamoxifen treatment of breast cancer is correlated with increased expression of HOXB13 and/or QPRT as well as decreased expression of IL17BR and/or CHDH. Therefore, a ratio of the expression levels of HOXB13 or QPRT to IL17BR or CHDH may be used as an indicator of non-responsiveness.

A “polynucleotide” is a polymeric form of nucleotides of any length, either ribonucleotides or deoxyribonucleotides. This term refers only to the primary structure of the molecule. Thus, this term includes double- and single-stranded DNA and RNA. It also includes known types of modifications including labels known in the art, methylation, “caps”, substitution of one or more of the naturally occurring nucleotides with an analog, and internucleotide modifications such as uncharged linkages (e.g., phosphorothioates, phosphorodithioates, etc.), as well as unmodified forms of the polynucleotide.

The term “amplify” is used in the broad sense to mean creating an amplification product can be made enzymatically with DNA or RNA polymerases. “Amplification,” as used herein, generally refers to the process of producing multiple copies of a desired sequence, particularly those of a sample. “Multiple copies” mean at least 2 copies. A “copy” does not necessarily mean perfect sequence complementarity or identity to the template sequence. Methods for amplifying mRNA are generally known in the art, and include reverse transcription PCR (RT-PCR) and those described in U.S. patent application Ser. No. 10/062,857 (filed on Oct. 25, 2001), as well as U.S. Provisional Patent Application 60/298,847 (filed Jun. 15, 2001) and 60/257,801 (filed Dec. 22, 2000), all of which are hereby incorporated by reference in their entireties as if fully set forth. Another method which may be used is quantitative PCR (or Q-PCR). Alternatively, RNA may be directly labeled as the corresponding cDNA by methods known in the art.

By “corresponding”, it is meant that a nucleic acid molecule shares a substantial amount of sequence identity with another nucleic acid molecule. Substantial amount means at least 95%, usually at least 98% and more usually at least 99%, and sequence identity is determined using the BLAST algorithm, as described in Altschul et al. (1990), J. Mol. Biol. 215:403-410 (using the published default setting, i.e. parameters w=4, t=17).

A “microarray” is a linear or two-dimensional or three dimensional (and solid phase) array of preferably discrete regions, each having a defined area, formed on the surface of a solid support such as, but not limited to, glass, plastic, or synthetic membrane. The density of the discrete regions on a microarray is determined by the total numbers of immobilized polynucleotides to be detected on the surface of a single solid phase support, preferably at least about 50/cm², more preferably at least about 100/cm², even more preferably at least about 500/cm², but preferably below about 1,000/cm². Preferably, the arrays contain less than about 500, about 1000, about 1500, about 2000, about 2500, or about 3000 immobilized polynucleotides in total. As used herein, a DNA microarray is an array of oligonucleotides or polynucleotides placed on a chip or other surfaces used to hybridize to amplified or cloned polynucleotides from a sample. Since the position of each particular group of primers in the array is known, the identities of a sample polynucleotides can be determined based on their binding to a particular position in the microarray. As an alternative to the use of a microarray, an array of any size may be used in the practice of the invention, including an arrangement of one or more position of a two-dimensional or three dimensional arrangement in a solid phase to detect expression of a single gene sequence.

Because the invention relies upon the identification of genes that are over- or under-expressed, one embodiment of the invention involves determining expression by hybridization of mRNA, or an amplified or cloned version thereof, of a sample cell to a polynucleotide that is unique to a particular gene sequence. Preferred polynucleotides of this type contain at least about 16, at least about 18, at least about 20, at least about 22, at least about 24, at least about 26, at least about 28, at least about 30, or at least about 32 consecutive basepairs of a gene sequence that is not found in other gene sequences. The term “about” as used in the previous sentence refers to an increase or decrease of 1 from the stated numerical value. Even more preferred are polynucleotides of at least or about 50, at least or about 100, at least about or 150, at least or about 200, at least or about 250, at least or about 300, at least or about 350, at least or about 400, at least or about 450, or at least or about 500 consecutive bases of a sequence that is not found in other gene sequences. The term “about” as used in the preceding sentence refers to an increase or decrease of 10% from the stated numerical value. Longer polynucleotides may of course contain minor mismatches (e.g. via the presence of mutations) which do not affect hybridization to the nucleic acids of a sample. Such polynucleotides may also be referred to as polynucleotide probes that are capable of hybridizing to sequences of the genes, or unique portions thereof, described herein. Such polynucleotides may be labeled to assist in their detection. Preferably, the sequences are those of mRNA encoded by the genes, the corresponding cDNA to such mRNAs, and/or amplified versions of such sequences. In preferred embodiments of the invention, the polynucleotide probes are immobilized on an array, other solid support devices, or in individual spots that localize the probes.

In another embodiment of the invention, all or part of a disclosed sequence may be amplified and detected by methods such as the polymerase chain reaction (PCR) and variations thereof, such as, but not limited to, quantitative PCR (Q-PCR), reverse transcription PCR (RT-PCR), and real-time PCR (including as a means of measuring the initial amounts of mRNA copies for each sequence in a sample), optionally real-time RT-PCR or real-time Q-PCR. Such methods would utilize one or two primers that are complementary to portions of a disclosed sequence, where the primers are used to prime nucleic acid synthesis. The newly synthesized nucleic acids are optionally labeled and may be detected directly or by hybridization to a polynucleotide of the invention. The newly synthesized nucleic acids may be contacted with polynucleotides (containing sequences) of the invention under conditions which allow for their hybridization. Additional methods to detect the expression of expressed nucleic acids include RNAse protection assays, including liquid phase hybridizations, and in situ hybridization of cells.

Alternatively, and in yet another embodiment of the invention, gene expression may be determined by analysis of expressed protein in a cell sample of interest by use of one or more antibodies specific for one or more epitopes of individual gene products (proteins), or proteolytic fragments thereof, in said cell sample or in a bodily fluid of a subject. The cell sample may be one of breast cancer epithelial cells enriched from the blood of a subject, such as by use of labeled antibodies against cell surface markers followed by fluorescence activated cell sorting (FACS). Such antibodies are preferably labeled to permit their easy detection after binding to the gene product. Detection methodologies suitable for use in the practice of the invention include, but are not limited to, immunohistochemistry of cell containing samples or tissue, enzyme linked immunosorbent assays (ELISAs) including antibody sandwich assays of cell containing tissues or blood samples, mass spectroscopy, and immuno-PCR.

The term “label” refers to a composition capable of producing a detectable signal indicative of the presence of the labeled molecule. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

The term “support” refers to conventional supports such as beads, particles, dipsticks, fibers, filters, membranes and silane or silicate supports such as glass slides.

As used herein, a “breast tissue sample” or “breast cell sample” refers to a sample of breast tissue or fluid isolated from an individual suspected of being afflicted with, or at risk of developing, breast cancer. Such samples are primary isolates (in contrast to cultured cells) and may be collected by any non-invasive or minimally invasive means, including, but not limited to, ductal lavage, fine needle aspiration, needle biopsy, the devices and methods described in U.S. Pat. No. 6,328,709, or any other suitable means recognized in the art. Alternatively, the “sample” may be collected by an invasive method, including, but not limited to, surgical biopsy.

“Expression” and “gene expression” include transcription and/or translation of nucleic acid material.

As used herein, the term “comprising” and its cognates are used in their inclusive sense; that is, equivalent to the term “including” and its corresponding cognates.

Conditions that “allow” an event to occur or conditions that are “suitable” for an event to occur, such as hybridization, strand extension, and the like, or “suitable” conditions are conditions that do not prevent such events from occurring. Thus, these conditions permit, enhance, facilitate, and/or are conducive to the event. Such conditions, known in the art and described herein, depend upon, for example, the nature of the nucleotide sequence, temperature, and buffer conditions. These conditions also depend on what event is desired, such as hybridization, cleavage, strand extension or transcription.

Sequence “mutation,” as used herein, refers to any sequence alteration in the sequence of a gene disclosed herein interest in comparison to a reference sequence. A sequence mutation includes single nucleotide changes, or alterations of more than one nucleotide in a sequence, due to mechanisms such as substitution, deletion or insertion. Single nucleotide polymorphism (SNP) is also a sequence mutation as used herein. Because the present invention is based on the relative level of gene expression, mutations in non-coding regions of genes as disclosed herein may also be assayed in the practice of the invention.

“Detection” includes any means of detecting, including direct and indirect detection of gene expression and changes therein. For example, “detectably less” products may be observed directly or indirectly, and the term indicates any reduction (including the absence of detectable signal). Similarly, “detectably more” product means any increase, whether observed directly or indirectly.

Increases and decreases in expression of the disclosed sequences are defined in the following terms based upon percent or fold changes over expression in normal cells. Increases may be of 10, 20, 30, 40, 50, 60, 70, 80, 90, 100, 120, 140, 160, 180, or 200% relative to expression levels in normal cells. Alternatively, fold increases may be of 1, 1.5, 2, 2.5, 3, 3.5, 4, 4.5, 5, 5.5, 6, 6.5, 7, 7.5, 8, 8.5, 9, 9.5, or 10 fold over expression levels in normal cells. Decreases may be of 10, 20, 30, 40, 50, 55, 60, 65, 70, 75, 80, 82, 84, 86, 88, 90, 92, 94, 96, 98, 99 or 100% relative to expression levels in normal cells.

Unless defined otherwise all technical and scientific terms used herein have the same meaning as commonly understood to one of ordinary skill in the art to which this invention belongs.

### EMBODIMENTS OF THE INVENTION

In a first aspect, the disclosed invention relates to the identification and use of gene expression patterns (or profiles or “signatures”) which discriminate between (or are correlated with) breast cancer survival in a subject treated with tamoxifen (TAM) or another “antiestrogen” agent against breast cancer. Such patterns may be determined by the methods of the invention by use of a number of reference cell or tissue samples, such as those reviewed by a pathologist of ordinary skill in the pathology of breast cancer, which reflect breast cancer cells as opposed to normal or other non-cancerous cells. The outcomes experienced by the subjects from whom the samples may be correlated with expression data to identify patterns that correlate with the outcomes following treatment with TAM or another “antiestrogen” agent against breast cancer. Because the overall gene expression profile differs from person to person, cancer to cancer, and cancer cell to cancer cell, correlations between certain cells and genes expressed or underexpressed may be made as disclosed herein to identify genes that are capable of discriminating between breast cancer outcomes.

The present invention may be practiced with any number of the genes believed, or likely to be, differentially expressed with respect to breast cancer outcomes, particularly in cases of ER+ breast cancer. The identification may be made by using expression profiles of various homogenous breast cancer cell populations, which were isolated by microdissection, such as, but not limited to, laser capture microdissection (LCM) of 100-1000 cells. The expression level of each gene of the expression profile may be correlated with a particular outcome. Alternatively, the expression levels of multiple genes may be clustered to identify correlations with particular outcomes.

Genes with significant correlations to breast cancer survival when the subject is treated with tamoxifen may be used to generate models of gene expressions that would maximally discriminate between outcomes where a subject responds to treatment with tamoxifen or another “antiestrogen” agent against breast cancer and outcomes where the treatment is not successful. Alternatively, genes with significant correlations may be used in combination with genes with lower correlations without significant loss of ability to discriminate between outcomes. Such models may be generated by any appropriate means recognized in the art, including, but not limited to, cluster analysis, supported vector machines, neural networks or other algorithm known in the art. The models are capable of predicting the classification of a unknown sample based upon the expression of the genes used for discrimination in the models. “Leave one out” cross-validation may be used to test the performance of various models and to help identify weights (genes) that are uninformative or detrimental to the predictive ability of the models. Cross-validation may also be used to identify genes that enhance the predictive ability of the models.

The gene(s) identified as correlated with particular breast cancer outcomes relating to tamoxifen treatment by the above models provide the ability to focus gene expression analysis to only those genes that contribute to the ability to identify a subject as likely to have a particular outcome relative to another. The expression of other genes in a breast cancer cell would be relatively unable to provide information concerning, and thus assist in the discrimination of, a breast cancer outcome.

As will be appreciated by those skilled in the art, the models are highly useful with even a small set of reference gene expression data and can become increasingly accurate with the inclusion of more reference data although the incremental increase in accuracy will likely diminish with each additional datum. The preparation of additional reference gene expression data using genes identified and disclosed herein for discriminating between different outcomes in breast cancer following treatment with tamoxifen or another “antiestrogen” agent against breast cancer is routine and may be readily performed by the skilled artisan to permit the generation of models as described above to predict the status of an unknown sample based upon the expression levels of those genes.

To determine the (increased or decreased) expression levels of genes in the practice of the present invention, any method known in the art may be utilized. In one preferred embodiment of the invention, expression based on detection of RNA which hybridizes to the genes identified and disclosed herein is used. This is readily performed by any RNA detection or amplification+detection method known or recognized as equivalent in the art such as, but not limited to, reverse transcription-PCR, the methods disclosed in U.S. Pat. No. 6,794,141, and methods to detect the presence, or absence, of RNA stabilizing or destabilizing sequences.

Alternatively, expression based on detection of DNA status may be used. Detection of the DNA of an identified gene as methylated or deleted may be used for genes that have decreased expression in correlation with a particular breast cancer outcome. This may be readily performed by PCR based methods known in the art, including, but not limited to, Q-PCR. Conversely, detection of the DNA of an identified gene as amplified may be used for genes that have increased expression in correlation with a particular breast cancer outcome. This may be readily performed by PCR based, fluorescent in situ hybridization (FISH) and chromosome in situ hybridization (CISH) methods known in the art.

Expression based on detection of a presence, increase, or decrease in protein levels or activity may also be used. Detection may be performed by any immunohistochemistry (IHC) based, blood based (especially for secreted proteins), antibody (including autoantibodies against the protein) based, exfoliate cell (from the cancer) based, mass spectroscopy based, and image (including used of labeled ligand) based method known in the art and recognized as appropriate for the detection of the protein. Antibody and image based methods are additionally useful for the localization of tumors after determination of cancer by use of cells obtained by a non-invasive procedure (such as ductal lavage or fine needle aspiration), where the source of the cancerous cells is not known. A labeled antibody or ligand may be used to localize the carcinoma(s) within a patient or to assist in the enrichment of exfoliated cancer cells from a bodily fluid.

A preferred embodiment using a nucleic acid based assay to determine expression is by immobilization of one or more sequences of the genes identified herein on a solid support, including, but not limited to, a solid substrate as an array or to beads or bead based technology as known in the art. Alternatively, solution based expression assays known in the art may also be used. The immobilized gene(s) may be in the form of polynucleotides that are unique or otherwise specific to the gene(s) such that the polynucleotide would be capable of hybridizing to a DNA or RNA corresponding to the gene(s). These polynucleotides may be the full length of the gene(s) or be short sequences of the genes (up to one nucleotide shorter than the full length sequence known in the art by deletion from the 5′ or 3′ end of the sequence) that are optionally minimally interrupted (such as by mismatches or inserted non-complementary basepairs) such that hybridization with a DNA or RNA corresponding to the gene(s) is not affected. Preferably, the polynucleotides used are from the 3′ end of the gene, such as within about 350, about 300, about 250, about 200, about 150, about 100, or about 50 nucleotides from the polyadenylation signal or polyadenylation site of a gene or expressed sequence. Polynucleotides containing mutations relative to the sequences of the disclosed genes may also be used so long as the presence of the mutations still allows hybridization to produce a detectable signal.

The immobilized gene(s), or sequences complementary thereto, may be used to determine the state of nucleic acid samples prepared from sample breast cell(s) for which the outcome of the sample's subject (e.g. patient from whom the sample is obtained) is not known or for confirmation of an outcome that is already assigned to the sample's subject. Without limiting the invention, such a cell may be from a patient with ER+ or ER− breast cancer. The immobilized polynucleotide(s) need only be sufficient to specifically hybridize to the corresponding nucleic acid molecules derived from the sample under suitable conditions. While even a single correlated gene sequence may to able to provide adequate accuracy in discriminating between two breast cancer outcomes, two or more, three or more, four or more, five or more, six or more, seven or more, eight or more, nine or more, ten or more, eleven or more, or any integer number of the genes identified herein may be used as a subset capable of discriminating may be used in combination to increase the accuracy of the method. The invention specifically contemplates the selection of more than one, two or more, three or more, four or more, five or more, six or more, seven or more, eight or more, nine or more, ten or more, eleven or more, or any integer number of the genes disclosed in the tables and figures herein for use as a subset in the identification of breast cancer survival outcome.

Of course 1 or more, 2 or more, 3 or more, 4 or more, 5 or more, 6 or more, 7 or more, 8 or more, 9 or more, any integer number of, or all the genes provided in Tables 2, 3 and/or XXX below may be used. “Accession” as used in the context of the Tables herein as well as the present invention refers to the GenBank accession number of a sequence of each gene, the sequences of which are hereby incorporated by reference in their entireties as they are available from GenBank as accessed on the filing date of the present application. P value refers to values assigned as described in the Examples below. The indications of “E-xx” where “xx” is a two digit number refers to alternative notation for exponential figures where “E-xx” is “10^{-xx}”. Thus in combination with the numbers to the left of “E-xx”, the value being represented is the numbers to the left times 10^{-xx}. “Description” as used in the Tables provides a brief identifier of what the sequence/gene encodes.

Genes with a correlation identified by a p value below or about 0.02, below or about 0.01, below or about 0.005, or below or about 0.001 are preferred for use in the practice of the invention. The present invention includes the use of gene(s) the expression of which identify different breast cancer outcomes after treatment with TAM or another “antiestrogen” agent against breast cancer to permit simultaneous identification of breast cancer survival outcome of a patient based upon assaying a breast cancer sample from said patient.

In a second aspect, which also serves as embodiments of the use of a subset of the genes disclosed herein, the present invention relates to the identification and use of multiple sets of sequences for the determination of responsiveness of ER+ breast cancer to treatment with TAM or another “antiestrogen” agent against breast cancer. The differential expression of these sequences in breast cancer relative to normal breast cells is used to predict responsiveness to TAM or another “antiestrogen” agent against breast cancer in a subject.

To identify gene expression patterns in ER positive, early stage invasive breast cancers that might predict response to hormonal therapy, microarray gene expression analysis was performed on tumors from 60 women uniformly treated with adjuvant tamoxifen alone. These patients were identified from a total of 103 ER+ early stage cases presenting to Massachusetts General Hospital between 1987 and 1997, from whom tumor specimens were snap frozen and for whom minimal 5 year follow-up was available (see Table 1 for details). Within this cohort, 28 (46%) women developed distant metastasis with a median time to recurrence of 4 years (“tamoxifen non-responders”) and 32 (54%) women remained disease-free with median follow-up of 10 years (“tamoxifen responders”). Responders were matched with non-responder cases with respect to TNM staging (see Singletary, S. E. et al. “Revision of the American Joint Committee on Cancer staging system for breast cancer.” J Clin Oncol 20, 3628-36 (2002)) and tumor grade (see Dalton, L. W. et al. “Histologic grading of breast cancer: linkage of patient outcome with level of pathologist agreement.” Mod Pathol 13, 730-5. (2000)).

Previous studies linking gene expression profiles to clinical outcome in breast cancer have demonstrated that the potential for distant metastasis and overall survival probability may be predictable through biological characteristics of the primary tumor at the time of diagnosis (see Huang, E. et al. “Gene expression predictors of breast cancer outcomes.” Lancet 361, 1590-6 (2003); Sorlie, T. et al. “Gene expression patterns of breast carcinomas distinguish tumor subclasses with clinical implications.” Proc Natl Acad Sci USA 98:10869-74 (2001); Sorlie, T. et al. “Repeated observation of breast tumor subtypes in independent gene expression data sets.” Proc Natl Acad Sci USA 100, 8418-23 (2003); Sotiriou, C. et al. “Breast cancer classification and prognosis based on gene expression profiles from a population-based study.” Proc Natl Acad Sci USA 100, 10393-8 (2003); van 't Veer, L. J. et al. “Gene expression profiling predicts clinical outcome of breast cancer.” Nature 415, 530-6 (2002); and van de Vijver, M. J. et al. “A gene-expression signature as a predictor of survival in breast cancer.” N Engl J Med 347, 1999-2009 (2002)). In particular, a 70-gene expression signature has proven to be a strong prognostic factor, out-performing all known clinicopathological parameters. However, in those studies patients either received no adjuvant therapy (van 't Veer, L. J. et al. Nature 2002) or were treated non-uniformly with hormonal and chemotherapeutic regimens (Huang, E. et al.; Sorlie, T. et al.; Sorlie, T. et al.; Sotiriou, C. et al.; and van de Vijver, M. J. et al. N Engl J Med 2002). Patients with ER+ early-stage breast cancer treated with tamoxifen alone, such as the cohort studied here, represent only a subset of the population tested with the 70-gene signature. Of note, 61 of the genes in the 70-gene signature were present on the microarray used as described below, but no significant association with clinical outcome was observed in the defined subset of patients.

In comparison with existing biomarkers, including ESR1, PGR, ERBB2 and EGFR, the sets of gene sequences disclosed herein are significantly more predictive of responsiveness to TAM treatment. Multivariate analysis indicated that these three genes were significant predictors of clinical outcome independent of tumor size, nodal status and tumor grade. ER and progesterone receptor (PR) expression have been the major clinicopathological predictors for response to TAM. However, up to 40% of ER+ tumors fail to respond or develop resistance to TAM. The invention thus provides for the use of the identified biomarkers to allow better patient management by identifying patients who are more likely to benefit from TAM or other endocrine therapy and those who are likely to develop resistance and tumor recurrence.

As noted herein, the sequences(s) identified by the present invention are expressed in correlation with ER+ breast cancer cells. For example, IL17BR, identified by I.M.A.G.E. Consortium Clusters NM_{—}018725 and NM_{—}172234 (“The I.M.A.G.E. Consortium: An Integrated Molecular Analysis of Genomes and their Expression,” Lennon et al., 1996, Genomics 33:151-152; see also image.llnl.gov) has been found to be useful in predicting responsiveness to TAM treatment.

In preferred embodiments of the invention, any sequence, or unique portion thereof, of the IL17BR sequences of the cluster, as well as the UniGene Homo sapiens cluster Hs.5470, may be used. Similarly, any sequence encoding all or a part of the protein encoded by any IL17BR sequence disclosed herein may be used. Consensus sequences of I.M.A.G.E. Consortium clusters are as follows, with the assigned coding region (ending with a termination codon) underlined and preceded by the 5′ untranslated and/or non-coding region and followed by the 3′ untranslated and/or non-coding region:

SEQ ID NO:1 (consensus sequence for IL17BR, transcript variant 1, identified as NM_{—}018725 or NM_{—}018725.2):

agcgcagcgt gcgggtggcc tggatcccgc gcagtggccc ggcgatgtcg ctcgtgctgc
taagcctggc cgcgctgtgc aggagcgccg taccccgaga gccgaccgtt caatgtggct
ctgaaactgg gccatctcca gagtggatgc tacaacatga tctaatcccc ggagacttga
gggacctccg agtagaacct gttacaacta gtgttgcaac aggggactat tcaattttga
tgaatgtaag ctgggtactc cgggcagatg ccagcatccg cttgttgaag gccaccaaga
tttgtgtgac gggcaaaagc aacttccagt cctacagctg tgtgaggtgc aattacacag
aggccttcca gactcagacc agaccctctg gtggtaaatg gacattttcc tacatcggct
tccctgtaga gctgaacaca gtctatttca ttggggccca taatattcct aatgcaaata
tgaatgaaga tggcccttcc atgtctgtga atttcacctc accaggctgc ctagaccaca
taatgaaata taaaaaaaag tgtgtcaagg ccggaagcct gtgggatccg aacatcactg
cttgtaagaa gaatgaggag acagtagaag tgaacttcac aaccactccc ctgggaaaca
gatacatggc tcttatccaa cacagcacta tcatcgggtt ttctcaggtg tttgagccac
accagaagaa acaaacgcga gcttcagtgg tgattccagt gactggggat agtgaaggtg
ctacggtgca gctgactcca tattttccta cttgtggcag cgactgcatc cgacataaag
gaacagttgt gctctgccca caaacaggcg tccctttccc tctggataac aacaaaagca
agccgggagg ctggctgcct ctcctcctgc tgtctctgct ggtggccaca tgggtgctgg
tggcagggat ctatctaatg tggaggcacg aaaggatcaa gaagacttcc ttttctacca
ccacactact gccccccatt aaggttcttg tggtttaccc atctgaaata tgtttccatc
acacaatttg ttacttcact gaatttcttc aaaaccattg cagaagtgag gtcatccttg
aaaagtggca gaaaaagaaa atagcagaga tgggtccagt gcagtggctt gccactcaaa
agaaggcagc agacaaagtc gtcttccttc tttccaatga cgtcaacagt gtgtgcgatg
gtacctgtgg caagagcgag ggcagtccca gtgagaactc tcaagacctc ttcccccttg
cctttaacct tttctgcagt gatctaagaa gccagattca tctgcacaaa tacgtggtgg
tctactttag agagattgat acaaaagacg attacaatgc tctcagtgtc tgccccaagt
accacctcat gaaggatgcc actgctttct gtgcagaact tctccatgtc aagcagcagg
tgtcagcagg aaaaagatca caagcctgcc acgatggctg ctgctccttg tagcccaccc
atgagaagca agagacctta aaggcttcct atcccaccaa ttacagggaa aaaacgtgtg
atgatcctga agcttactat gcagcctaca aacagcctta gtaattaaaa cattttatac
caataaaatt ttcaaatatt gctaactaat gtagcattaa ctaacgattg gaaactacat
ttacaacttc aaagctgttt tatacataga aatcaattac agttttaatt gaaaactata
accattttga taatgcaaca ataaagcatc ttcagccaaa catctagtct tccatagacc
atgcattgca gtgtacccag aactgtttag ctaatattct atgtttaatt aatgaatact
aactctaaga acccctcact gattcactca atagcatctt aagtgaaaaa ccttctatta
catgcaaaaa atcattgttt ttaagataac aaaagtaggg aataaacaag ctgaacccac
ttttaaaaaa aaaaaaaaaa aaaaaaaaaa aaaaaaa

SEQ ID NO:2 (consensus sequence for IL17BR, transcript variant 2, identified as NM_{—}172234 or NM_{—}172234.1):

agcgcagcgt gcgggtggcc tggatcccgc gcagtggccc ggcgatgtcg ctcgtgctgc
taagcctggc cgcgctgtgc aggagcgccg taccccgaga gccgaccgtt caatgtggct
ctgaaactgg gccatctcca gagtggatgc tacaacatga tctaatcccc ggagacttga
gggacctccg agtagaacct gttacaacta gtgttgcaac aggggactat tcaattttga
tgaatgtaag ctgggtactc cgggcagatg ccagcatccg cttgttgaag gccaccaaga
tttgtgtgac gggcaaaagc aacttccagt cctacagctg tgtgaggtgc aattacacag
aggccttcca gactcagacc agaccctctg gtggtaaatg gacattttcc tacatcggct
tccctgtaga gctgaacaca gtctatttca ttggggccca taatattcct aatgcaaata
tgaatgaaga tggcccttcc atgtctgtga atttcacctc accaggctgc ctagaccaca
taatgaaata taaaaaaaag tgtgtcaagg ccggaagcct gtgggatccg aacatcactg
cttgtaagaa gaatgaggag acagtagaag tgaacttcac aaccactccc ctgggaaaca
gatacatggc tcttatccaa cacagcacta tcatcgggtt ttctcaggtg tttgagccac
accagaagaa acaaacgcga gcttcagtgg tgattccagt gactggggat agtgaaggtg
ctacggtgca ggtaaagttc agtgagctgc tctggggagg gaagggacat agaagactgt
tccatcattc attgctttta aggatgagtt ctctcttgtc aaatgcactt ctgccagcag
acaccagtta agtggcgttc atgggggctc tttcgctgca gcctccaccg tgctgaggtc
aggaggccga cgtggcagtt gtggtccctt ttgcttgtat taatggctgc tgaccttcca
aagcactttt tattttcatt ttctgtcaca gacactcagg gatagcagta ccattttact
tccgcaagcc tttaactgca agatgaagct gcaaagggtt tgaaatggga aggtttgagt
tccaggcagc gtatgaactc tggagagggg ctgccagtcc tctctgggcc gcagcggacc
cagctggaac acaggaagtt ggagcagtag gtgctccttc acctctcagt atgtctcttt
caactctagt ttttgaggtg gggacacagg aggtccagtg ggacacagcc actccccaaa
gagtaaggag cttccatgct tcattccctg gcataaaaag tgctcaaaca caccagaggg
ggcaggcacc agccagggta tgatggctac tacccttttc tggagaacca tagacttccc
ttactacagg gacttgcatg tcctaaagca ctggctgaag gaagccaaga ggatcactgc
tgctcctttt ttctagagga aatgtttgtc tacgtggtaa gatatgacct agccctttta
ggtaagcgaa ctggtatgtt agtaacgtgt acaaagttta ggttcagacc ccgggagtct
tgggcacgtg ggtctcgggt cactggtttt gactttaggg ctttgttaca gatgtgtgac
caaggggaaa atgtgcatga caacactaga ggtatgggcg aagccagaaa gaagggaagt
tttggctgaa gtaggagtct tggtgagatt ttgctctgat gcatggtgtg aactttctga
gcctcttgtt tttcctcagc tgactccata ttttcctact tgtggcagcg actgcatccg
acataaagga acagttgtgc tctgcccaca aacaggcgtc cctttccctc tggataacaa
caaaagcaag ccgggaggct ggctgcctct cctcctgctg tctctgctgg tggccacatg
ggtgctggtg gcagggatct atctaatgtg gaggcacgaa aggatcaaga agacttcctt
ttctaccacc acactactgc cccccattaa ggttcttgtg gtttacccat ctgaaatatg
tttccatcac acaatttgtt acttcactga atttcttcaa aaccattgca gaagtgaggt
catccttgaa aagtggcaga aaaagaaaat agcagagatg ggtccagtgc agtggcttgc
cactcaaaag aaggcagcag acaaagtcgt cttccttctt tccaatgacg tcaacagtgt
gtgcgatggt acctgtggca agagcgaggg cagtcccagt gagaactctc aagacctctt
cccccttgcc tttaaccttt tctgcagtga tctaagaagc cagattcatc tgcacaaata
cgtggtggtc tactttagag agattgatac aaaagacgat tacaatgctc tcagtgtctg
ccccaagtac cacctcatga aggatgccac tgctttctgt gcagaacttc tccatgtcaa
gcagcaggtg tcagcaggaa aaagatcaca agcctgccac gatggctgct gctccttgta
gcccacccat gagaagcaag agaccttaaa ggcttcctat cccaccaatt acagggaaaa
aacgtgtgat gatcctgaag cttactatgc agcctacaaa cagccttagt aattaaaaca
ttttatacca ataaaatttt caaatattgc taactaatgt agcattaact aacgattgga
aactacattt acaacttcaa agctgtttta tacatagaaa tcaattacag ttttaattga
aaactataac cattttgata atgcaacaat aaagcatctt cagccaaaca tctagtcttc
catagaccat gcattgcagt gtacccagaa ctgtttagct aatattctat gtttaattaa
tgaatactaa ctctaagaac ccctcactga ttcactcaat agcatcttaa gtgaaaaacc
ttctattaca tgcaaaaaat cattgttttt aagataacaa aagtagggaa taaacaagct
gaacccactt ttaaaaaaaa aaaaaaaaaa aaaaaaaaaa aaaaa

I.M.A.G.E. Consortium Clone ID numbers and the corresponding GenBank accession numbers of sequences identified as belonging to the I.M.A.G.E. Consortium and UniGene clusters, are listed below. Also included are sequences that are not identified as having a Clone ID number but still identified as being those of IL17BR. The sequences include those of the “sense” and complementary strands sequences corresponding to IL17BR. The sequence of each GenBank accession number is presented in the attached Appendix to PCT/US04/30789.

| Clone ID | |
|---|---|
| numbers | GenBank accession numbers |
| 2985728 | AW675096, AW673932, BC000980 |
| 5286745 | BI602183 |
| 5278067 | BI458542 |
| 5182255 | BI823321 |
| 924000 | AA514396 |
| 3566736 | BF110326 |
| 3195409 | BE466508 |
| 3576775 | BF740045 |
| 2772915 | AW299271 |
| 1368826 | AA836217 |
| 1744837 | AI203628 |
| 2285564 | AI627783 |
| 2217709 | AI744263 |
| 2103651 | AI401622 |
| 2419487 | AI826949 |
| 3125592 | BE047352 |
| 2284721 | AI911549 |
| 3643302 | BF194822 |
| 1646910 | AI034244 |
| 1647001 | AI033911 |
| 3323709 | BF064177 |
| 1419779 | AA847767 |
| 2205190 | AI538624 |
| 2295838 | AI913613 |
| 2461335 | AI942234 |
| 2130362 | AI580483 |
| 2385555 | AI831909 |
| 2283817 | AI672344 |
| 2525596 | AW025192 |
| 454687 | AA677205 |
| 1285273 | AA721647 |
| 3134106 | BF115018 |
| 342259 | W61238, W61239 |
| 1651991 | AI032064 |
| 2687714 | AW236941 |
| 3302808 | BG057174 |
| 2544461 | AW058532 |
| 122014 | T98360, T98361 |
| 2139250 | AI470845 |
| 2133899 | AI497731 |
| 121300 | T96629, T96740 |
| 162274 | H25975, H25941 |
| 3446667 | BE539514, BX282554 |
| 156864 | R74038, R74129 |
| 4611491 | BG433769 |
| 4697316 | BG530489 |
| 429376 | AA007528, AA007529 |
| 5112415 | BI260259 |
| 701357 | AA287951, AA287911 |
| 121909 | T97852, T97745 |
| 268037 | N40294 |
| 1307489 | AA809841 |
| 1357543 | AA832389 |
| 48442 | H14692 |
| 1302619 | AA732635 |
| 1562857 | AA928257 |
| 1731938 | AI184427 |
| 1896025 | AI298577 |
| 2336350 | AI692717 |
| 1520997 | AA910922 |
| 240506 | H90761 |
| 2258560 | AI620122 |
| 1569921 | AI793318, AA962325, AI733290 |
| 6064627 | BQ226353 |
| 299018 | W04890 |
| 5500181 | BM455231 |
| 2484011 | BI492426 |
| 4746376 | BG674622 |
| 233783 | BX111256 |
| 1569921 | BX117618 |
| 450450 | AA682806 |
| 1943085 | AI202376 |
| 2250390 | AI658949 |
| 4526156 | BG403405 |
| 3249181 | BE673417 |
| 2484395 | AW021469 |
| 30515867 | CF455736 |
| 2878155 | AW339874 |
| 4556884 | BG399724 |
| 3254505 | BF475787 |
| 3650593 | BF437145 |
| 233783 | H64601 |
| None (mRNA | AF212365, AF208110, AF208111, AF250309, |
| sequences) | AK095091 |
| None | BM983744, CB305764, BM715988, BM670929, |
| | BI792416, BI715216, N56060, CB241389, |
| | AV660618, BX088671, CB154426, CA434589, |
| | CA412162, CA314073, BF921554, BF920093, |
| | AV685699, AV650175, BX483104, CD675121, |
| | BE081436, AW970151, AW837146, AW368264, |
| | D25960, AV709899, BX431018, AL535617, |
| | AL525465, BX453536, BX453537, AV728945, |
| | AV728939, AV727345 |

In one preferred embodiment, any sequence, or unique portion thereof, of the following IL17BR sequence, identified by AF208111 or AF208111.1, may be used in the practice of the invention.

SEQ ID NO:3 (sequence for IL17BR):

CGGCGATGTCGCTCGTGCTGATAAGCCTGGCCGCGCTGTGCAGGAGCGCCGTACCCCGAG
AGCCGACCGTTCAATGTGGCTCTGAAACTGGGCCATCTCCAGAGTGGATGCTACAACATG
ATCTAATCCCCGGAGACTTGAGGGACCTCCGAGTAGAACCTGTTACAACTAGTGTTGCAA
CAGGGGACTATTCAATTTTGATGAATGTAAGCTGGGTACTCCGGGCAGATGCCAGCATCC
GCTTGTTGAAGGCCACCAAGATTTGTGTGACGGGCAAAAGCAACTTCCAGTCCTACAGCT
GTGTGAGGTGCAATTACACAGAGGCCTTCCAGACTCAGACCAGACCCTCTGGTGGTAAAT
GGACATTTTCCTATATCGGCTTCCCTGTAGAGCTGAACACAGTCTATTTCATTCGGGCCC
ATAATATTCCTAATGCAAATATGAATGAAGATGGCCCTTCCATGTCTGTGAATTTCACCT
CACCAGGCTGCCTAGACCACATAATGAAATATAAAPAAAAGTGTGTCAAGGCCGGAAGCC
TGTGGGATCCGAACATCACTGCTTGTAAGAAGAATGAGGAGACAGTAGAAGTGAACTTCA
CAACCACTCCCCTGGGAAACAGATACATGGCTCTTATCCAACACAGCACTATCATCGGGT
TTTCTCAGGTGTTTGAGCCACACCAGAAGAAACAAACGCGAGCTTCAGTGGTGATTCCAG
TGACTGGGGATAGTGAAGGTGCTACGGTGCAGGTAAAGTTCAGTGAGCTGCTCTGGGGAG
GGAAGGGACATAGAAGACTGTTCCATCATTCATTGCTTTTAAGGATGAGTTCTCTCTTGT
CAAATGCACTTCTGCCAGCAGACACCAGTTAAGTGGCGTTCATGGGGGTTCTTTCGCTGC
AGCCTCCACCGTGCTGAGGTCAGGAGGCCGACGTGGCAGTTGTGGTCCCTTTTGCTTGTA
TTAATGGCTGCTGACCTTCCAAAGCACTTTTTATTTTCATTTTCTGTCACAGACACTCAG
GGATAGCAGTACCATTTTACTTCCGCAAGCCTTTAACTGCAAGATGAAGCTGCAAAGGGT
TTGAAATGGGAAGGTTTGAGTTCCAGGCAGCGTATGAACTCTGGAGAGGGGCTGCCAGTC
CTCTCTGGGCCGCAGCGGACCCAGCTGGAACACAGGAAGTTGGAGCAGTAGGTGCTCCTT
CACCTCTCAGTATGTCTCTTTCAACTCTAGTTTTTGAAGTGGGGACACAGGAAGTCCAGT
GGGGACACAGCCACTCCCCAAAGAATAAGGAACTTCCATGCTTCATTCCCTGGCATAAAA
AGTGNTCAAACACACCAGAGGGGGCAGGCACCAGCCAGGGTATGATGGGTACTACCCTTT
TCTGGAGAACCATAGACTTCCCTTACTACAGGGACTTGCATGTCCTAAAGCACTGGCTGA
AGGAAGCCAAGAGGATCACTGCTGCTCCTTTTTTGTAGAGGAAATGTTTGTGTACGTGGT
AAGATATGACCTAGCCCTTTTAGGTAAGCGAACTGGTATGTTAGTAACGTGTACAAAGTT
TAGGTTCAGACCCCGGGAGTCTTGGGCATGTGGGTCTCGGGTCACTGGTTTTGACTTTAG
GGCTTTGTTACAGATGTGTGACCAAGGGGAAAATGTGCATGACAACACTAGAGGTAGGGG
CGAAGCCAGAAAGAAGGGAAGTTTTGGCTGAAGTAGGAGTCTTGGTGAGATTTTGCTGTG
ATGCATGGTGTGAACTTTCTGAGCCTCTTGTTTTTCCTCAGCTGACTCCATATTTTCCTA
CTTGTGGCAGCGACTGCATCCGACATAAAGGAACAGTTGTGCTCTGCCCACAAACAGGCG
TCCCTTTCCCTCTGGATAACAACAAAAGCAAGCCGGGAGGCTGGCTGCCTCTCCTCCTGC
TGTCTCTGCTGGTGGCCACATGGGTGCTGGTGGCAGGGATCTATCTAATGTGGAGGCACG
AAAGGATCAAGAAGACTTCCTTTTCTACCACCACACTACTGCCCCCCATTAAGGTTCTTG
TGGTTTACCCATCTGAAATATGTTTCCATCACACAATTTGTTACTTCACTGAATTTCTTC
AAAACCATTGCAGAAGTGAGGTCATCCTTGAAAAGTGGCAGAAAAAGAAAATAGCAGAGA
TGGGTCCAGTGCAGTGGCTTGCCACTCAAAAGAAGGCAGCAGACAAAGTCGTCTTCCTTC
TTTCCAATGACGTCAACAGTGTGTGCGATGGTACCTGTGGCAAGAGCGAGGGCAGTCCCA
GTGAGAACTCTCAAGACCTCTTCCCCCTTGCCTTTAACCTTTTCTGCAGTGATCTAAGAA
GCCAGATTCATCTGCACAAATACGTGGTGGTCTACTTTAGAGAGATTGATACAAAAGACG
ATTACAATGCTCTCAGTGTCTGCCCCAAGTACCACTTCATGAAGGATGCCACTGCTTTCT
GTGCAGAACTTCTCCATGTCAAGCAGCAGGTGTCAGCAGGAAAAAGATCACAAGCCTGCC
ACGATGGCTGCTGCTCCTTGTAGCCCACCCATGAGAAGCAAGAGACCTTAAAGGCTTCCT
ATCCCACCAATTACAGGGAAAAAACGTGTGATGATCCTGAAGCTTACTATGCAGCCTACA
AACAGCCTTAGTAATTAAAACATTTTATACCAATAAAATTTTCAAATATTACTAACTAAT
GTAGCATTAACTAACGATTGGAAACTACATTTACAACTTCAAAGCTGTTTTATACATAGA
AATCAATTACAGCTTTAATTGAAAACTGTAACCATTTTGATAATGCAACAATAAAGCATC
TTCCAAAAAAAAAAAAAAAAAAAAAAAAAAAAAAAA

In preferred embodiments of the invention, any sequence, or unique portion thereof, of the CHDH sequences of the cluster, as well as the UniGene Homo sapiens cluster Hs. 126688, may be used. Similarly, any sequence encoding all or a part of the protein encoded by any CHDH sequence disclosed herein, including sequences of the new assembled contig, may be used. Consensus sequences of I.M.A.G.E. Consortium clusters are as follows, with the assigned coding region (ending with a termination codon) underlined and preceded by the 5′ untranslated and/or non-coding region and followed by the previously identified 3′ untranslated and/or non-coding region:

SEQ ID NO:4 (consensus sequence for CHDH, identified as NM_{—}018397 or NM_{—}018397.1):

agcgggccgc ggccacccgc tcctcccgct ccggtcccga
ctgtcgggct ctcggccgag
tcgccccgga caatcacaaa gagtgtgtag gccagccccg
gtcacagagt gcaccgtatc
ctgtcacttc tggatgtgag ggagaagtga gtcatctcat
tcccctccgt ggatcagagg
acttggacta gatagaagca tgtggtgtct cctacgaggc
ctgggccggc ctggagccct
ggcacgggga gccctggggc agcagcaatc cctgggtgcc
cgggccctgg ccagcgcagg
ctctgagagc cgggacgagt acagctatgt ggtggtgggc
gcgggctcgg cgggctgcgt
gctggctggg aggctcacgg aggaccccgc cgagcgcgtg
ctgctgctgg aggccgggcc
caaggacgtg cgcgcgggga gcaagcggct ctcgtggaag
atccacatgc ccgcggccct
ggtggccaac ctgtgcgacg acaggtacaa ctggtgctac
cacacagagg tgcagcgggg
cctggacggc cgcgtgctgt actggccacg cggccgcgtc
tggggtggct cctcatccct
caatgccatg gtctacgtcc gtgggcacgc cgaggactac
gagcgctggc agcgccaggg
cgcccgcggc tgggactacg cgcactgcct gccctacttc
cgcaaggcgc agggccacga
gctgggcgcc agccggtacc ggggcgccga tggcccgctg
cgggtgtccc ggggcaagac
caaccacccg ctgcactgcg cattcctgga ggccacgcag
caggccggct acccgctcac
cgaggacatg aatggcttcc agcaggaggg cttcggctgg
atggacatga ccatccatga
aggcaaacgg tggagcgcag cctgtgccta cctgcaccca
gcactgagcc gcaccaacct
caaggccgag gccgagacgc ttgtgagcag ggtgctattt
gagggcaccc gtgcagtggg
cgtggagtat gtcaagaatg gccagagcca cagggcttat
gccagcaagg aggtgattct
gagtggaggt gccatcaact ctccacagct gctcatgctc
tctggcatcg ggaatgctga
tgacctcaag aaactgggca tccctgtggt gtgccaccta
cctggggttg gccagaacct
gcaagaccac ctggagatct acattcagca ggcatgcacc
cgccctatca ccctccattc
agcacagaag cccctgcgga aggtctgcat tggtctggag
tggctctgga aattcacagg
ggagggagcc actgcccatc tggaaacagg tgggttcatc
cgcagccagc ctggggtccc
ccacccggac atccagttcc atttcctgcc atcccaagtg
attgaccacg ggcgggtccc
cacccagcag gaggcttacc aggtacatgt ggggcccatg
cggggcacga gtgtgggctg
gctcaaactg agaagtgcca atccccaaga ccaccctgtg
atccagccca actacttgtc
aacagaaact gatattgagg atttccgtct gtgtgtgaag
ctcaccagag aaatttttgc
acaggaagcc ctggctccgt tccgagggaa agagctccag
ccaggaagcc acattcagtc
agataaagag atagatgcct ttgtgcgggc aaaagccgac
agcgcctacc acccctcgtg
cacctgtaag atgggccagc cctccgatcc cactgccgtg
gtggatccgc agacaagggt
cctcggggtg gaaaacctca gggtcgtcga tgcctccatc
atgcctagca tggtcagcgg
caacctgaac gcccccacaa tcatgatcgc agagaaggca
gctgacatta tcaaggggca
gcctgcactc tgggacaaag atgtccctgt ctacaagccc
aggacgctgg ccacccagcg
ctaagacagt tgctgctgga ggatgaccag ggaagccccc
tgataagcca agagggccag
cacagccctt gctcccaggc tcctgcctga aactatctag
cacactagga cccaggtggt
accctactca gtggctgaga attggataaa gtcttgggaa
atgagacaaa aaaaaaaaaa
aa

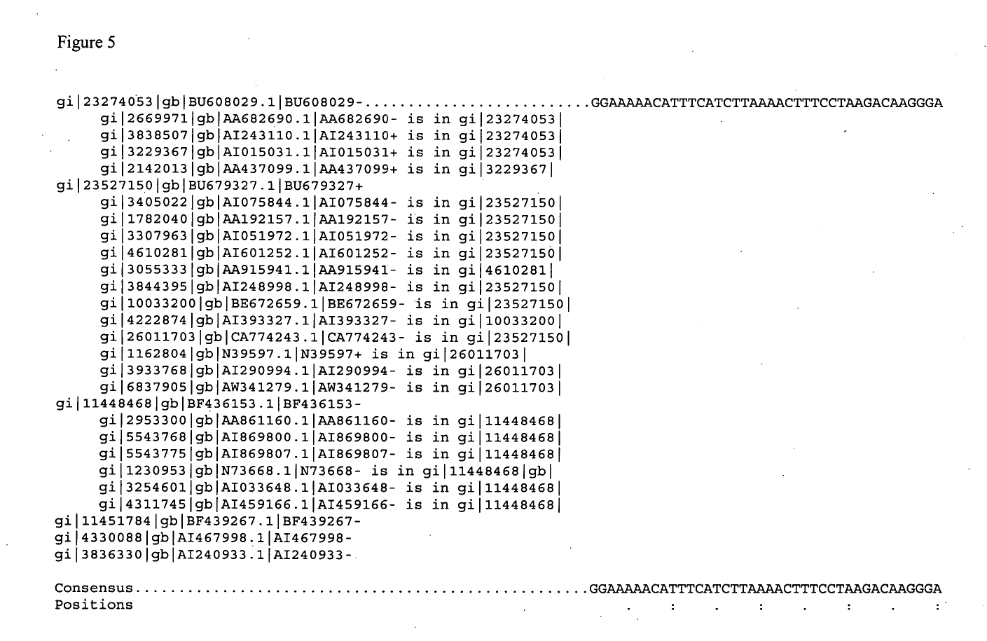

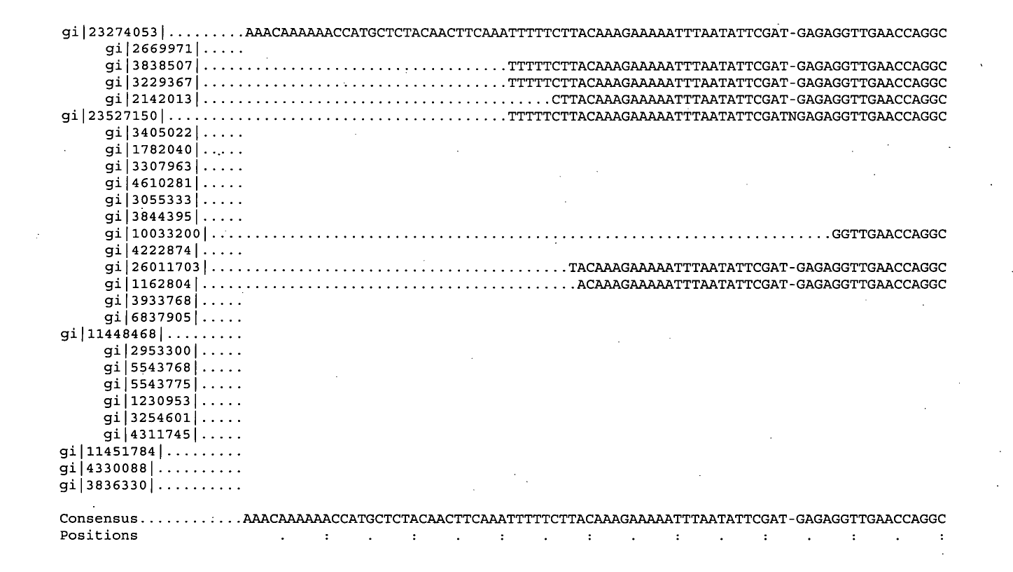

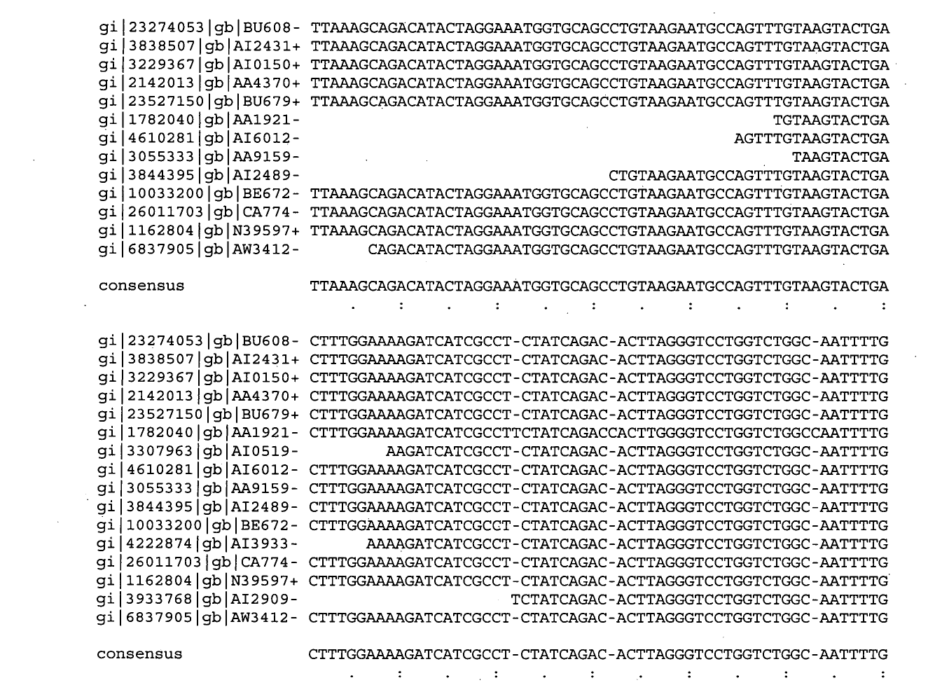

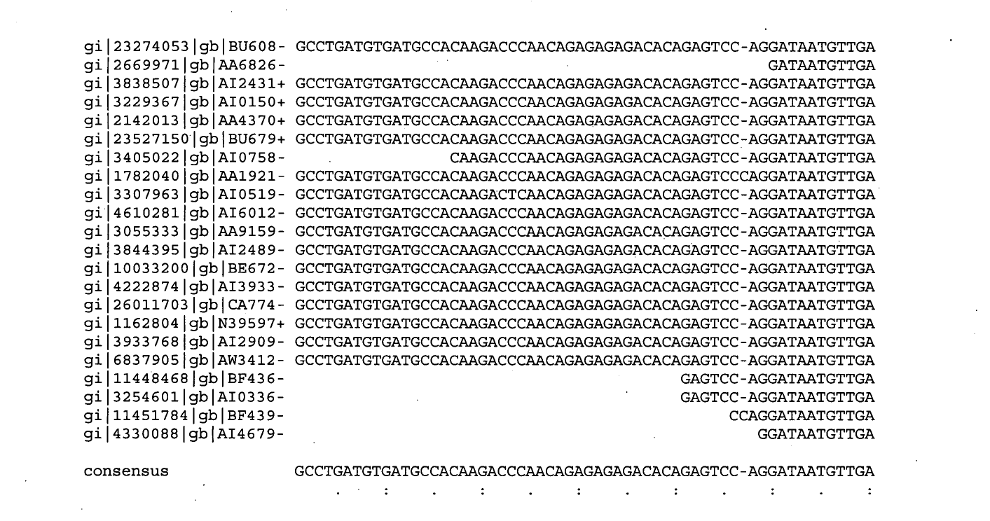

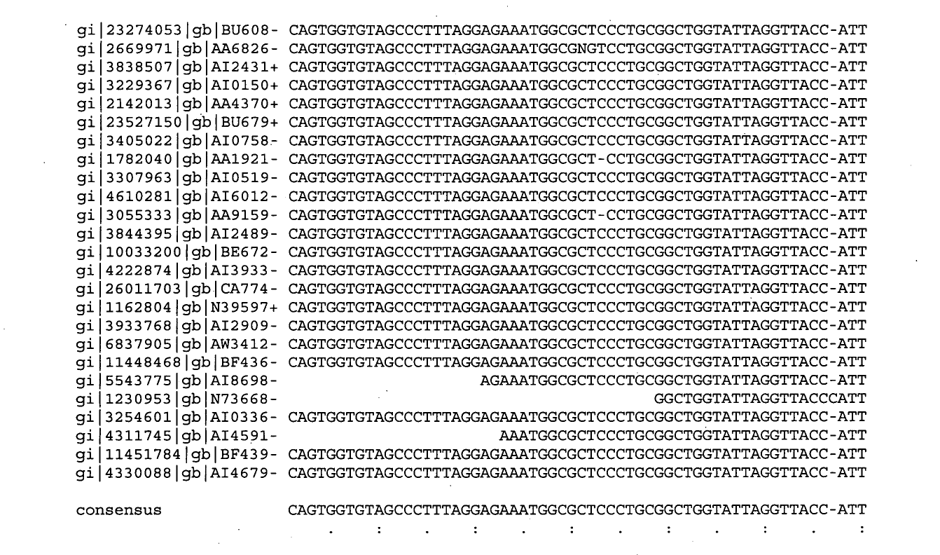

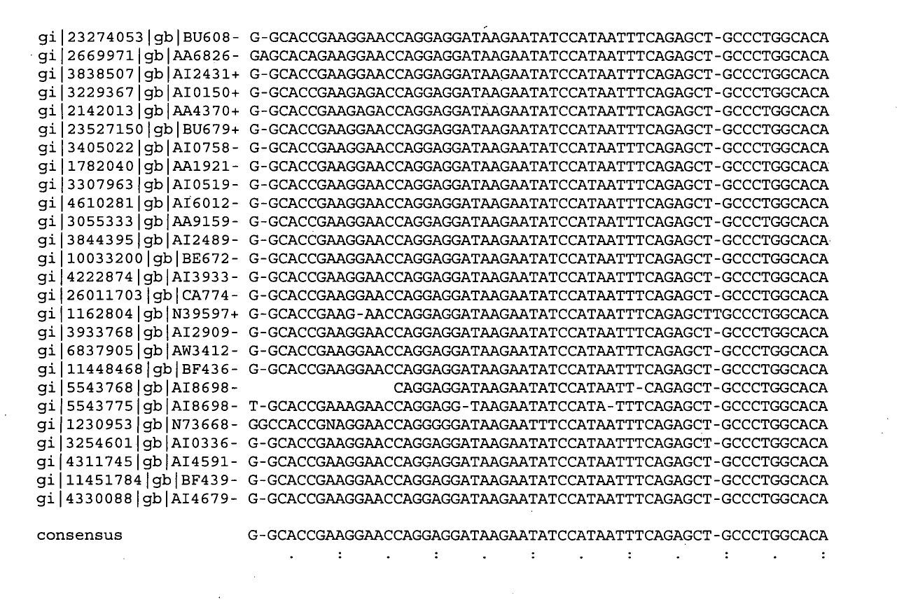

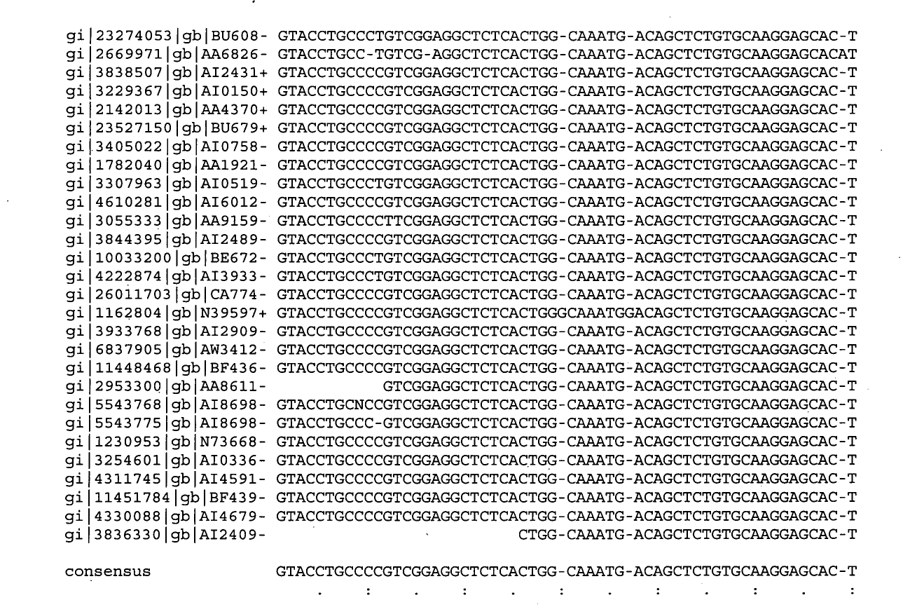

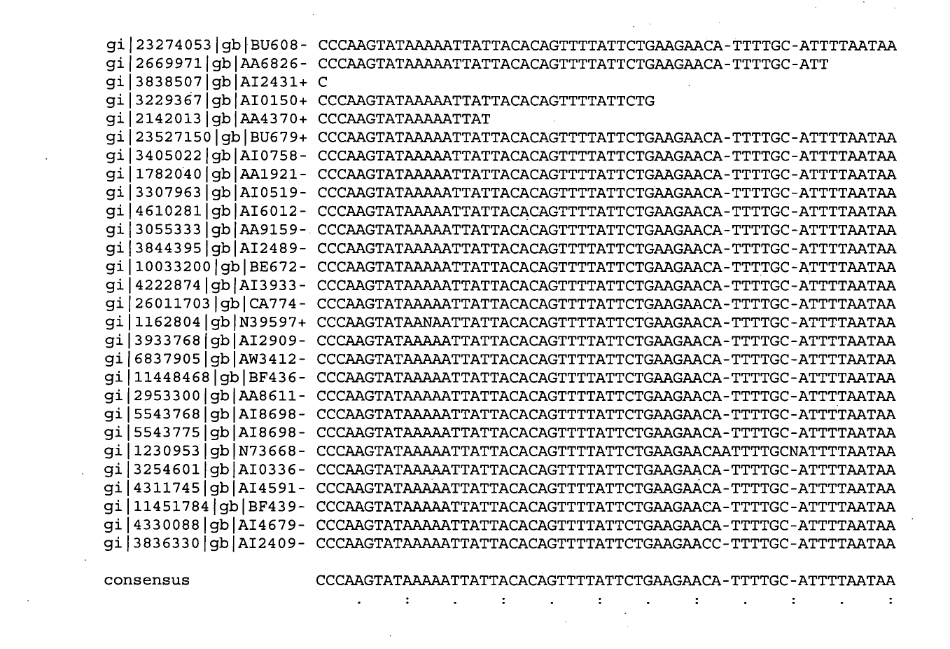

I.M.A.G.E. Consortium Clone ID numbers and the corresponding GenBank accession numbers of sequences identified as belonging to the I.M.A.G.E. Consortium and UniGene clusters, are listed below. Also included are sequences that are not identified as having a Clone ID number but still identified as being those of CHDH. The sequences include those of the “sense” and complementary strands sequences corresponding to CHDH. Additional sequences for use in the practice of the invention are those aligned in FIG. 5, which are also provided in the attached Appendix to PCT/US04/30789.

Clone ID number/GenBank accession number(s)

4824572/BC034502 and BG720228
5191415/BI765156
5311690/BI667529
5267676/BI460380
1031605/AA609488
3842653/BE732217
4543273/BG336766
3504516/BE279319
3140587/BE279968
6297066/BQ648069
2734263/AW449121
2735859/AW450678
2720363/AW139168
3642981/BF195860
5931105/BQ066460
3574335/BF430927
3268842/BF435866
3267752/BF435185
1868020/AI264647 and BX116752 and AI733810 and AI792632
2365837/AI741739
3085519/BF510364
1647746/AI034449
2695349/AW194822
2285283/AI628996
2694067/AW235087
2285315/AI629023
2463061/AI928186
2462306/AI927042
2381448/AI768443
2298488/AI650346
3134601/BF197300
2300327/AI631941
2697626/AW167538
3034918/AW779820
2525301/AW024823
2300291/AI631914
2137091/AI473735
4147169/BG060119
2772286/AW299654
2172535/AI564145
2690214/AW241612
1868068/AI241086
1608918/AA991365
3134810/BF197431
1869723/AI245204
2691133/AW242403
6109050/BU500214
2384051/AI796286
2055388/AI308167
3032446/AW771262
2907815/AW340332
1636795//AI792354 and AI017355
2299592/AI640195
2054920/AI334627
2690173/AW237735
1869819/AI245373
3195030/BE464406
1646613/AI025866
2773291/AW299629
2461358/AI942245
5678397/BM142449 and BM142311
5672209/BM052814 and BM053126
2137904/AI800207
511224/AA088689 and AA088826
2734357/AW449405
381379/AA052926 and AA052927
2337545/AI914219
2528186/AW337722
2028284/AI262965
3436048/BF940636
2344677/AI695649
123940/R00867 and R01524/
240988/H90906 and H91018
240077/H82409 and H82667
None (mRNA sequences)/NM_018397.1
and AJ272267.1 and AK055402.1
None/AA772473.1 and BM682615.1 and BM713059.1
and BM716959.1 and BU738538.1 and
AA324019.1 and AA302740.1 and C20981.1 and BF930030.1
and BQ303877.1 and BM769931.1 and
AW900269.1 and F26419.1 and CB147231.1 and
BE765491.1 AV656671.1

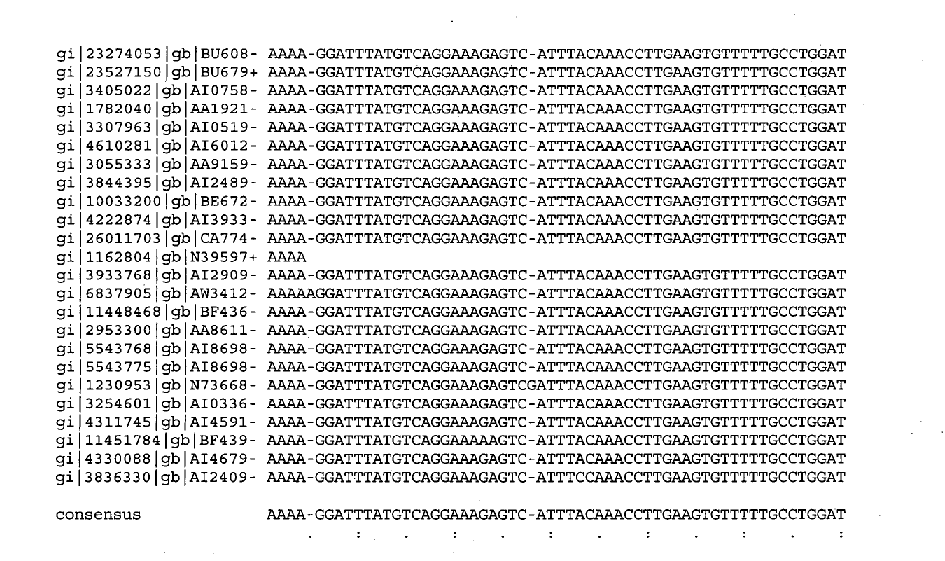

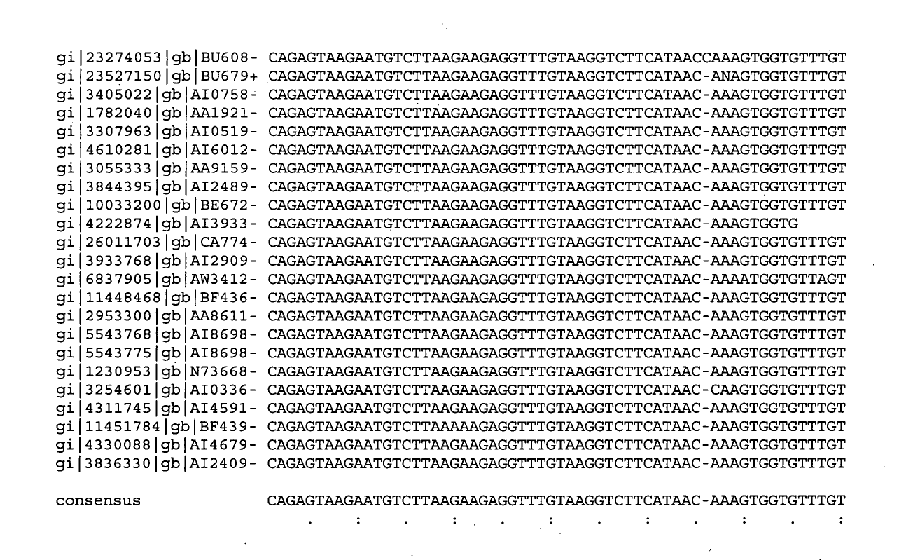

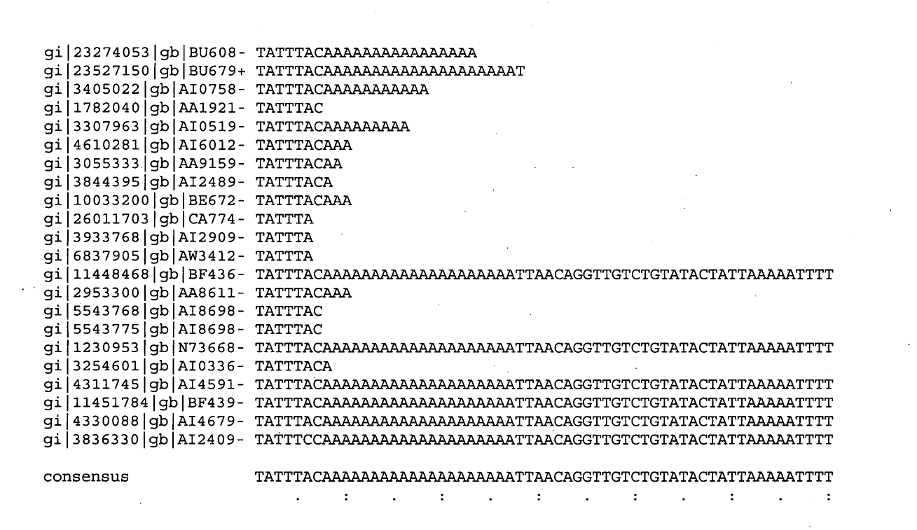

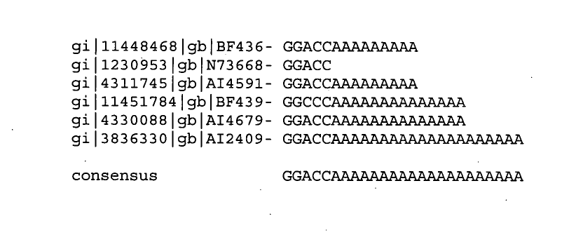

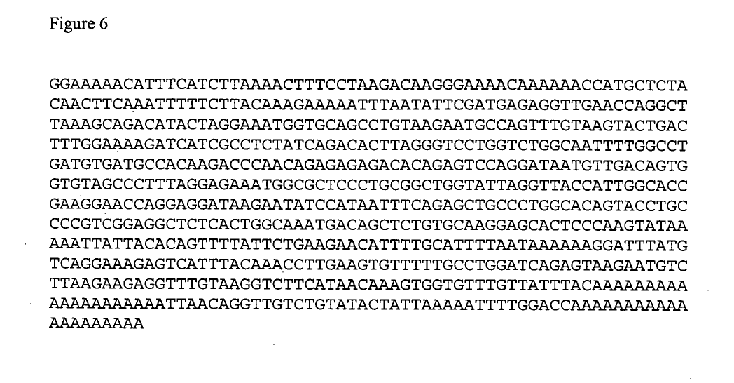

In one preferred embodiment, any sequence, or unique portion thereof, of the CHDH sequences in FIG. 5 or 6 may be used in the practice of the invention.

In another set of preferred embodiments of the invention, any sequence, or unique portion thereof, of the QPRT sequences of the I.M.A.G.E. Consortium cluster NM_{—}014298, as well as the UniGene Homo sapiens cluster Hs.126688, may be used. Similarly, any sequence encoding all or a part of the protein encoded by any QPRT sequence disclosed herein may be used. The consensus sequence of the I.M.A.G.E. Consortium cluster is as follows, with the assigned coding region (ending with a termination codon) underlined and preceded by the 5′ untranslated and/or non-coding region and followed by the 3′ untranslated and/or non-coding region:

SEQ ID NO:5 (consensus sequence for QPRT, identified as NM_{—}014298 or NM_{—}014298.2):

gtcctgagca gccaacacac cagcccagac agctgcaagt
caccatggac gctgaaggcc
tggcgctgct gctgccgccc gtcaccctgg cagccctggt
ggacagctgg ctccgagagg
actgcccagg gctcaactac gcagccttgg tcagcggggc
aggcccctcg caggcggcgc
tgtgggccaa atcccctggg gtactggcag ggcagccttt
cttcgatgcc atatttaccc
aactcaactg ccaagtctcc tggttcctcc ccgagggatc
gaagctggtg ccggtggcca
gagtggccga ggtccggggc cctgcccact gcctgctgct
gggggaacgg gtggccctca
acacgctggc ccgctgcagt ggcattgcca gtgctgccgc
cgctgcagtg gaggccgcca
ggggggccgg ctggactggg cacgtggcag gcacgaggaa
gaccacgcca ggcttccggc
tggtggagaa gtatgggctc ctggtgggcg gggccgcctc
gcaccgctac gacctgggag
ggctggtgat gttgaaggat aaccatgtgg tgccccccgg
tggcgtggag aaggcggtgc
gggcggccag acaggcggct gacttcgctc tgaaggtgga
agtggaatgc agcagcctgc
aggaggtcgt ccaggcagct gaggctggcg ccgaccttgt
cctgctggac aacttcaagc
cagaggagct gcaccccacg gccaccgcgc tgaaggccca
gttcccgagt gtggctgtgg
aagccagtgg gggcatcacc ctggacaacc tcccccagtt
ctgcgggccg cacatagacg
tcatctccat ggggatgctg acccaggcgg tcccagccct
tgatttctcc ctcaagctgt
ttgccaaaga ggtggctcca gtgcccaaaa tccactagtc
ctaaaccgga agaggatgac
accggccatg ggttaacgtg gctcctcagg accctctggg
tcacacatct ttagggtcag
tgaacaatgg ggcacatttg gcactagctt gagcccaact
ctggctctgc cacctgctgc
tcctgtgacc tgtcagggct gacttcacct ctgctcatct
cagtttccta atctgtaaaa
tgggtctaat aaaggatcaa ccaaaaaaaa aaaaaaaaaa
aa

I.M.A.G.E. Consortium Clone ID numbers and the corresponding GenBank accession numbers of sequences identified as belonging to the I.M.A.G.E. Consortium and UniGene clusters, are listed below. Also included are sequences that are not identified as having a Clone ID number but still identified as being those of QPRT. The sequences include those of the “sense” and complementary strands sequences corresponding to QPRT. Representative sequences of GenBank accession numbers below are presented in the attached Appendix to PCT/US04/30789.

Clone ID number/GenBank accession number(s)

2960170/BC005060 and BE299670 and BE299712 □
3506460/BE273102 and BC010033 □
3959973/BC018910 andBE902622 □
267692/N23182 andN32648 □
3843834/BE735342 □
4872092/BX283118 and BG769505 □
4845859/BG750434 □
4868806/BG766440 □
4594651/BG401877 □
4553618/BG337811 □
4554044/BG338063 □
4473161/BG251163 □
4581127/BG396079 □
4136221/BF316915 □
4127089/BF313098 □
4508387/BG257831 □
4125826/BF312975 □
4416920/BG115486
4842556/BG748194
4395232/BF980859
4122808/BF304964
6305325/BQ643384
4107138/BF204965
4875437/BG753310/
6337913/BU501237
4136491/BF317004
4131857/BF307788
4302204/BF684687
5092370/BI195027
3353576/BE257622
4473768/BG252578
3912491/BE887856
6012403/BU186666
4873695/BG751315
4873694/BG751234
4080072/BF237708
6300166/BQ876922
896716/BI198351
4877853/BG770209
4896715/BI198375
5087154/BI252426 and BI252874
6085289/BU174626
5741237/BM558378
4995462/BI088884
6720160/CA488404
5764841/BM926410
6208509/BQ879962
4581968/BG396587
5554997/BM477735
3451668/BE538581
5803440/BQ069150
6250974/BQ688755
6251079/BQ685759
5535758/BM468306
6146330/BU165540
1740729/AI191477
2729947/AW293885
6082577/BU174653
2753118/AW275889
2437568/AI884372
2507497/AI961218
5207705/BI771713
2067750/AI383718
263894/N28522 and H99843
1148416/AA627205
138014/R63144
70610/T49073 and T49074
5531252/BM800219
3629874/BE409186
5001398/BI093643
4361336/BF971224
4451023/BG121013
3844815/BE730924
4361451/BF970190
4154033/BF346117
4915206/BG818225
4444686/BG118070
6086243/BU149745
4899066/BG829478
6086128/BU180123
4366207/BG108477
3140355/BE280221
5459527/BM012505
3627907/BE382922
5418599/BM016313
4862852/BG765156
4877780/BG769917
3162024/BE262076
5182393/BI518189 and BI517759
5417445/BM015407
6015713/BU175170
417111/W87557 and W87461
4580196/BG395022
6271908/BQ648651
6298174/BQ652789
6271910/BQ653475
6271630/BQ647246
5798664/BM928534
6652195/BU860925
6299767/BQ651366
5225259/BI838658
4895399/BI198873
740128/AA477534 and AA479051
6172561/BU178924
4562784/BG326197
3957711/BE902093
6293406/BQ650920
None (mRNA sequences)/BT007231.1 and NM_014298.2
and AK090801.1 and D78177.1
None/CB156177.1 and BM711970.1 and BM675916.1
and BM675420.1 and BM714918.1 and
AA337770.1 and AA305670.1 and AA305611.1 and
and BU622082.1 and AV705250.1 and
AL528086.2 and AL531128.2 and AL543783.2 and
AL548817.2 and AL554386.2 and AL563056.2
and AL563955.2 and AL570131.2 and AL573234.2
and BF956608.1 and AV648116.1 and
AV645766.1 and BF742969.1 and AL577191.2 and
CD050133.1 and CD049103.1 and BX417895.1
and CB529044.1 and CD250136.1 and AA054830.1
and BX508036.1 and BX454610.1

In another set of preferred embodiments of the invention, any sequence, or unique portion thereof, of the HOXB13 sequences of the I.M.A.G.E. Consortium cluster NM_{—}006361, as well as the UniGene Homo sapiens cluster Hs.66731, may be used. Similarly, any sequence encoding all or a part of the protein encoded by any HOXB13 sequence disclosed herein may be used. The consensus sequence of the I.M.A.G.E. Consortium cluster is as follows, with the assigned coding region (ending with a termination codon) underlined and preceded by the 5′ untranslated and/or non-coding region and followed by the 3′ untranslated and/or non-coding region:

SEQ ID NO:6 (consensus sequence for HOXB13, identified as NM_{—}006361 or NM_{—}006361.2):

cgaatgcagg cgacttgcga gctgggagcg atttaaaacg ctttggattc ccccggcctg
ggtggggaga gcgagctggg tgccccctag attccccgcc cccgcacctc atgagccgac
cctcggctcc atggagcccg gcaattatgc caccttggat ggagccaagg atatcgaagg
cttgctggga gcgggagggg ggcggaatct ggtcgcccac tcccctctga ccagccaccc
agcggcgcct acgctgatgc ctgctgtcaa ctatgccccc ttggatctgc caggctcggc
ggagccgcca aagcaatgcc acccatgccc tggggtgccc caggggacgt ccccagctcc
cgtgccttat ggttactttg gaggcgggta ctactcctgc cgagtgtccc ggagctcgct
gaaaccctgt gcccaggcag ccaccctggc cgcgtacccc gcggagactc ccacggccgg
ggaagagtac cccagtcgcc ccactgagtt tgccttctat ccgggatatc cgggaaccta
ccacgctatg gccagttacc tggacgtgtc tgtggtgcag actctgggtg ctcctggaga
accgcgacat gactccctgt tgcctgtgga cagttaccag tcttgggctc tcgctggtgg
ctggaacagc cagatgtgtt gccagggaga acagaaccca ccaggtccct tttggaaggc
agcatttgca gactccagcg ggcagcaccc tcctgacgcc tgcgcctttc gtcgcggccg
caagaaacgc attccgtaca gcaaggggca gttgcgggag ctggagcggg agtatgcggc
taacaagttc atcaccaagg acaagaggcg caagatctcg gcagccacca gcctctcgga
gcgccagatt accatctggt ttcagaaccg ccgggtcaaa gagaagaagg ttctcgccaa
ggtgaagaac agcgctaccc cttaagagat ctccttgcct gggtgggagg agcgaaagtg
ggggtgtcct ggggagacca gaaacctgcc aagcccaggc tggggccaag gactctgctg
agaggcccct agagacaaca cccttcccag gccactggct gctggactgt tcctcaggag
cggcctgggt acccagtatg tgcagggaga cggaacccca tgtgacaggc ccactccacc
agggttccca aagaacctgg cccagtcata atcattcatc ctcacagtgg caataatcac
gataaccagt

I.M.A.G.E. Consortium Clone ID numbers and the corresponding GenBank accession numbers of sequences identified as belonging to the I.M.A.G.E. Consortium and UniGene clusters, are listed below. Also included are sequences that are not identified as having a Clone ID number but still identified as being those of HOXB13. The sequences include those of the “sense” and complementary strands sequences corresponding to HOXB13. The sequence of each GenBank accession number is presented in the attached Appendix to PCT/US04/30789.

| Clone ID | |
|---|---|
| numbers | GenBank accession numbers |
| 4250486 | BF676461, BC007092 |
| 5518335 | BM462617 |
| 4874541 | BG752489 |
| 4806039 | BG778198 |
| 3272315 | CB050884, CB050885 |
| 4356740 | BF965191 |
| 6668163 | BU930208 |
| 1218366 | AA807966 |
| 2437746 | AI884491 |
| 1187697 | AA652388 |
| 3647557 | BF446158 |
| 1207949 | AA657924 |
| 1047774 | AA644637 |
| 3649397 | BF222357 |
| 971664 | AA527613 |
| 996191 | AA533227 |
| 813481 | AA456069, AA455572, BX117624 |
| 6256333 | BQ673782 |
| 2408470 | AI814453 |
| 2114743 | AI417272 |
| 998548 | AA535663 |
| 2116027 | AI400493 |
| 3040843 | AW779219 |
| 1101311 | AA594847 |
| 1752062 | AI150430 |
| 898712 | AA494387 |
| 1218874 | AA662643 |
| 2460189 | AI935940 |
| 986283 | AA532530 |
| 1435135 | AA857572 |
| 1871750 | AI261980 |
| 3915135 | BE888751 |
| 2069668 | AI378797 |
| 667188 | AA234220, AA236353 |
| 1101561 | AA588193 |
| 1170268 | AI821103, AI821851, AA635855 |
| 2095067 | AI420753 |
| 4432770 | BG180547 |
| 783296 | AA468306, AA468232 |
| 3271646 | CB050115, CB050116 |
| 1219276 | AA661819 |
| 30570598 | CF146837 |
| 30570517 | CF146763 |
| 30568921 | CF144902 |
| 3099071 | CF141511 |
| 3096992 | CF139563 |
| 3096870 | CF139372 |
| 3096623 | CF139319 |
| 3096798 | CF139275 |
| 30572408 | CF122893 |
| 2490082 | AI972423 |
| 2251055 | AI918975 |
| 2419308 | AI826991 |
| 2249105 | AI686312 |
| 2243362 | AI655923 |
| 30570697 | CF146922 |
| 3255712 | BF476369 |
| 3478356 | BF057410 |
| 3287977 | BE645544 |
| 3287746 | BE645408 |
| 3621499 | BE388501 |
| 30571128 | CF147366 |
| 30570954 | CF147143 |
| None (mRNA | BT007410, BC007092, U57052, U81599 |
| sequences) | |
| None | CB120119, CB125764, AU098628, CB126130, |
| | BI023924, BM767063, BM794275, BQ363211, |
| | BM932052, AA357646, AW609525, CB126919, |
| | AW609336, AW609244, BF855145, AU126914, |
| | CB126449, AW582404, BX641644 |

In one preferred embodiment, any sequence, or unique portion thereof, of the following HOXB13 sequence, identified by BC007092 or BC007092.1, may be used in the practice of the invention.

SEQ ID NO:7 (sequence for HOXB13):

GGATTCCCCCGGCCTGGGTGGGGAGAGCGAGCTGGGTGCCCCCTAGATTCCCCGCCCCCG
CACCTCATGAGCCGACCCTCGGCTCCATGGAGCCCGGCAATTATGCCACCTTGGATGGAG
CCAAGGATATCGAAGGCTTGCTGGGAGCGGGAGGGGGGCGGAATCTGGTCGCCCACTCCC
CTCTGACCAGCCACCCAGCGGCGCCTACGCTGATGCCTGCTGTCAACTATGCCCCCTTGG
ATCTGCCAGGCTCGGCGGAGCCGCCAAAGCAATGCCACCCATGCCCTGGGGTGCCCCAGG
GGACGTCCCCAGCTCCCGTGCCTTATGGTTACTTTGGAGGCGGGTACTACTCCTGCCGAG
TGTCCCGGAGCTCGCTGAAACCCTGTGCCCAGGCAGCCACCCTGGCCGCGTACCCCGCGG
AGACTCCCACGGCCGGGGAAGAGTACCCCAGCCGCCCCACTGAGTTTGCCTTCTATCCGG
GATATCCGGGAACCTACCAGCCTATGGCCAGTTACCTGGACGTGTCTGTGGTGCAGACTC
TGGGTGCTCCTGGAGAACCGCGACATGACTCCCTGTTGCCTGTGGACAGTTACCAGTCTT
GGGCTCTCGCTGGTGGCTGGAACAGCCAGATGTGTTGCCAGGGAGAACAGAACCCACCAG
GTCCCTTTTGGAAGGCAGCATTTGCAGACTCCAGCGGGCAGCACCCTCCTGACGCCTGCG
CCTTTCGTCGCGGCCGCAAGAAACGCATTCCGTACAGCAAGGGGCAGTTGCGGGAGCTGG
AGCGGGAGTATGCGGCTAACAAGTTCATCACCAAGGACAAGAGGCGCAAGATCTCGGCAG
CCACCAGCCTCTCGGAGCGCCAGATTACCATCTGGTTTCAGAACCGCCGGGTCAAAGAGA
AGAAGGTTCTCGCCAAGGTGAAGAACAGCGCTACCCCTTAAGAGATCTCCTTGCCTGGGT
GGGAGGAGCGAAAGTGGGGGTGTCCTGGGGAGACCAGGAACCTGCCAAGCCCAGGCTGGG
GCCAAGGACTCTGCTGAGAGGCCCCTAGAGACAACACCCTTCCCAGGCCACTGGCTGCTG
GACTGTTCCTCAGGAGCGGCCTGGGTACCCAGTATGTGCAGGGAGACGGAACCCCATGTG
ACAGCCCACTCCACCAGGGTTCCCAAAGAACCTGGCCCAGTCATAATCATTCATCCTGAC
AGTGGCAATAATCACGATAACCAGTACTAGCTGCCATGATCGTTAGCCTCATATTTTCTA
TCTAGAGCTCTGTAGAGCACTTTAGAAACCGCTTTCATGAATTGAGCTAATTATGAATAA
ATTTGGAAAAAAAAAAAAAAAAAAAAAAAAAAAAAA

Sequences identified by SEQ ID NO. are provided using conventional representations of a DNA strand starting from the 5′ phosphate linked end to the 3′ hydroxyl linked end. The assignment of coding regions is generally by comparison to available consensus sequence(s) and therefore may contain inconsistencies relative to other sequences assigned to the same cluster. These have no effect on the practice of the invention because the invention can be practiced by use of shorter segments (or combinations thereof) of sequences unique to each of the three sets described above and not affected by inconsistencies. As non-limiting examples, a segment of IL17BR, CHDH, QPRT, or HOXB13 nucleic acid sequence composed of a 3′ untranslated region sequence and/or a sequence from the 3′ end of the coding region may be used as a probe for the detection of IL17BR, CHDH, QPRT, or HOXB13 expression, respectively, without being affected by the presence of any inconsistency in the coding regions due to differences between sequences. Similarly, the use of an antibody which specifically recognizes a protein, or fragment thereof, encoded by the IL17BR, CHDH, QPRT, or HOXB13 sequences described herein, to detect its expression would not be affected by the presence of any inconsistency in the representation of the coding regions provided above.

As will be appreciated by those skilled in the art, some of the above sequences include 3′ poly A (or poly T on the complementary strand) stretches that do not contribute to the uniqueness of the disclosed sequences. The invention may thus be practiced with sequences lacking the 3′ poly A (or poly T) stretches. The uniqueness of the disclosed sequences refers to the portions or entireties of the sequences which are found only in IL17BR, CHDH, QPRT, or HOXB13 nucleic acids, including unique sequences found at the 3′ untranslated portion of the genes. Preferred unique sequences for the practice of the invention are those which contribute to the consensus sequences for each of the three sets such that the unique sequences will be useful in detecting expression in a variety of individuals rather than being specific for a polymorphism present in some individuals. Alternatively, sequences unique to an individual or a subpopulation may be used. The preferred unique sequences are preferably of the lengths of polynucleotides of the invention as discussed herein.

To determine the (increased or decreased) expression levels of the above described sequences in the practice of the present invention, any method known in the art may be utilized. In one preferred embodiment of the invention, expression based on detection of RNA which hybridizes to polynucleotides containing the above described sequences is used. This is readily performed by any RNA detection or amplification+detection method known or recognized as equivalent in the art such as, but not limited to, reverse transcription-PCR (optionally real-time PCR), the methods disclosed in U.S. Pat. No. 6,794,141, the methods disclosed in U.S. Pat. No. 6,291,170, and quantitative PCR. Methods to identify increased RNA stability (resulting in an observation of increased expression) or decreased RNA stability (resulting in an observation of decreased expression) may also be used. These methods include the detection of sequences that increase or decrease the stability of mRNAs containing the IL17BR, CHDH, QPRT, or HOXB13 sequences disclosed herein. These methods also include the detection of increased mRNA degradation.

In particularly preferred embodiments of the invention, polynucleotides having sequences present in the 3′ untranslated and/or non-coding regions of the above disclosed sequences are used to detect expression or non-expression of IL17BR, CHDH, QPRT, or HOXB13 sequences in breast cells in the practice of the invention. Such polynucleotides may optionally contain sequences found in the 3′ portions of the coding regions of the above disclosed sequences. Polynucleotides containing a combination of sequences from the coding and 3′ non-coding regions preferably have the sequences arranged contiguously, with no intervening heterologous sequence(s).

Alternatively, the invention may be practiced with polynucleotides having sequences present in the 5′ untranslated and/or non-coding regions of IL17BR, CHDH, QPRT, or HOXB13 sequences in breast cells to detect their levels of expression. Such polynucleotides may optionally contain sequences found in the 5′ portions of the coding regions. Polynucleotides containing a combination of sequences from the coding and 5′ non-coding regions preferably have the sequences arranged contiguously, with no intervening heterologous sequence(s). The invention may also be practiced with sequences present in the coding regions of 17BR, CHDH, QPRT, or HOXB13.

Preferred polynucleotides contain sequences from 3′ or 5′ untranslated and/or non-coding regions of at least about 16, at least about 18, at least about 20, at least about 22, at least about 24, at least about 26, at least about 28, at least about 30, at least about 32, at least about 34, at least about 36, at least about 38, at least about 40, at least about 42, at least about 44, or at least about 46 consecutive nucleotides. The term “about” as used in the previous sentence refers to an increase or decrease of 1 from the stated numerical value. Even more preferred are polynucleotides containing sequences of at least or about 50, at least or about 100, at least about or 150, at least or about 200, at least or about 250, at least or about 300, at least or about 350, or at least or about 400 consecutive nucleotides. The term “about” as used in the preceding sentence refers to an increase or decrease of 10% from the stated numerical value.

Sequences from the 3′ or 5′ end of the above described coding regions as found in polynucleotides of the invention are of the same lengths as those described above, except that they would naturally be limited by the length of the coding region. The 3′ end of a coding region may include sequences up to the 3′ half of the coding region. Conversely, the 5′ end of a coding region may include sequences up the 5′ half of the coding region. Of course the above described sequences, or the coding regions and polynucleotides containing portions thereof, may be used in their entireties.

Polynucleotides combining the sequences from a 3′ untranslated and/or non-coding region and the associated 3′ end of the coding region are preferably at least or about 100, at least about or 150, at least or about 200, at least or about 250, at least or about 300, at least or about 350, or at least or about 400 consecutive nucleotides. Preferably, the polynucleotides used are from the 3′ end of the gene, such as within about 350, about 300, about 250, about 200, about 150, about 100, or about 50 nucleotides from the polyadenylation signal or polyadenylation site of a gene or expressed sequence. Polynucleotides containing mutations relative to the sequences of the disclosed genes may also be used so long as the presence of the mutations still allows hybridization to produce a detectable signal.

In another embodiment of the invention, polynucleotides containing deletions of nucleotides from the 5′ and/or 3′ end of the above disclosed sequences may be used. The deletions are preferably of 1-5, 5-10, 10-15, 15-20, 20-25, 25-30, 30-35, 35-40, 40-45, 45-50, 50-60, 60-70, 70-80, 80-90, 90-100, 100-125, 125-150, 150-175, or 175-200 nucleotides from the 5′ and/or 3′ end, although the extent of the deletions would naturally be limited by the length of the disclosed sequences and the need to be able to use the polynucleotides for the detection of expression levels.

Other polynucleotides of the invention from the 3′ end of the above disclosed sequences include those of primers and optional probes for quantitative PCR. Preferably, the primers and probes are those which amplify a region less than about 350, less than about 300, less than about 250, less than about 200, less than about 150, less than about 100, or less than about 50 nucleotides from the from the polyadenylation signal or polyadenylation site of a gene or expressed sequence.

In yet another embodiment of the invention, polynucleotides containing portions of the above disclosed sequences including the 3′ end may be used in the practice of the invention. Such polynucleotides would contain at least or about 50, at least or about 100, at least about or 150, at least or about 200, at least or about 250, at least or about 300, at least or about 350, or at least or about 400 consecutive nucleotides from the 3′ end of the disclosed sequences.

The invention thus also includes polynucleotides used to detect IL17BR, CHDH, QPRT, or HOXB13 expression in breast cells. The polynucleotides may comprise a shorter polynucleotide consisting of sequences found in the above provided SEQ ID NOS in combination with heterologous sequences not naturally found in combination with IL17BR, CHDH, QPRT, or HOXB13 sequences.

As non-limiting examples, a polynucleotide comprising one of the following sequences may be used in the practice of the invention.

SEQ ID NO: 8:
GCTCTCACTGGCAAATGACAGCTCTGTGCAAGGAGCACTCCCAAGTATAAAAATTATTAC
SEQ ID NO: 9:
TGCCTAATTTCACTCTCAGAGTGAGGCAGGTAACTGGGGCTCCACTGGGTCACTCTGAGA
SEQ ID NO: 10:
GATCGTTAGCCTCATATTTTCTATCTAGAGCTCTGTAGAGCACTTTAGAAACCGCTTTCA

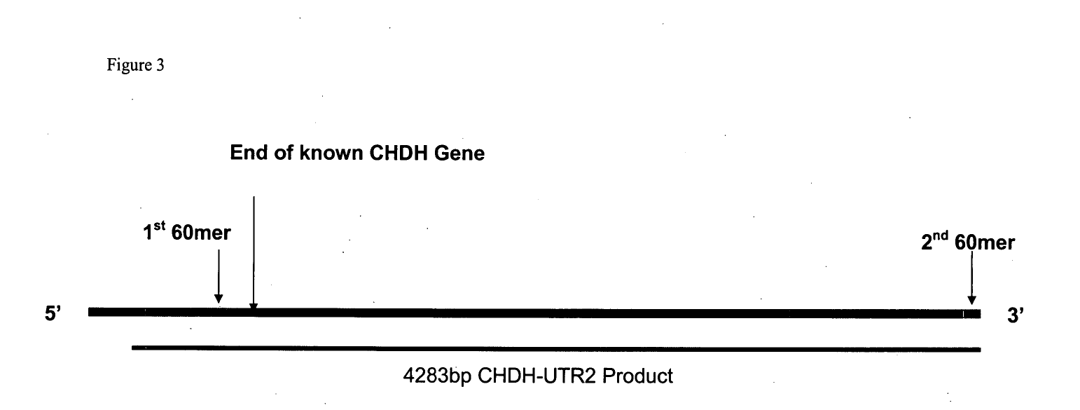

SEQ ID NO:8 is a portion of the AI240933 sequence while SEQ ID NO:9 is a portion of the AJ272267 (CHDH mRNA) sequence. They correspond to the two “60mer” positions indicated in FIG. 3. SEQ ID NO:10 is a polynucleotide capable of hybridizing to some HOXB13 sequences as described herein.

Thus, the invention may be practiced with a polynucleotide consisting of the sequence of SEQ ID NOS:8, 9 or 10 in combination with one or more heterologous sequences that are not normally found with SEQ ID NOS:8, 9 or 10. Alternatively, the invention may also be practiced with a polynucleotide consisting of the sequence of SEQ ID NOS:8, 9 or 10 in combination with one or more naturally occurring sequences that are normally found with SEQ ID NOS:8, 9 or 10.

Polynucleotides with sequences comprising SEQ ID NOS:8 or 9, either naturally occurring or synthetic, may be used to detect nucleic acids which are over expressed in breast cancer cells that are responsive, and those which are not over expressed in breast cancer cells that are non-responsive, to treatment with TAM or another “antiestrogen” agent against breast cancer. Polynucleotides with sequences comprising SEQ ID NO:10, either naturally occurring or synthetic, may be used to detect nucleic acids which are under expressed in breast cancer cells that are responsive, and those which are not under expressed in breast cancer cells that are non-responsive, to treatment with TAM or another “antiestrogen” agent against breast cancer.

Additional sequences that may be used in polynucleotides as described above for SEQ ID NOS:8 and 9 is the following, which is complementary to a portion of IL17BR sequences disclosed herein:

| | |
|---|---|
| SEQ ID NO: 11: | TCCAATCGTTAGTTAATGCTACATTAGTT |

Additional sequences that may be used in polynucleotides as described above for SEQ ID NO:10 are the following, which is complementary to a portion of IL17BR sequences disclosed herein:

| | |
|---|---|
| SEQ ID NO: 12: | CAATTCATGAAAGCGGTTTCTAAAG |

Additionally, primers of defined sequences may be used to PCR amplify portions of CHDH sequences to determine their level of expression. For example, primers comprising the following sequences may be used to amplify a portion of the AI240933 sequence.

| | |
|---|---|
| Forward primer: | |
| (SEQ ID NO: 13) | TGAAGTGTTTTTGCCTGGATCA |
| Reverse primer: | |
| (SEQ ID NO: 14) | CACCACTTTGTTATGAAGACCTTACAA |

In some embodiments of the invention, the primers may be used in quantitative RT-PCR methods known in the art, optionally in the presence of a labeled or detectable probe that binds double stranded nucleic acids (such as Sybr Green™) or a specific probe such as a “TaqMan” probe. In one embodiment, such a probe may comprise the sequence AGTAAGAATGTCTTAAGAAGAGG (SEQ ID NO:15) for the detection of AI240933 expression.

Additionally, polynucleotides containing other sequences, particularly unique sequences, present in naturally occurring nucleic acid molecules comprising SEQ ID NOS:8-15 may be used in the practice of the invention.

Other polynucleotides for use in the practice of the invention include those that have sufficient homology to those described above to detect expression by use of hybridization techniques. Such polynucleotides preferably have about or 95%, about or 96%, about or 97%, about or 98%, or about or 99% identity with IL17BR, CHDH, QPRT, or HOXB13 sequences as described herein. Identity is determined using the BLAST algorithm, as described above. The other polynucleotides for use in the practice of the invention may also be described on the basis of the ability to hybridize to polynucleotides of the invention under stringent conditions of about 30% v/v to about 50% formamide and from about 0.01M to about 0.15M salt for hybridization and from about 0.01M to about 0.15M salt for wash conditions at about 55 to about 65° C. or higher, or conditions equivalent thereto.

In a further embodiment of the invention, a population of single stranded nucleic acid molecules comprising one or both strands of a human IL17BR, CHDH, QPRT, or HOXB13 sequence is provided as a probe such that at least a portion of said population may be hybridized to one or both strands of a nucleic acid molecule quantitatively amplified from RNA of a breast cancer cell. The population may be only the antisense strand of a human IL17BR, CHDH, QPRT, or HOXB13 sequence such that a sense strand of a molecule from, or amplified from, a breast cancer cell may be hybridized to a portion of said population. In the case of IL17BR or CHDH, the population preferably comprises a sufficiently excess amount of said one or both strands of a human IL17BR or CHDH sequence in comparison to the amount of expressed (or amplified) nucleic acid molecules containing a complementary IL17BR or CHDH sequence from a normal breast cell. This condition of excess permits the increased amount of nucleic acid expression in a breast cancer cell to be readily detectable as an increase.

Alternatively, the population of single stranded molecules is equal to or in excess of all of one or both strands of the nucleic acid molecules amplified from a breast cancer cell such that the population is sufficient to hybridize to all of one or both strands. Preferred cells are those of a breast cancer patient that is ER+ or for whom treatment with tamoxifen or one or more other “antiestrogen” agent against breast cancer is contemplated. The single stranded molecules may of course be the denatured form of any IL17BR, CHDH, QPRT, or HOXB13 sequence containing double stranded nucleic acid molecule or polynucleotide as described herein.

The population may also be described as being hybridized to an IL17BR or CHDH sequence containing nucleic acid molecules at a level of at least twice as much as that by nucleic acid molecules of a normal breast cell. As in the embodiments described above, the nucleic acid molecules may be those quantitatively amplified from a breast cancer cell such that they reflect the amount of expression in said cell.

The population is preferably immobilized on a solid support, optionally in the form of a location on a microarray. A portion of the population is preferably hybridized to nucleic acid molecules quantitatively amplified from a non-normal or abnormal breast cell by RNA amplification. The amplified RNA may be that derived from a breast cancer cell, as long as the amplification used was quantitative with respect to IL17BR, CHDH, QPRT, or HOXB13 containing sequences.

In another embodiment of the invention, expression based on detection of DNA status may be used. Detection of the QPRT or HOXB13 DNA as methylated, deleted or otherwise inactivated, may be used as an indication of decreased expression as found in non-normal breast cells. This may be readily performed by PCR based methods known in the art. The status of the promoter regions of QPRT or HOXB13 may also be assayed as an indication of decreased expression of QPRT or HOXB13 sequences. A non-limiting example is the methylation status of sequences found in the promoter region.

Conversely, detection of the DNA of a sequence as amplified may be used for as an indication of increased expression as found in non-normal breast cells. This may be readily performed by PCR based, fluorescent in situ hybridization (FISH) and chromosome in situ hybridization (CISH) methods known in the art.

A preferred embodiment using a nucleic acid based assay to determine expression is by immobilization of one or more of the sequences identified herein on a solid support, including, but not limited to, a solid substrate as an array or to beads or bead based technology as known in the art. Alternatively, solution based expression assays known in the art may also be used. The immobilized sequence(s) may be in the form of polynucleotides as described herein such that the polynucleotide would be capable of hybridizing to a DNA or RNA corresponding to the sequence(s).

The immobilized polynucleotide(s) may be used to determine the state of nucleic acid samples prepared from sample breast cancer cell(s), optionally as part of a method to detect ER status in said cell(s). Without limiting the invention, such a cell may be from a patient suspected of being afflicted with, or at risk of developing, breast cancer. The immobilized polynucleotide(s) need only be sufficient to specifically hybridize to the corresponding nucleic acid molecules derived from the sample (and to the exclusion of detectable or significant hybridization to other nucleic acid molecules).

In yet another embodiment of the invention, a ratio of the expression levels of two of the disclosed genes may be used to predict response to treatment with TAM or another SERM. Preferably, the ratio is that of two genes with opposing patterns of expression, such as an underexpressed gene to an overexpressed gene, in correlation to the same phenotype. Non-limiting examples include the ratio of HOXB13 over IL17BR or the ratio of QPRT over CHDH. Of course the use of the inverse of each of these ratios would be recognized by the skilled person as suitable for the practice of the invention. This aspect of the invention is based in part on the observation that such a ratio has a stronger correlation with TAM treatment outcome than the expression level of either gene alone. For example, the ratio of HOXB13 over IL17BR has an observed classification accuracy of 77%. Alternatively, a ratio of expression of any one of HOXB13, IL17BR, CHDH, and QPRT to the expression of a reference gene, to detect over or underexpression as disclosed herein, may be used.

Where a HOXB13 to IL17BR ratio is used, a value greater than about 1.7, about 1.75, about 1.8 about 1.85, or about 1.9 is expected to indicate a significantly worse disease-free survival and overall survival independent of tumor size, nodal status, tumor grade and HER-2 overexpression compared with women with a lower ratio.

As a non-limiting example, the Ct values from Q-PCR based detection of gene expression levels may be used to derive a ratio to predict the response to treatment with one or more “antiestrogen” agent against breast cancer. As would be recognized by the skilled person, the Ct values may be determined by a variety of methods, including, but not limited to, standardization against a standard curve with cDNA dilutions derived from amplification of a reference sequence, such as human universal total RNA (Stratagene).

### ADDITIONAL EMBODIMENTS OF THE INVENTION

In embodiments where only one or a few genes are to be analyzed, the nucleic acid derived from the sample breast cancer cell(s) may be preferentially amplified by use of appropriate primers such that only the genes to be analyzed are amplified to reduce contaminating background signals from other genes expressed in the breast cell. Alternatively, and where multiple genes are to be analyzed or where very few cells (or one cell) is used, the nucleic acid from the sample may be globally amplified before hybridization to the immobilized polynucleotides. Of course RNA, or the cDNA counterpart thereof may be directly labeled and used, without amplification, by methods known in the art.

Sequence expression based on detection of a presence, increase, or decrease in protein levels or activity may also be used. Detection may be performed by any immunohistochemistry (IHC) based, bodily fluid based (where a IL17BR, CHDH, QPRT, or HOXB13 polypeptide, or fragment thereof, is found in a bodily fluid, such as but not limited to blood), antibody (including autoantibodies against the protein where present) based, ex foliate cell (from the cancer) based, mass spectroscopy based, and image (including used of labeled ligand where available) based method known in the art and recognized as appropriate for the detection of the protein. Antibody and image based methods are additionally useful for the localization of tumors after determination of cancer by use of cells obtained by a non-invasive procedure (such as ductal lavage or fine needle aspiration), where the source of the cancerous cells is not known. A labeled antibody or ligand may be used to localize the carcinoma(s) within a patient.

Antibodies for use in such methods of detection include polyclonal antibodies, optionally isolated from naturally occurring sources where available, and monoclonal antibodies, including those prepared by use of IL17BR, CHDH, QPRT, or HOXB13 polypeptides (or fragments thereof) as antigens. Such antibodies, as well as fragments thereof (including but not limited to F_{ab} fragments) function to detect or diagnose non-normal or cancerous breast cells by virtue of their ability to specifically bind IL17BR, CHDH, QPRT, or HOXB13 polypeptides to the exclusion of other polypeptides to produce a detectable signal. Recombinant, synthetic, and hybrid antibodies with the same ability may also be used in the practice of the invention. Antibodies may be readily generated by immunization with a IL17BR, CHDH, QPRT, or HOXB13 polypeptide (or fragment thereof), and polyclonal sera may also be used in the practice of the invention.

Antibody based detection methods are well known in the art and include sandwich and ELISA assays as well as Western blot and flow cytometry based assays as non-limiting examples. Samples for analysis in such methods include any that contain IL17BR, CHDH, QPRT, or HOXB13 polypeptides or fragments thereof. Non-limiting examples include those containing breast cells and cell contents as well as bodily fluids (including blood, serum, saliva, lymphatic fluid, as well as mucosal and other cellular secretions as non-limiting examples) that contain the polypeptides.

The above assay embodiments may be used in a number of different ways to identify or detect the response to treatment with TAM or another “antiestrogen” agent against breast cancer based on gene expression in a breast cancer cell sample from a patient. In some cases, this would reflect a secondary screen for the patient, who may have already undergone mammography or physical exam as a primary screen. If positive from the primary screen, the subsequent needle biopsy, ductal lavage, fine needle aspiration, or other analogous minimally invasive method may provide the sample for use in the assay embodiments before, simultaneous with, or after assaying for ER status. The present invention is particularly useful in combination with non-invasive protocols, such as ductal lavage or fine needle aspiration, to prepare a breast cell sample.

The present invention provides a more objective set of criteria, in the form of gene expression profiles of a discrete set of genes, to discriminate (or delineate) between breast cancer outcomes. In particularly preferred embodiments of the invention, the assays are used to discriminate between good and poor outcomes after treatment with tamoxifen or another “antiestrogen” agent against breast cancer. Comparisons that discriminate between outcomes after about 10, about 20, about 30, about 40, about 50, about 60, about 70, about 80, about 90, about 100, or about 150 months may be performed.

While good and poor survival outcomes may be defined relatively in comparison to each other, a “good” outcome may be viewed as a better than 50% survival rate after about 60 months post surgical intervention to remove breast cancer tumor(s). A “good” outcome may also be a better than about 60%, about 70%, about 80% or about 90% survival rate after about 60 months post surgical intervention. A “poor” outcome may be viewed as a 50% or less survival rate after about 60 months post surgical intervention to remove breast cancer tumor(s). A “poor” outcome may also be about a 70% or less survival rate after about 40 months, or about a 80% or less survival rate after about 20 months, post surgical intervention.

In another embodiment of the invention based on the expression of a few genes, the isolation and analysis of a breast cancer cell sample may be performed as follows:
(1) Ductal lavage or other non-invasive procedure is performed on a patient to obtain a sample.
(2) Sample is prepared and coated onto a microscope slide. Note that ductal lavage results in clusters of cells that are cytologically examined as stated above.
(3) Pathologist or image analysis software scans the sample for the presence of atypical cells.
(4) If atypical cells are observed, those cells are harvested (e.g. by microdissection such as LCM).
(5) RNA is extracted from the harvested cells.
(6) RNA is assayed, directly or after conversion to cDNA or amplification therefrom, for the expression of IL17BR, CHDH, QPRT, and/or HOXB13 sequences.

With use of the present invention, skilled physicians may prescribe or withhold treatment with TAM or another “antiestrogen” agent against breast cancer based on prognosis determined via practice of the instant invention.

The above discussion is also applicable where a palpable lesion is detected followed by fine needle aspiration or needle biopsy of cells from the breast. The cells are plated and reviewed by a pathologist or automated imaging system which selects cells for analysis as described above.

The present invention may also be used, however, with solid tissue biopsies, including those stored as an FFPE specimen. For example, a solid biopsy may be collected and prepared for visualization followed by determination of expression of one or more genes identified herein to determine the breast cancer outcome. As another non-limiting example, a solid biopsy may be collected and prepared for visualization followed by determination of IL17BR, CHDH, QPRT, and/or HOXB13 expression. One preferred means is by use of in situ hybridization with polynucleotide or protein identifying probe(s) for assaying expression of said gene(s).

In an alternative method, the solid tissue biopsy may be used to extract molecules followed by analysis for expression of one or more gene(s). This provides the possibility of leaving out the need for visualization and collection of only cancer cells or cells suspected of being cancerous. This method may of course be modified such that only cells that have been positively selected are collected and used to extract molecules for analysis. This would require visualization and selection as a prerequisite to gene expression analysis. In the case of an FFPE sample, cells may be obtained followed by RNA extraction, amplification and detection as described herein.

In an alternative to the above, the sequence(s) identified herein may be used as part of a simple PCR or array based assay simply to determine the response to treatment with TAM or another “antiestrogen” agent against breast cancer by use of a sample from a non-invasive or minimally invasive sampling procedure. The detection of sequence expression from samples may be by use of a single microarray able to assay expression of the disclosed sequences as well as other sequences, including sequences known not to vary in expression levels between normal and non-normal breast cells, for convenience and improved accuracy.

Other uses of the present invention include providing the ability to identify breast cancer cell samples as having different responses to treatment with TAM or another “antiestrogen” agent against breast cancer for further research or study. This provides an advance based on objective genetic/molecular criteria.

In yet another embodiment of the invention based on the expression of multiple genes in an expression pattern or profile, the isolation and analysis of a breast cancer cell sample may be performed as follows:
(1) Ductal lavage or other non-invasive procedure is performed on a patient to obtain a sample.
(2) Sample is prepared and coated onto a microscope slide. Note that ductal lavage results in clusters of cells that are cytologically examined as stated above.
(3) Pathologist or image analysis software scans the sample for the presence of non-normal and/or atypical breast cancer cells.
(4) If such cells are observed, those cells are harvested (e.g. by microdissection such as LCM).
(5) RNA is extracted from the harvested cells.
(6) RNA is purified, amplified, and labeled.
(7) Labeled nucleic acid is contacted with a microarray containing polynucleotides complementary to all or part of one or more of the genes identified herein as correlated to discriminations between breast cancer outcomes under suitable hybridization conditions, then processed and scanned to obtain a pattern of intensities of each spot (relative to a control for general gene expression in cells) which determine the level of expression of the gene(s) in the cells.
(8) The pattern of intensities is analyzed by comparison to the expression patterns of the genes in known samples of breast cancer cells correlated with outcomes (relative to the same control).

A specific example of the above method would be performing ductal lavage following a primary screen, observing and collecting non-normal and/or atypical cells for analysis. The comparison to known expression patterns, such as that made possible by a model generated by an algorithm (such as, but not limited to nearest neighbor type analysis, SVM, or neural networks) with reference gene expression data for the different breast cancer survival outcomes, identifies the cells as being correlated with subjects with good or poor outcomes. Another example would be taking a breast tumor removed from a subject after surgical intervention, optionally converting all or part of it to an FFPE sample prior to subsequent isolation and preparation of breast cancer cells from the tumor for determination/identification of atypical, non-normal, or cancer cells, and isolation of said cells followed by steps 5 through 8 above.

Alternatively, the sample may permit the collection of both normal as well as cancer cells for analysis. The gene expression patterns for each of these two samples will be compared to each other as well as the model and the normal versus individual comparisons therein based upon the reference data set. This approach can be significantly more powerful that the cancer cells only approach because it utilizes significantly more information from the normal cells and the differences between normal and cancer cells (in both the sample and reference data sets) to determine the breast cancer outcome of the patient based on gene expression in the cancer cells from the sample.

The genes identified herein also may be used to generate a model capable of predicting the breast cancer survival and recurrence outcomes of an ER+ breast cell sample based on the expression of the identified genes in the sample. Such a model may be generated by any of the algorithms described herein or otherwise known in the art as well as those recognized as equivalent in the art using gene(s) (and subsets thereof) disclosed herein for the identification of breast cancer outcomes. The model provides a means for comparing expression profiles of gene(s) of the subset from the sample against the profiles of reference data used to build the model. The model can compare the sample profile against each of the reference profiles or against a model defining delineations made based upon the reference profiles. Additionally, relative values from the sample profile may be used in comparison with the model or reference profiles.

In a preferred embodiment of the invention, breast cell samples identified as normal and cancerous from the same subject may be analyzed, optionally by use of a single microarray, for their expression profiles of the genes used to generate the model. This provides an advantageous means of identifying survival and recurrence outcomes based on relative differences from the expression profile of the normal sample. These differences can then be used in comparison to differences between normal and individual cancerous reference data which was also used to generate the model.

### FURTHER EMBODIMENTS OF THE INVENTION

Further to the disclosure of possible patient samples provided herein, additional samples for use in the practice of the invention include cell containing samples from a subtype of breast cancer, or samples suspected of being a subtype of cancer. These include samples containing atypical, pre-malignant, or non-breast cells as well as samples suspected of containing any one or more of atypical, pre-malignant, or non-breast cells. Other samples include those from a pre-cancerous, or supposedly pre-cancerous, biopsy as well as from a cancer diagnosed biopsy. Additional samples include those from any bodily mass, including a tumor mass as a non-limiting example.

The samples may be of a frozen or fixed type. Various types of fixed samples may be used, including those prepared from fixation methods, including precipitation methods (using methanol, ethanol, or other alcohols), cross-linking methods (using formaldehyde or gluteraldehyde), as well as other commercial methods, such as RNAlater.

Possible samples include blood, serum, and plasma as well as any sample enriched for epithelial cells, endothelial cells, and/or circulating tumor cell(s). Urine and/or bladder washes may also be used in the practice of the invention, along with a cell pellet or spread, cervical scraps (e.g. PAP smears), endometrial scraps, stool, buccal cells, a cellular aspirate in general (including those from any bodily mass, including a tumor mass), and an exfoliation of cells. Of course tissue samples, including fine-needle aspirates, needle biopsies, and excisional biopsies of tissues may be used in the practice of the invention. Another example is a commercial preparation of cells, such as ThinPrep (from Cytyc).

Measurement of gene expression may be made by any means, including gene amplification, gene deletion, gene methylation, mRNA levels, protein levels, protein-protein interaction, and covalent modification as appropriate. Non-limiting examples include the use of immunohistochemistry (IHC) or immunocytochemistry (ICC)as well as mass spectroscopy. The measurements of course may be used to discriminate (or classify) among two or more populations of subjects or patients based upon the samples used. Preferred methods include the use of any measurement using hybridization of a nucleic acid sequence that is complementary to all or part of HOXB13 or other disclosed sequence in a manner sufficient to determine its expression.

The measurement of expression of sequences, such as that of HOXB13 or a ratio of HOXB13 expression to the expression of another gene, as disclosed herein may be used as a prognostic factor for breast cancer, including for the likelihood or possibility of local recurrence, regional recurrence, contralateral recurrence, distant recurrence, secondary primary, death or survival, relapse free survival, disease free survival, and overall survival. The measurement of expression may also be used to predict the treatment outcome for hormonal treatment (including but not limited to SERMs, SERDs, aromatase inhibitors and irreversible inhibitors of estrogen receptor as described herein), and agents that inhibit or affect the EGF signal transduction pathway, including agents that direct interact with the EGF receptor family (erbitux) and the tyrosine kinase inhibitors (e.g. irressa and tarceva) as non-limiting examples.

The measurement may be part of pre-operative treatment (e.g., neoadjuvant treatment) to predict an outcome, such as complete, intermediate or no response (based on “clinical response” or “pathological response”) as non-limiting examples. The measurement may also be used to predict disease regression, stable disease, or disease progression.

The measurement may be part of post-operative treatment (i.e., adjuvant treatment) to predict local recurrence, regional recurrence, contralateral recurrence, distant recurrence, secondary primary, death or survival, relapse free survival, disease free survival, and overall survival.

The measurement may optionally be made by use of a direct testing device by the patient. Such a device may be available without prescription, such as that provided over the counter.

Uses in Medical Care

The present invention also provides for the measurement of the sequences disclosed herein as part of providing medical care to a patient, including the providing of diagnostic services in support of providing medical care. Thus the invention includes a method in the medical care of a patient, the method comprising measuring the expression of HOXB13, IL17BR, CHDH, and/or QPRT in a cell containing sample obtained from the patient. Alternatively, this method in the medical care of a patient method may be any method as disclosed herein. The sample is optionally a breast cancer sample, and the method optionally includes interpretation of the results as provided herein. The measurement may be for use in relation to any aspect or embodiment of the invention as described herein. The measurement may be preceded by a determination of a need for the measurement, such as that determined by a medical doctor, nurse or other health care provider, or those working under their instruction, or personnel of a health insurance or maintenance organization in approving the performance of the measurement as a basis to request reimbursement or payment for the performance.

In another embodiment, the invention provides for a method of ordering, or receiving an order for, the performance of the above method in the medical care of a patient or other methods described herein. The ordering may be made by a medical doctor, a nurse, or other health care provider, or those working under their instruction, while the receiving, direct or indirect, may be made by any personnel who performs the methods.

The invention also provides methods in the processing of reimbursement or payment for a test, such as the above method in the medical care of a patient or other methods described herein. A method in the processing of reimbursement or payment may comprise indicating that 1) payment has been received, or 2) payment will be made by another payer, or 3) payment remains unpaid on paper or in a database after performance of the method of claim 1. The database may be in any form, with electronic forms such as a computer implemented database included within the scope of the invention. The indicating may be in the form of a code on paper or in the database. The another payer may be any person or entity beyond that to whom a previous request for reimbursement or payment was made.

Alternative, the method may comprise receiving reimbursement or payment for the technical performance of the above method in the medical care of a patient or for the interpretation of the results from said method. Of course the invention also includes embodiments comprising instructing another person or party to receive the reimbursement or payment. The receipt may be from any entity, including an insurance company, health maintenance organization, governmental health agency, or a patient as non-limiting examples. The payment may be in whole or in part. In the case of a patient, the payment may be in the form of a partial payment known as a co-pay.

In yet another embodiment, the method may comprise forwarding or having forwarded a reimbursement or payment request to an insurance company, health maintenance organization, governmental health agency, or to a patient for the performance of the above method in the medical care of a patient or other methods described herein. The request may be made by mail, electronically, telephonically, in person, or by facsimile.

In a further embodiment, the method may comprise receiving indication of approval for payment, or denial of payment, for performance of the above method in the medical care of a patient. Such an indication may come from any person or party to whom a request for reimbursement or payment was made. Non-limiting examples include an insurance company, health maintenance organization, or a governmental health agency, like Medicare or Medicaid as non-limiting examples. The indication may have been by mail, electronically, telephonically, in person, or by facsimile.

An additional embodiment is where the method comprises sending a request for reimbursement for performance of the above method in the medical care of a patient or other methods described herein. Such a request may be made by mail, electronically, telephonically, in person, or by facsimile. The request may have been made to an insurance company, health maintenance organization, federal health agency, or the patient for whom the method was performed.

A further method comprises indicating the need for reimbursement or payment on a form or into a database for performance of the above method in the medical care of a patient or other methods described herein. Alternatively, the method may simply indicate the performance of the method. The database may be in any form, with electronic forms such as a computer implemented database included within the scope of the invention. The indicating may be in the form of a code on paper or in the database.

In the above method in the medical care of a patient or other methods described herein, the method may comprise reporting the results of the method, optionally to a health care facility, a health care provider, a doctor, a nurse, or personnel working therefore. The reporting may also be directly or indirectly to the patient. The reporting may have been by mail, electronically, telephonically, in person, or by facsimile.

Articles of Manufacture

The materials and methods of the present invention are ideally suited for preparation of kits produced in accordance with well known procedures. The invention thus provides kits comprising agents (like the polynucleotides and/or antibodies described herein as non-limiting examples) for the detection of expression of the disclosed sequences. Such kits, optionally comprising the agent with an identifying description or label or instructions relating to their use in the methods of the present invention, are provided. Such a kit may comprise containers, each with one or more of the various reagents (typically in concentrated form) utilized in the methods, including, for example, pre-fabricated microarrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more primer complexes of the present invention (e.g., appropriate length poly(T) or random primers linked to a promoter reactive with the RNA polymerase). A set of instructions will also typically be included.

The methods provided by the present invention may also be automated in whole or in part. All aspects of the present invention may also be practiced such that they consist essentially of a subset of the disclosed genes to the exclusion of material irrelevant to the identification of breast cancer survival outcomes via a cell containing sample.

Having now generally described the invention, the same will be more readily understood through reference to the following examples which are provided by way of illustration, and are not intended to be limiting of the present invention, unless specified.

### EXAMPLES

### Example 1

### General Methods

Patient and Tumor Selection Criteria and Study Design

Patient inclusion criteria for this study were: Women diagnosed at the Massachusetts General Hospital (MGH) between 1987 and 2000 with ER positive breast cancer, treatment with standard breast surgery (modified radical mastectomy or lumpectomy) and radiation followed by five years of systemic adjuvant tamoxifen; no patient received chemotherapy prior to recurrence. Clinical and follow-up data were derived from the MGH tumor registry. There were no missing registry data and all available medical records were reviewed as a second tier of data confirmation.

All tumor specimens collected at the time of initial diagnosis were obtained from frozen and formalin fixed paraffin-embedded (FFPE) tissue repositories at the Massachusetts General Hospital. Tumor samples with greater than 20% tumor cells were selected with a median of greater than 75% for all samples. Each sample was evaluated for the following features: tumor type (ductal vs. lobular), tumor size, and Nottingham combined histological grade. Estrogen and progesterone receptor expression were determined by biochemical hormone binding analysis and/or by immunohistochemical staining as described (Long, A. A. et al. “High-specificity in-situ hybridization. Methods and application.” Diagn Mol Pathol 1, 45-57 (1992)); receptor positivity was defined as greater than 3 fmol/mg tumor tissue (Long et al.) and greater than 1% nuclear staining for the biochemical and immunohistochemical assays, respectively.

Study design is as follows: A training set of 60 frozen breast cancer specimens was selected to identify gene expression signatures predictive of outcome or response, in the setting of adjuvant tamoxifen therapy. Tumors from responders were matched to the non-responders with respect to TNM staging and tumor grade. Differential gene expression identified in the training set was validated in an independent group of 20 invasive breast tumors with formalin fixed paraffin-embedded (FFPE) tissue samples.

LCM, RNA Isolation and Amplification

With each frozen tumor sample within the 60-case cohort, RNA was isolated from both a whole tissue section of 8 μm in thickness and a highly enriched population of 4,000-5,000 malignant epithelial cells acquired by laser capture microdissection using a PixCell IIe LCM system (Arcturus, Mountain View, Calif.). From each tumor sample within the 20-case test set, RNA was isolated from four 8 μm-thick FFPE tissue sections. Isolated RNA was subjected to one round of T7 polymerase in vitro transcription using the RiboAmp™ kit (frozen samples) or another system for FFPE samples according to manufacturer's instructions (Arcturus Bioscience, Inc., Mountain View, Calif. for RiboAmp™). Labeled cRNA was generated by a second round of T7-based RNA in vitro transcription in the presence of 5-[3-Aminoallyl]uridine 5′-triphosphate (Sigma-Aldrich, St. Louis, Mo.). Universal Human Reference RNA (Stratagene, San Diego, Calif.) was amplified in the same manner. The purified aRNA was later conjugated to Cy5 (experimental samples) or Cy3 (reference sample) dye (Amersham Biosciences).

Microarray Analysis

A custom designed 22,000-gene oligonucleotide (60mer) microarray was fabricated using ink-jet in-situ synthesis technology (Agilent Technologies, Palo Alto, Calif.). Cy5-labeled sample RNA and Cy3-labeled reference RNA were co-hybridized at 65° C., 1× hybridization buffer (Agilent Technologies). Slides were washed at 37° C. with 0.1×SSC/0.005% Triton X-102. Image analysis was performed using Agilent's image analysis software. Raw Cy5/Cy3 ratios were normalized using intensity-dependent non-linear regression.

A data matrix consisting of normalized Cy5/Cy3 ratios from all samples were median centered for each gene. The variance of each gene over all samples was calculated and the top 25% high variance genes (5,475) selected for further analysis. Identification and permutation testing for significance of differential gene expression were performed using BRB ArrayTools, developed by Dr. Richard Simon and Amy Peng (see http site at linus.nci.nih.gov/BRB-ArrayTools.html). Hierarchical cluster analysis was performed with GeneMaths software (Applied-Maths, Belgium) using cosine correlation and complete linkage. All other statistical procedures (two-sample t-test, receiver operating characteristic analysis, multivariate logistic regression and survival analysis) were performed in the open source R statistical environment (see http site at www.r-project.org). Statistical test of significance of ROC curves was by the method of DeLong (“Comparing the areas under two or more correlated receiver operating characteristic curves: a nonparametric approach.” Biometrics 44, 83745 (1988)). Disease free survival was calculated from the date of diagnosis. Events were scored as the first distant metastasis, and patients remaining disease-free at the last follow-up were censored. Survival curves were calculated by the Kaplan-Meier estimates and compared by log-rank tests.

Real-Time Quantitative PCR Analysis

Real-time PCR was performed on 59 of the 60-case training samples (one case was excluded due to insufficient materials) and the 20-case validation samples. Briefly, 2 μg of amplified RNA was converted into double stranded cDNA. For each case 12ng of cDNA in triplicates was used for real-time PCR with an ABI 7900HT (Applied Biosystems) as described (Gelmini, S. et al. “Quantitative polymerase chain reaction-based homogeneous assay with fluorogenic probes to measure c-erbB-2 oncogene amplification.” Clin Chem 43, 752-8 (1997)). The sequences of the PCR primer pairs and fluorogenic MGB probe (5′ to 3′), respectively, that were used for each gene are as follows:

HoxB13
TTCATCCTGACAGTGGCAATAATC,
CTAGATAGAAAATATGAGGCTAACGATCAT,
VIC-CGATAACCAGTACTAGCTG;
IL17BR
GCATTAACTAACGATTGGAAACTACATT,
GGAAGATGCTTTATTGTTGCATTATC,
VIC-ACAACTTCAAAGCTGTTTTA.

Relative expression levels of HOXB13 in normal, DCIS and IDC samples were calculated as follows. First, all CT values are adjusted by subtracting the highest CT (40) among all samples, then relative expression=1/2ˆACT.

In Situ Hybridization

Dig-labeled RNA probes were prepared using DIG RNA labeling kit (SP6/T7) from Roche Applied Science, following the protocol provided with the kit. In situ hybridization was performed on frozen tissue sections as described (Long et al.).

TABLE 1
Patients and tumor characteristics of training set.

| Sample | | | | | | | | | |
|---|---|---|---|---|---|---|---|---|---|
| ID | Tumor type | Size | Grade | Nodes | ER | PR | Age | DFS | Status |
| 1389 | D | 1.7 | 2 | 0/1 | Pos | Pos | 80 | 94 | 0 |
| 648 | D | 1.1 | 2 | 0/15 | Pos | ND | 62 | 160 | 0 |
| 289 | D | 3 | 2 | 0/15 | Pos | ND | 75 | 63 | 1 |
| 749 | D | 1.8 | 2 | 2/9 | Pos | Pos | 61 | 137 | 0 |
| 420 | D/L | 2 | 3 | ND | Pos | Pos | 72 | 58 | 1 |
| 633 | D | 2.7 | 3 | 0/11 | Pos | ND | 61 | 20 | 1 |
| 662 | D | 1 | 3 | 6/11 | Pos | Pos | 79 | 27 | 1 |
| 849 | D | 2 | 1 | 0/26 | Pos | Neg | 75 | 23 | 1 |
| 356 | D | 1 | 2 | 2/20 | Pos | ND | 58 | 24 | 1 |
| 1304 | D | 2 | 3 | 0/14 | Pos | Pos | 57 | 20 | 1 |
| 1419 | D | 2.5 | 2 | 1/8 | Pos | Pos | 59 | 86 | 0 |
| 1093 | D | 1 | 3 | 1/14 | Pos | Pos | 66 | 85 | 0 |
| 1047 | D/L | 2.6 | 2 | 0/18 | Pos | Neg | 70 | 128 | 0 |
| 1037 | D/L | 1.5 | 2 | 0/4 | Pos | Pos | 85 | 83 | 0 |
| 319 | D | 4 | 2 | 1/13 | Pos | ND | 67 | 44 | 1 |
| 25 | D | 3.5 | 2 | 0/9 | Neg | Pos | 62 | 75 | 1 |
| 180 | D | 1.6 | 2 | 2/19 | Pos | Pos | 69 | 169 | 0 |
| 687 | D | 3.5 | 3 | 3/16 | Pos | ND | 73 | 142 | 0 |
| 856 | D | 1.6 | 2 | 0/16 | Pos | Pos | 73 | 88 | 0 |
| 1045 | D | 2.5 | 3 | 1/12 | Pos | Neg | 73 | 121 | 0 |
| 1205 | D | 2.7 | 2 | 1/19 | Pos | Pos | 71 | 88 | 0 |
| 1437 | D | 1.7 | 2 | 2/22 | Pos | Pos | 67 | 89 | 0 |
| 1507 | D | 3.7 | 3 | 0/40 | Pos | Pos | 70 | 70 | 0 |
| 469 | D | 1 | 1 | 0/19 | Pos | ND | 66 | 161 | 0 |
| 829 | D | 1.2 | 2 | 0/9 | Pos | ND | 69 | 136 | 0 |
| 868 | D | 3 | 3 | 0/13 | Pos | Pos | 65 | 130 | 0 |
| 1206 | D | 4.1 | 3 | 0/15 | Pos | Neg | 84 | 56 | 1 |
| 843 | D | 3.4 | 2 | 11/20 | Pos | Neg | 76 | 122 | 1 |
| 342 | D | 3 | 2 | 9/21 | Pos | ND | 62 | 102 | 1 |
| 1218 | D | 4.5 | 1 | 3/16 | Pos | Pos | 62 | 10 | 1 |
| 547 | D/L | 1.5 | 2 | ND | Pos | ND | 74 | 129 | 1 |
| 1125 | D | 2.6 | 2 | 0/18 | Pos | Pos | 54 | 123 | 0 |
| 1368 | D | 2.6 | 2 | ND | Pos | Pos | 82 | 63 | 0 |
| 605 | D | 2.2 | 2 | 6/18 | Pos | ND | 70 | 110 | 0 |
| 59 | L | 3 | 2 | 33/38 | Pos | ND | 70 | 21 | 1 |
| 68 | D | 3 | 2 | 0/17 | Pos | ND | 53 | 38 | 1 |
| 317 | D | 1.2 | 3 | 1/10 | Pos | Pos | 71 | 5 | 1 |
| 374 | D | 1 | 3 | 0/15 | Pos | Neg | 57 | 47 | 1 |
| 823 | D | 2 | 2 | 0/6 | Pos | Pos | 51 | 69 | 1 |
| 280 | D | 2.2 | 3 | 0/12 | Pos | ND | 66 | 44 | 1 |
| 651 | D | 4.7 | 3 | 10/13 | Pos | ND | 48 | 137 | 1 |
| 763 | D | 1.8 | 2 | 0/14 | Pos | Pos | 63 | 118 | 0 |
| 1085 | D | 4.7 | 2 | 0/8 | Pos | Pos | 48 | 101 | 1 |
| 1363 | D | 2.1 | 2 | 0/15 | Pos | Pos | 56 | 114 | 0 |
| 295 | D | 3.5 | 2 | 3/21 | Pos | Pos | 52 | 118 | 1 |
| 871 | D | 4 | 3 | 0/16 | Pos | Neg | 61 | 6 | 1 |
| 1343 | D | 2.5 | 3 | ND | Pos | Pos | 79 | 21 | 1 |
| 140 | L | >2.0 | 2 | 18/28 | Pos | ND | 63 | 43 | 1 |
| 260 | D/L | 0.9 | 2 | 1/13 | Pos | ND | 73 | 42 | 1 |
| 297 | D | 0.8 | 2 | 1/16 | Pos | Pos | 66 | 169 | 0 |
| 1260 | D | 3.5 | 2 | 0/14 | Pos | Pos | 58 | 79 | 0 |
| 1405 | D | 1 | 3 | ND | Pos | Pos | 81 | 95 | 0 |
| 518 | L | 5.5 | 2 | 3/20 | Pos | ND | 68 | 156 | 0 |
| 607 | D | 1.2 | 2 | 5/14 | Pos | Pos | 76 | 114 | 0 |
| 638 | D | 2 | 2 | 1/24 | Pos | Pos | 67 | 148 | 0 |
| 655 | D | 2 | 3 | ND | Pos | Pos | 73 | 143 | 0 |
| 772 | D | 2.5 | 2 | 0/18 | Pos | Pos | 68 | 69 | 1 |
| 878 | D/L | 1.6 | 2 | 0/9 | Pos | Neg | 76 | 138 | 0 |
| 1279 | D | 2 | 2 | 0/12 | Pos | Pos | 68 | 102 | 0 |
| 1370 | D | 2 | 2 | ND | Pos | Pos | 73 | 61 | 0 |

Abbreviations:
D, ductal;
L, lobular;
D/L. ductal and lobular features;
pos, positive;
neg, negative;
ND, not determined;
ER, estrogen receptor;
PR, progesterone receptor;
DFS, disease-free survival (number of months);
status = 1, recurred;
status = 0, disease-free.

### Example 2

### Identification of Differentially Expressed Genes

Gene expression profiling was performed using a 22,000-gene oligonucleotide microarray as described above. In the initial analysis, isolated RNA from frozen tumor-tissue sections taken from the archived primary biopsies were used. The resulting expression dataset was first filtered based on overall variance of each gene with the top 5,475 high-variance genes (75th percentile) selected for further analysis. Using this reduced dataset, t-test was performed on each gene comparing the tamoxifen responders and non-responders, leading to identification of 19 differentially expressed genes at the P value cutoff of 0.001 (Table 2). The probability of selecting this many or more differentially expressed genes by chance was estimated to be 0.04 by randomly permuting the patient class with respect to treatment outcome and repeating the t-test procedure 2,000 times. This analysis thus demonstrated the existence of statistically significant differences in gene expression between the primary breast cancers of tamoxifen responders and non-responders.

TABLE 2
19-gene signature identified by t-test in the Sections dataset

| | | | | Fold | | |
|---|---|---|---|---|---|---|
| | | | Mean in | difference | | |
| | Parametric | Mean in | non- | of | | |
| | p-value | responders | responders | means | GB acc | Description |
| 1 | 1.96E−05 | 0.759 | 1.317 | 0.576 | AW006861 | SCYA4\|small inducible cytokine |
| | | | | | | A4 |
| 2 | 2.43E−05 | 1.31 | 0.704 | 1.861 | AI240933 | ESTs |
| 3 | 8.08E−05 | 0.768 | 1.424 | 0.539 | X59770 | IL1R2\|interleukin 1 receptor, |
| | | | | | | type II |
| 4 | 9.57E−05 | 0.883 | 1.425 | 0.62 | AB000520 | APS\|adaptor protein with |
| | | | | | | pleckstrin homology and src |
| | | | | | | homology 2 domains |
| 5 | 9.91E−05 | 1.704 | 0.659 | 2.586 | AF208111 | IL17BR\|interleukin 17B receptor |
| 6 | 0.0001833 | 0.831 | 1.33 | 0.625 | AI820604 | ESTs |
| 7 | 0.0001935 | 0.853 | 1.459 | 0.585 | AI087057 | DOK2\|docking protein 2, 56 kD |
| 8 | 0.0001959 | 1.29 | 0.641 | 2.012 | AJ272267 | CHDH\|choline dehydrogenase |
| 9 | 0.0002218 | 1.801 | 0.943 | 1.91 | N30081 | ESTs, Weakly similar to I38022 |
| | | | | | | hypothetical protein [H. sapiens] |
| 10 | 0.0004234 | 1.055 | 2.443 | 0.432 | AI700363 | ESTs |
| 11 | 0.0004357 | 0.451 | 1.57 | 0.287 | AL117406 | ABCC11\|ATP-binding cassette, |
| | | | | | | sub-family C (CFTR/MRP), |
| | | | | | | member 11 |
| 12 | 0.0004372 | 1.12 | 3.702 | 0.303 | BC007092 | HOXB13\|homeo box B13 |
| 13 | 0.0005436 | 0.754 | 1.613 | 0.467 | M92432 | GUCY2D\|guanylate cyclase 2D, |
| | | | | | | membrane (retina-specific) |
| 14 | 0.0005859 | 1.315 | 0.578 | 2.275 | AL050227 | Homo sapiens mRNA; cDNA |
| | | | | | | DKFZp586M0723 (from clone |
| | | | | | | DKFZp586M0723) |
| 15 | 0.000635 | 1.382 | 0.576 | 2.399 | AW613732 | Homo sapiens cDNA FLJ31137 |
| | | | | | | fis, clone IMR322001049 |
| 16 | 0.0008714 | 0.794 | 1.252 | 0.634 | BC007783 | SCYA3\|small inducible cytokine |
| | | | | | | A3 |
| 17 | 0.0008912 | 2.572 | 1.033 | 2.49 | X81896 | C11orf25\|chromosome 11 open |
| | | | | | | reading frame 25 |
| 18 | 0.0009108 | 0.939 | 1.913 | 0.491 | BC004960 | MGC10955\|hypothetical protein |
| | | | | | | MGC10955 |
| 19 | 0.0009924 | 1.145 | 0.719 | 1.592 | AK027250 | Homo sapiens cDNA: FLJ23597 |
| | | | | | | fis, clone LNG15281 |

To refine our analysis to the tumor cells and circumvent potential variability attributable to stromal cell contamination, the same cohort was reanalyzed following laser-capture microdissection (LCM) of tumor cells within each tissue section. Using variance based gene filtering and t-test screening identical to that utilized for the whole tissue section dataset, 9 differentially expressed gene sequences were identified with P<0.001 (Table 3).

TABLE 3
9-gene signature identified by t-test in the LCM dataset

| | | | Mean in | Fold | | |
|---|---|---|---|---|---|---|
| | Parametric | Mean in | non- | difference | | |
| | p-value | responders | responders | of means | GB acc | Description |
| 1 | 2.67E−05 | 1.101 | 4.891 | 0.225 | BC007092 | HOXB13\|homeo box B13 |
| 2 | 0.0003393 | 1.045 | 2.607 | 0.401 | AI700363 | ESTs |
| 3 | 0.0003736 | 0.64 | 1.414 | 0.453 | NM_014298 | QPRT\|quinolinate |
| | | | | | | phosphoribosyltransferase |
| | | | | | | (nicotinate-nucleotide |
| | | | | | | pyrophosphorylase |
| | | | | | | (carboxylating)) |
| 4 | 0.0003777 | 1.642 | 0.694 | 2.366 | AF208111 | IL17BR\|interleukin 17B |
| | | | | | | receptor |
| 5 | 0.0003895 | 0.631 | 1.651 | 0.382 | AF033199 | ZNF204\|zinc finger protein |
| | | | | | | 204 |
| 6 | 0.0004524 | 1.97 | 0.576 | 3.42 | AI688494 | FLJ13189\|hypothetical |
| | | | | | | protein FLJ13189 |
| 7 | 0.0005329 | 1.178 | 0.694 | 1.697 | AI240933 | ESTs |
| 8 | 0.0007403 | 0.99 | 1.671 | 0.592 | AL157459 | Homo sapiens mRNA; cDNA |
| | | | | | | DKFZp434B0425 (from clone |
| | | | | | | DKFZp434B0425) |
| 9 | 0.0007739 | 0.723 | 1.228 | 0.589 | BC002480 | FLJ13352\|hypothetical |
| | | | | | | protein FLJ13352 |

Only 3 genes were identified as differentially expressed in both the LCM and whole tissue section analyses: the homeobox gene HOXB13 (identified twice as AI700363 and BC007092), the interleukin 17B receptor IL17BR (AF208111), and the voltage-gated calcium channel CACNA1D (AI240933). HOXB13 was differentially overexpressed in tamoxifen nonresponsive cases, whereas IL17BR and CACNA1D were overexpressed in tamoxifen responsive cases. Interestingly, the QPRT sequence had similarities to the HOXB13 sequence in relation to expression levels in responders and non-responders. Based on their identification as tumor-derived markers significantly associated with clinical outcome in two independent analyses, the utility of each of these genes was evaluated by itself and in combination with the others.

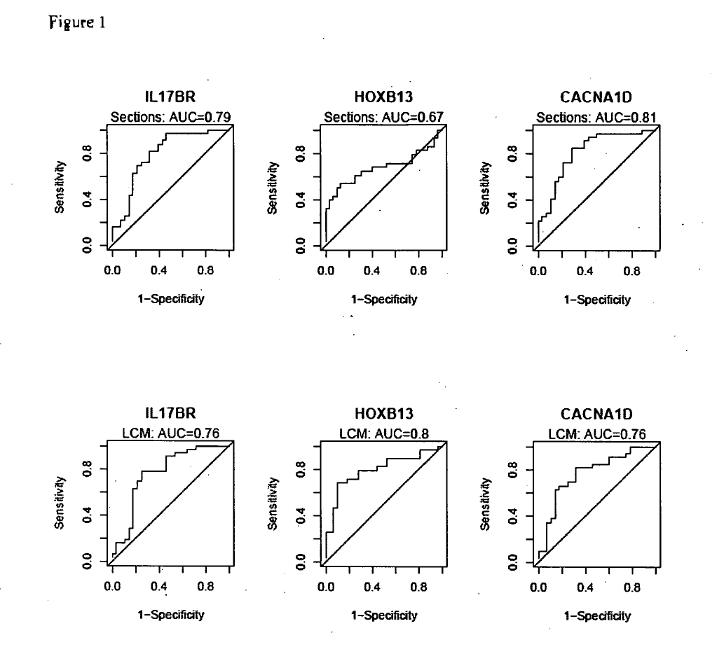

To define the sensitivity and specificity of HOXB13, IL17BR and CACNA1D expression as markers of clinical outcome, Receiver Operating Characteristic (ROC) analysis (Pepe, M. S. “An interpretation for the ROC curve and inference using GLM procedures.” Biometrics 56, 352-9 (2000)) was used. For data derived from whole tissue sections, the Area Under the Curve (AUC) values for IL17BR, HOXB13 and CACNA1D were 0.79, 0.67 and 0.81 for IL17BR, HOXB13 and CACNA1D, respectively (see Table 4 and FIG. 1, upper portion). ROC analysis of the data generated from the microdissected tumor cells yielded AUC values of 0.76, 0.8, and 0.76 for these genes (see Table 4 and FIG. 1, lower portion).

TABLE 4
ROC analysis of using IL17BR, CACNA1D and HOXB13
expression to predict tamoxifen response

| | |
|---|---|
| Tissue Sections | LCM |

| | AUC | P value | AUC | P value |
|---|---|---|---|---|
| IL17BR | 0.79 | 1.58E−06 | 0.76 | 2.73E−05 |
| CACNA1D | 0.81 | 3.02E−08 | 0.76 | 1.59E−05 |
| HOXB13 | 0.67 | 0.012 | 0.79 | 9.94E−07 |
| ESR1 | 0.55 | 0.277 | 0.63 | 0.038 |
| PGR | 0.63 | 0.036 | 0.63 | 0.033 |
| ERBB2 | 0.69 | 0.004 | 0.64 | 0.027 |
| EGFR | 0.56 | 0.200 | 0.61 | 0.068 |

AUC, area under the curve;
P values are AUC > 0.5.

A statistical test of significance indicated that these AUC values are all significantly greater than 0.5, the expected value from the null model that predicts clinical outcome randomly. Therefore, these three genes have potential utility for predicting clinical outcome of adjuvant tamoxifen therapy. As comparison, markers that are currently useful in evaluating the likelihood of response to tamoxifen were analyzed in comparison. The levels of ER (gene symbol ESR1) and progesterone receptor (PR, gene symbol PGR) are known to be positively correlated with tamoxifen response (see Fernandez, M. D., et al. “Quantitative oestrogen and progesterone receptor values in primary breast cancer and predictability of response to endocrine therapy.” Clin Oncol 9, 245-50 (1983); Ferno, M. et al. “Results of two or five years of adjuvant tamoxifen correlated to steroid receptor and S-phase levels.” South Sweden Breast Cancer Group, and South-East Sweden Breast Cancer Group. Breast Cancer Res Treat 59, 69-76 (2000); Nardelli, G. B., et al. “Estrogen and progesterone receptors status in the prediction of response of breast cancer to endocrine therapy (preliminary report).” Eur J Gynaecol Oncol 7, 151-8 (1986); and Osborne, C. K., et al. “The value of estrogen and progesterone receptors in the treatment of breast cancer.” U 46, 2884-8 (1980)).

In addition, growth factor signaling pathways (EGFR, ERBB2) are thought to negatively regulate estrogen-dependent signaling, and hence contribute to loss of responsiveness to tamoxifen (see Dowsett, M. “Overexpression of HER-2 as a resistance mechanism to hormonal therapy for breast cancer.” Endocr Relat Cancer 8, 191-5 (2001)). ROC analysis of these genes confirmed their correlation with clinical outcome, but with AUC values ranging only from 0.55 to 0.69, reaching statistical significance for PGR and ERBB2 (see Table 4).

The LCM dataset is particularly relevant, since EGFR, ERBB2, ESR1 and PGR are currently measured at the tumor cell level using either immunohistochemistry or fluorescence in situ hybridization. As individual markers of clinical outcome, HOXB13, IL17BR and CAC1D all outperformed ESR1, PGR, EGFR and ERBB2 (see Table 4).

### Example 3

### Identification of the HOXB13:IL17BR Expression Ratio

HOXB13:IL17BR expression ratio was identified as a robust composite predictor of outcome as follows. Since HOXB13 and IL17BR have opposing patterns of expression, the expression ratio of HOXB13 over IL17BR was examined to determine whether it provides a better composite predictor of tamoxifen response. Indeed, both t-test and ROC analyses demonstrated that the two-gene ratio had a stronger correlation with treatment outcome than either gene alone, both in the whole tissue sections and LCM datasets (see Table 5). AUC values for HOXB13:IL17BR reached 0.81 for the tissue sections dataset and 0.84 for the LCM dataset. Pairing HOXB13 with CACNA1D or analysis of all three markers together did not provide additional predictive power.

TABLE 5
HOXB13:IL17BR ratio is a stronger predictor of treatment outcome

| | | t-test | | ROC | |
|---|---|---|---|---|---|
| | | t-statistic | P value | AUC | P value |
| Tissue | IL17BR | 4.15 | 1.15E−04 | 0.79 | 1.58E−06 |
| Section | HOXB13 | −3.57 | 1.03E−03 | 0.67 | 0.01 |
| | HOXB13:IL17BR | −4.91 | 1.48E−05 | 0.81 | 1.08E−07 |
| LCM | IL17BR | 3.70 | 5.44E−04 | 0.76 | 2.73E−05 |
| | HOXB13 | −4.39 | 8.00E−05 | 0.79 | 9.94E−07 |
| | HOXB13:IL17BR | −5.42 | 2.47E−06 | 0.84 | 4.40E−11 |

AUC, area under the curve;
P values are AUC > 0.5.

The HOXB13/IL7BR ratio was compared to well-established prognostic factors for breast cancer, such as patient age, tumor size, grade and lymph node status (see Fitzgibbons, P. L. et al. “Prognostic factors in breast cancer. College of American Pathologists Consensus Statement 1999.” Arch Pathol Lab Med 124, 966-78 (2000)). Univariate logistic regression analysis indicated that only tumor size was marginally significant in this cohort (P=0.04); this was not surprising given that the responder group was closely matched to the non-responder group with respect to tumor size, tumor grade and lymph node status during patient selection. Among the known positive (ESR1 and PGR) and negative (ERBB2 and EGFR) predictors of tamoxifen response, ROC analysis of the tissue sections data indicated that only PGR and ERBB2 were significant (see Table 4). Therefore, a comparison of logistic regression models containing the HOXB13:IL17BR ratio either by itself or in combination with tumor size, and expression levels of PGR and ERBB2, were made (see Table 6). The HOXB13:IL17BR ratio alone was a highly significant predictor (P=0.0003) and had an odds ratio of 10.2 (95% CI 2.9-35.6). In the multivariate model, HOXB13:IL17BR ratio is the only significant variable (P=0.002) with an odds ratio of 7.3 (95% CI 2.1-26). Thus, the expression ratio of HOXB13:IL17BR is a strong independent predictor of treatment outcome in the setting of adjuvant tamoxifen therapy.

TABLE 6
Logistic Regression Analysis

| | | | |
|---|---|---|---|
| | Odds | | |
| Predictor | Ratio | 95% CI | P Value |

Univariate Model

| | | | |
|---|---|---|---|
| HOXB13:IL17BR | 10.17 | 2.9-35.6 | 0.0003 |

Multivariate Model

| | | | |
|---|---|---|---|
| Tumor size | 1.5 | 0.7-3.5 | 0.3289 |
| PGR | 0.8 | 0.3-1.8 | 0.5600 |
| ERBB2 | 1.7 | 0.8-3.8 | 0.1620 |
| HOXB13:IL17BR | 7.3 | 2.1-26.3 | 0.0022 |

All predictors are continuous variables. Gene expression values were from microarray measurements.
Odds ratio is the inter-quartile odds ratio, based on the difference of a predictor from its lower quartile (0.25) to its upper quartile (0.75);
CI, confidence interval.

### Example 4

### Independent Validation of HOXB13: IL17BR Expression Ratio

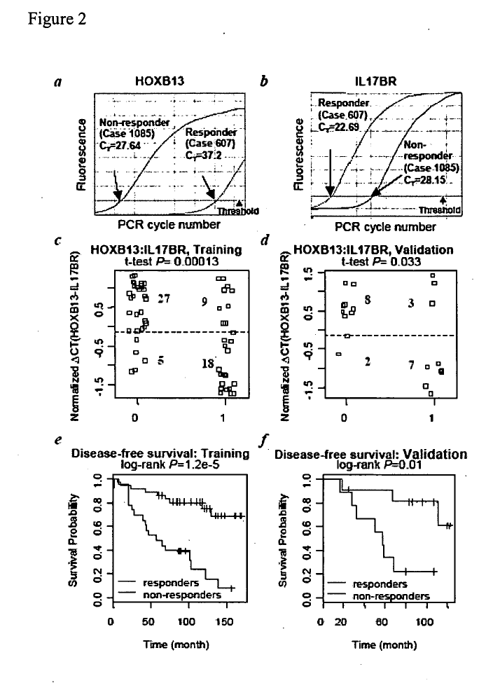

The reduction of a complex microarray signature to a two-gene expression ratio allows the use of simpler detection strategies, such as real-time quantitative PCR (RT-QPCR) analysis. The HOXB13:IL17BR expression ratio by RT-QPCR using frozen tissue sections that were available from 59 of the 60 training cases were analyzed (FIG. 2, part a). RT-QPCR data were highly concordant with the microarray data of frozen tumor specimens (correlation coefficient r=0.83 for HOXB13, 0.93 for IL17BR). In addition, the PCR-derived HOXB13:IL17BR ratios, represented as ACTs, where CT is the PCR amplification cycles to reach a predetermined threshold amount (e.g., FIG. 2, parts a and b) and ΔCT is the CT difference between HOXB13 and IL17BR, were highly correlated with the microarray-derived data (r=0.83) and with treatment outcome (t test P=0.0001, FIG. 2, part c). Thus, conventional RT-QPCR analysis for the expression ratio of HOXB13 to IL17BR appears to be equivalent to microarray-based analysis of frozen tumor specimens.

To validate the predictive utility of HOXB13:IL17BR expression ratio in an independent patient cohort, 20 additional ER-positive early-stage primary breast tumors from women treated with adjuvant tamoxifen only at MGH between 1991 and 2000, and for which medical records and paraffin-embedded tissues were available, were identified. Of the 20 archival cases, 10 had recurred with a median time to recurrence of 5 years, and 10 had remained disease-free with a median follow up of 9 years (see Table 7 for details).

TABLE 7
Patient and tumor characteristics of the validation set.

| Sample | Tumor Type | Size | Grade | Nodes | ER | PR | Age | DFS | Status |
|---|---|---|---|---|---|---|---|---|---|
| Test 1 | D | 1.9 | 3 | 0/10 | Pos | Pos | 69 | 15 | 1 |
| Test 2 | D | 1.7 | 3 | 0/19 | Pos | Pos | 61 | 117 | 1 |
| Test 3 | D | 1.7 | 2 | 0/26 | Pos | ND | 65 | 18 | 1 |
| Test 4 | D | 1.2 | 2 | 0/19 | Pos | Pos | 63 | 69 | 1 |
| Test 5 | D | 1.7 | 2 | 2/2 | Pos | Pos | 60 | 5 | 1 |
| Test 6 | D | 1.1 | 1 | 0/10 | Pos | Pos | 54 | 59 | 1 |
| Test 7 | D | >1.6 | 2 | 0/17 | Pos | Neg | 66 | 32 | 1 |
| Test 8 | L | 2.6 | 1-2 | 0/14 | Pos | Pos | 58 | 67 | 1 |
| Test 9 | D | 1.2 | 2 | ND | Pos | Pos | 93 | 58 | 1 |
| Test 10 | D | 4 | 3 | 0/20 | Pos | Pos | 66 | 27 | 1 |
| Test11 | D | 1.1 | 2 | 0/19 | Pos | Pos | 64 | 97 | 0 |
| Test 12 | D | 2.7 | 2 | 0/10 | Pos | Pos | 66 | 120 | 0 |
| Test 13 | D | 0.9 | 1 | 0/22 | Pos | Pos | 66 | 123 | 0 |
| Test 14 | D | 2.1 | 2 | 0/16 | Pos | Pos | 57 | 83 | 0 |
| Test 15 | D | 0.8 | 1-2 | 0/8 | Pos | Pos | 74 | 80 | 0 |
| Test 16 | D | 1 | 2 | 0/13 | Pos | Pos | 74 | 93 | 0 |
| Test 17 | D | 1.6 | 2 | 0/29 | Pos | Pos | 66 | 121 | 0 |
| Test 18 | L | 1.5 | 1-2 | 0/8 | Pos | Pos | 65 | 25 | 0 |
| Test 19 | D | 1.5 | 3 | 0/16 | Pos | Pos | 60 | 108 | 0 |
| Test 20* | L | 4 | 1-2 | 0/19 | Pos | Pos | 60 | 108 | 0 |

Abbreviations: Same as supplemental Table 1.
*Patient received tamoxifen for 2 years.

RNA was extracted from formalin-fixed paraffin-embedded (FFPE) whole tissue sections, linearly amplified, and used as template for RT-QPCR analysis. Consistent with the results of the training cohort, the HOXB13:IL17BR expression ratio in this independent patient cohort was highly correlated with clinical outcome (t test P=0.035) with higher HOXB13 expression (lower ACTs) correlating with poor outcome (FIG. 2, part d). To test the predictive accuracy of the HOXB13:IL17BR ratio, the RT-QPCR data from the frozen tissue sections (n=59) was used to build a logistic regression model. In this training set, the model predicted treatment outcome with an overall accuracy of 76% (P=0.000065, 95% confidence interval 63%-86%). The positive and negative predictive values were 78% and 75%, respectively. Applying this model to the 20 independent patients in the validation cohort, treatment outcome for 15 of the 20 patients was correctly predicted (overall accuracy 75%, P=0.04, 95% confidence interval 51%-91%), with positive and negative predictive values of 78% and 73%, respectively.

Kaplan-Meier analysis of the patient groups as predicted by the model resulted in significantly different disease-free survival curves in both the training set and the independent test set (FIG. 2, parts e and f).

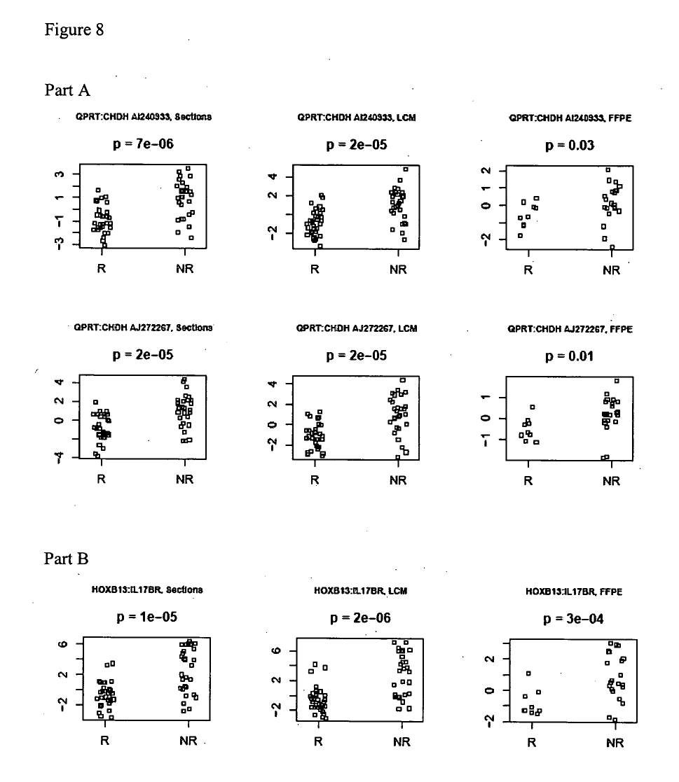

A further representative example of the application of the ratio to section samples and LCM samples of the 60 patient cohort is shown in FIG. 8, Part B (indicated by “Sections” and “LCM” respectively). Also shown therein is an exemplary application of the ratio to 31 FFPE samples (indicated by “FFPE”).

### Example 5

### Identification of Additional Sequences as the Result of CHDH Expression

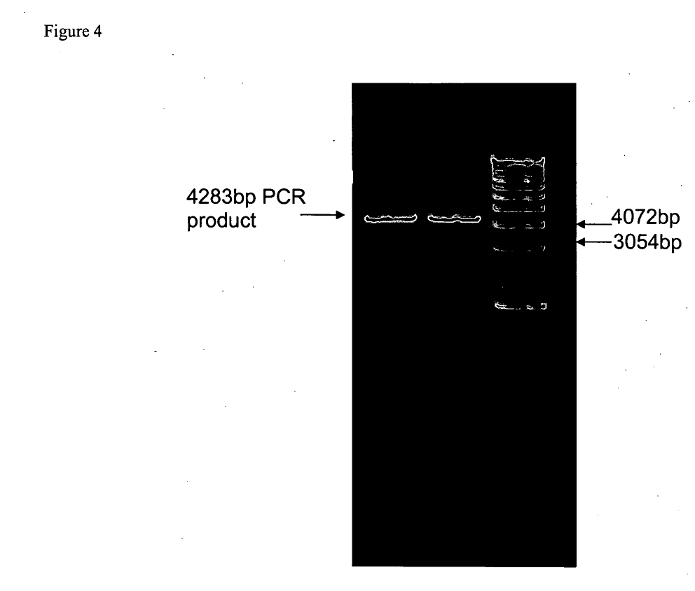

The fact that the sequence of AI240933 was complementary to the coding strand of CACNA1D led to the question of whether expression of a sequence other than that of CACNA1D was detected in Example 2 above. Therefore an assembly of sequences was made, which led to the identification of a larger 3′ region of the known sequences expressed as part of CHDH (see FIGS. 5 and 3). This larger sequence was confirmed to be expressed by PCR analysis using probes that are capable of amplifying a calculated 4283 nucleotide region that spans both portions of previously identified CHDH sequence and the AI240933 sequence (see FIG. 4).

The assembly of sequences in FIG. 5 led to the identification of a larger contig of sequences shown in FIG. 6. Additional support for the likelihood of the larger contig was provided by an alignment of the contig to mouse CHDH sequences.

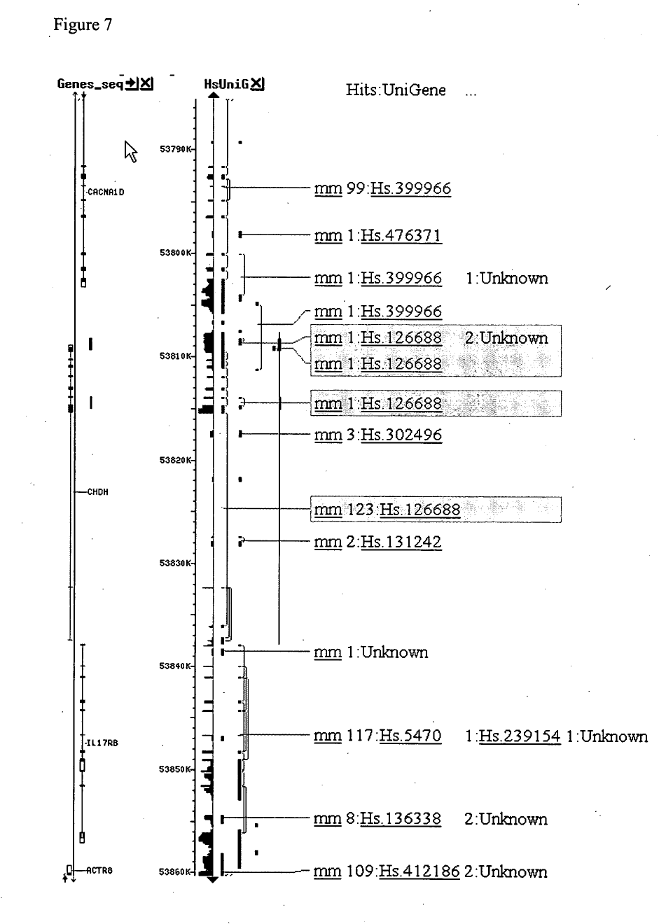

The likely relationship between CHDH and CACNA1D sequences is shown in FIG. 7.

### Example 6

### Identification and Use of the QPRT:CHDH Expression

A QPRT:CHDH expression ratio was identified as a robust composite predictor of outcome in a manner similar to that described in Example 3 above. Since QPRT and CHDH have opposing patterns of expression, the expression ratio of QPRT over CHDH was examined to determine its ability to function as a composite predictor of tamoxifen response. Results from the application of the ratio to section samples and LCM samples of the 60 patient cohort is shown in FIG. 8, Part A (indicated by “Sections” and “LCM” respectively). Also shown therein is an exemplary application of the ratio to 31 FFPE samples (indicated by “FFPE”).

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

We claim:

1. A method in the medical care of a patient, said method comprising measuring the expression of HOXB13, IL17BR, CHDH, and/or QPRT in a cell containing sample of said patient after determination of a need for to measure said expression; and optionally interpreting the results.

2. A method in the processing of reimbursement or payment for a test, said method comprising indicating that 1) payment has been received, or 2) payment will be made by other payer, or 3) payment remains unpaid after performance of the method of claim 1.

3. A method in the processing of reimbursement or payment for a test, said method comprising receiving reimbursement for the technical performance of the method of claim 1 or the interpretation of the results from said method.

4. A method in the processing of reimbursement or payment for a test, said method comprising forwarding or having forwarded a reimbursement request to an insurance company, health maintenance organization, federal health agency, or to said patient for the performance of the method of claim 1.

5. A method in the processing of reimbursement or payment for a test, said method comprising receiving indication of approval for payment or denial of payment for performance of the method of claim 1.

6. A method in the processing of reimbursement or payment for a test, said method comprising sending a request for reimbursement for performance of the method of claim 1.

7. A method in the processing of reimbursement or payment for a test, said method comprising indicating the need for reimbursement or payment on a form or into a database for performance of the method of claim 1.

8. A method in the processing of reimbursement or payment for a test, said method comprising indicating the performance of the method of claim 1 on a form or into a database.

9. A method in the medical care of a patient, said method comprising reporting the results of the method of claim 1, optionally to a health care facility, a health care provider, a doctor, a nurse, or said patient.

10. A method in the medical care of a patient, said method comprising receiving a payment from said patient for the performance of the method of claim 1.
